(19) United States
(12) Patent Application Publication
(10) Pub. No.: US 2006/0276536 A1
(43) Pub. Date: Dec. 7, 2006

(54) Cancer treatment using curcumin derivatives

(76) Inventors: David L. Vander Jagt, Albuquerque, NM (US); Lorraine M. Deck, Albuquerque, NM (US); Steve F. Abcouwer, Hummelstown, PA (US); Ekaterina Bobrovnikova-Marjon, Philadelphia, PA (US); Waylon M. Weber, Albuquerque, NM (US)

Correspondence Address: MUETING, RAASCH & GEBHARDT, P.A., P.O. BOX 581415, MINNEAPOLIS, MN, 55458, US

(21) Appl. No.: 11/373,444

(22) Filed: Mar. 10, 2006

Related U.S. Application Data
(63) Continuation-in-part of application No. 11/057,736, filed on Feb. 14, 2005.
(60) Provisional application No. 60/544,424, filed on Feb. 12, 2004.

(51) Int. Cl. A61K 31/336; A61K 31/192; A61K 31/235; A61K 31/12

(52) U.S. Cl. 514/475; 514/690; 514/569; 514/570; 514/545; 514/679

(57) ABSTRACT

Cancer or a precancerous condition is treated by administering a curcumin derivative to a subject.

### CONTINUING APPLICATION DATA

This application is a continuation-in-part of U.S. patent application Ser. No. 11/057,736, filed Feb. 14, 2005, which claims the benefit of provisional patent application No. 60/544,424, filed Feb. 12, 2004, each of which is incorporated by reference herein.

### GOVERNMENT FUNDING

The present invention was made with government support under Grant No. EY13695, awarded by the National Eye Institute, and Grant No. BC043125, awarded by the U.S. Army/DOD Breast Cancer Program. The Government may have certain rights in this invention.

### BACKGROUND

The transcription factor NF-κB is an established regulator of numerous genes important in the inflammatory response. More recently however, activation of NF-κB has been shown to have a role in many aspects of oncogenesis including control of apoptosis as well as regulation of cell cycling and cell migration (Yamamoto et al., J. Clin. Invest. 2001, 107, 135; Baldwin, A. S. J. Clin. Invest. 2001, 107, 241). Activated NF-κB has been observed in many cancers and is especially important in metastasis (Andela et al., Clin. Orthop. Relat. Res. 2003, 415 (suppl), S75). There are five members to the NF-κB family, distinguished by the presence of an N-terminal Rel homology domain: Rel (c-Rel), RelA (p65), RelB, NF-κB1 (p50 and its precursor p105), and NF-κB2 (p52 and its precursor p100). NF-κB transcription factors are homo- or heterodimers of these members, with the p65/p50 heterodimer being the most common form. NF-κB members are retained in the cytosol as complexes with a set of inhibitory proteins, designated I-κB, where the inhibitory protein masks a nuclear localization signal. In the classical pathway for activation of NF-κB, the upstream I-κB kinase complex (IKK) is first activated in response to many different signals, resulting in phosphorylation of I-κB, followed by its ubiquitination and proteosomal degradation. This is followed by nuclear translocation of NF-κB with resulting activation of a battery of genes, including anti-apoptotic pro-survival genes. There are also alternative pathways for activation of NF-κB (Viatour et al., Trends Biochem. Sci. 2005, 30, 43; Ghosh et al., Cell 2002, 109, S81). A pictorial representation of the NF-κB activation cascade is provided by FIG. 1.

The evidence that links activation of NF-κB to oncogenesis is compelling. NF-κB is activated by a number of viral transforming proteins (Hiscott et al., J. Clin. Invest. 2001, 107, 143), and inhibition of NF-κB activation through expression of a dominant negative IKK can block cell transformation (Arsura et al., Mol. Cell Biol. 2000 20, 5381). NF-κB activation protects cells from apoptosis induced by cancer chemo-therapeutics and oncogenes (Barkett et al., Oncogene 1999, 18, 6910), and activation of NF-κB promotes expression of metastatic factors (Baldwin, A. S. J. Clin. Invest. 2001, 107, 241).

The transcription factor NF-κB, which is well known for its role in inflammatory diseases, is now also known to play an important role in cancer. NF-κB is active in many tumors, and expression of NF-κB-responsive genes provide cancer cells with distinct survival advantages that inhibit cancer treatment. NF-κB is constitutively activated in many cancer cells, and NF-κB may also be conditionally activated in both cancer cells and stromal cells by the tumor microenvironment. Normally, NF-κB activation is prevented by binding to inhibitor (IκB) proteins, the most prevalent being inhibitor of NF-κB alpha (I-κBα). In response to inflammatory cytokines, the release of NF-κB is triggered by phosphorylation of I-κBα on serines 32 and 36, resulting in ubiquination and degradation of I-κBα protein. However, in cancer cells subjected to environmental conditions such as hypoxia, nutrient starvation, or X-rays, NF-κB activation is caused by phosphorylation of I-κBα on a tyrosine residue (Tyr42) by Src family kinases (SFKs). Thus, NF-κB activation via IκBα Tyr42 phosphorylation is expected to occur in solid tumors due to constitutive activation of SFKs such as the Src oncogene in response to the hypoxic and nutrient poor nature of the tumor microenvironment, or due to radiation treatment of the tumor.

Activator Protein-1 (AP-1) is another protein transcription factor found in mammalian cells. AP-1 like NF-κB is a prosurvival and pro-inflammatory protein. AP-1 is an established regulator of numerous genes important in a variety of cellular processes including cell growth regulation, differentiation and proliferation (Angel et al., Cell 1987, 49, 729-739). Growth factors, hormones, tumor promoters and oncogenes regulate AP-1 binding to DNA (Bernstein et al., Science 1989, 244, 566-569). Activated AP-1 has been shown to play a role in apoptosis, angiogenesis and metastasis (Kang et al., Am. J. Pathol. 2005, 166(6), 1691-1699) and is also involved in many diseases including cancer, diabetes and Alzheimer's disease. AP-1 is also associated with the production of metalloproteinases. Collagenases, a class of metalloproteinases, are known to contain AP-1 response elements in their DNA promoters (Kang et al., Am. J. Pathol. 2005, 166(6), 1691-1699). The combination of these factors makes AP-1 crucial to many oncogenic processes.

The AP-1 activation cascade can be induced by TNFα, okadaic acid, 12-O-tetradecanoylphorbol-13-acetate (TPA), UV light (Young et al., Trends Mol. Med. 2003, 9(1), 36-41), cytokines, mitogens, phorbol esters, growth factors, environmental and occupational particles, toxic metals, intracellular stresses, bacterial toxins, viral products and ionizing radiation (Fontecave et al., FEBS Lett. 1998, 421, 277-279). In general, the same factors that stimulate NF-κB also stimulate AP-1.

In normal tissues, the AP-1 component c-Fos is found only in small concentrations but cytostolic levels are rapidly increased when the cell is induced by mitogenic stimuli (Muller et al., Nature 1983, 304, 454-456). c-Jun, another AP-1 component, plays an important role in the regulation of cellular proliferation (Karin et al., Curr. Opin. Cell Biol. 1997, 9(2), 240-246). When c-Jun and c-Fos become unregulated in the body, abnormal cell proliferation occurs leading to cellular transformations. c-Jun is known to be essential in tumor promotion in several cell lines (Jochum et al., Oncogene 2001, 20(19), 2401-2412; Orlowski et al., Trends Mol. Med. 2002, 8, 385-389; Pain, Eur. J. Biochem. 1996, 236, 747-771; Karin et al., Nat. Rev. Cancer 2002, 2(4), 301-310; Dhar et al., Mol. Cell. Biochem. 2002, 234-235, 185-193). c-Fos is also involved in the conversion of cells from benign to malignant (Dong et al., Proc. Natl. Acad. Sci. USA 1994, 91, 609-613; Greenhalgh et al., Cell Growth Differ. 1995, 6, 579-586) and is essential in tumor progression (Saez et al., Cell 1995, 82(5), 721-732). In general, the activation of both NF-κB and AP-1 are required for tumor promotion and progression.

AP-1 consists of 18 dimeric combinations of the families Jun (c-Jun, JunB and JunD) and Fos (c-Fos, FosB, Fra-1 and Fra-2) (Young et al., Trends Mol. Med. 2003, 9(1), 36-41). Of the dimeric possibilities are Jun-Jun homodimers and Jun-Fos heterodimers. Jun dimers bind tightly to AP-1 DNA recognition elements (Angel et al., Cell 1987, 49, 729-739). Fos-Fos homodimers are unstable and not readily formed but can bind to DNA by forming heterodimers with Jun proteins (Ziegler et al., J. Nutr. 2004, 134, 5-10). The most common dimer is a heterodimer consisting of c-Jun and c-Fos. Also associated with the Jun and Fos families are Jun dimerization partners and activating transcription factors (ATF's) (Angel et al., Biochim. Biophys. Acta 1991, 1072, 129-157).

In general, AP-1 is activated primarily through mitogen-activated protein kinase (MAPK) cascades (Kundu et al., Mutat. Res. 2004, 555, 65-80). MAPK's are composed of MAPK itself and MAPK kinase, also called MAPK-extracellular signal regulated kinase (MEK) (Wilkinson et al., Genes Dev. 1998, 12, 1391-1397). MAPK's are activated by cytokines, hormones and stress-inducing agents (Blenis, Proc. Natl. Acad. Sci. USA 1993, 90(13), 5889-5892). MAPK or MEK can phosphorylate additional kinases including extracellular regulating kinases (ERK's), c-Jun N-terminal kinase (JNK) and p38 MAPK (Baker et al., Mol. Cell. Biol. 1992, 12(10), 4694-4705; Davis, J. Biol. Chem. 1993, 268(20), 14553-14556). JNK activates the c-Jun protein and ERK activates a protein called Elk-1 both by phosphorylation. c-Jun then binds to DNA along with an ATF to activate genes that produce more of the Jun family in a positive feedback loop (Thevenin et al., J. Biol. Chem. 1991, 266(15), 9363-9366). Elk-1 also binds to DNA with a serum response factor (SRF) to activate genes that produce the Fos family. The Jun and Fos protein families are then activated by JNK and c-Fos-regulating kinase (FRK) respectively. The activated families can now dimerize, bind to DNA and activate gene expression that adversely affects cellular processes. A pictorial representation of AP-1 activation is shown in FIG. 2.

AP-1 proteins and their activating kinases are related to NF-κB. AP-1 proteins are known to interact with the p65 subunit of NF-κB (Li et al., Mol. Carcinog. 2000, 29(3), 159-169). MAPK's are known to phosphorylate IκB (Adler et al., EMBO J. 1999, 18, 1321-1334). Curcumin is known to inhibit the formation of Jun-Fos heterodimers in TPA induced cells and curcumin analogs are known to be up to 90 times more potent than curcumin (Hahm et al., Cancer Lett. 2002, 184, 89-96). It is also known that besides curcumin (turmeric), several natural products including resveratrol (peanuts and grape skins) (Manna et al., J. Immunol. 2000, 164, 6509-6519), silymarin (artichoke) (Manna et al., J. Immunol. 1999, 163(12), 6800-6809), oleandrin (Manna et al., Cancer Res. 2000, 60, 3838-3847) and several compounds isolated from both green and black tea leaves (Chung et al., Cancer Res. 1999, 59, 4610-4617) inhibit the AP-1 activation cascade. It is possible that curcumin analogs exhibit their activities on JNK since it is known that both silymarin (Manna et al., J. Immunol. 1999, 163(12), 6800-6809) and oleandrin (Manna et al., Cancer Res. 2000, 60, 3838-3847) inhibit JNK activity.

Because AP-1 and NF-κB responsive genes can promote angiogenesis, cell motility and invasion, and block apoptotic cell death, activation of these genes and their products may result in cancerous or precancerous growth. Therefore, there is a greatly felt need for development of small molecule inhibitors of AP-1 or NF-κB activation.

NF-κB crystal structures are available for use in structure-based drug design including a human NF-κB-DNA structure. However, compounds that have been reported to inhibit activation of NF-κB have generally been suggested or demonstrated to work at the level of IKK, rather than to interfere with NF-κB-DNA interactions or with NF-κB dimerization to prevent its interactions with DNA. For example, it has been shown recently that a new class of retinoid-related anticancer agents inhibits IKK directly. Likewise, a synthetic derivative of the fungal metabolite jesterone, which blocks activation of NF-κB, was shown to specifically inhibit IKKβ.

A number of dietary chemopreventive compounds such as flavonoids and curcumin block activation of NF-κB (Yamamoto et al., J. Clin. Invest. 2001, 107, 135; Bharti et al., Blood 2003, 101, 1053). Curcumin is a non-nutritive, non-toxic compound in turmeric, a spice that has been used for centuries in India and elsewhere as an herbal medicinal treatment of wounds, jaundice, and rheumatoid arthritis (Ammon et al., Planta Med. 1991, 57, 1). In addition, curcumin inhibits the proliferation of a variety of tumor cells and has anti-metastatic activity. Curcumin also exhibits potent anti-oxidant activity, which depends upon the presence of phenolic groups in the aryl rings (Baldwin, A. S. J. Clin. Invest. 2001, 107, 241).

Curcumin is a natural chemoprotective agent that elevates the activities of Phase 2 detoxification enzymes, while inhibiting procarcinogen activating Phase 1 enzymes. It decreases expression of several proto-oncogenes including c-jun, c-fos, and c-myc, and of particular interest, it suppresses the activation of NF-κB. Related to this, curcumin has also been shown to induce apoptosis in several tumor cell lines. In addition to the down-regulation of urokinase-type plasminogen activator (uPA) by dominant negative inhibitors of NF-κB, numerous other factors, including VEGF, IL-8, and MMP-9 that contribute to angiogenesis, invasion, and metastasis, are down-regulated by dominant negative inhibitors of NF-κB. Likewise, curcumin inhibits angiogenesis in vivo.

### SUMMARY OF THE INVENTION

The present invention provides a method of a treating a subject afflicted with cancer or a precancerous condition that includes administering to the subject a therapeutically effective amount of a composition including a compound of Formula I (Ar¹-L-Ar²) wherein L is a divalent linking group that includes an alkylene or an alkenylene including 3, 4, 5, 6, or 7 backbone carbon atoms, wherein one or more of the backbone carbon atoms form part of a carbonyl or secondary alcohol; and Ar¹ and Ar² are each independently aryl groups.

In one embodiment of the invention, either or both of Ar¹ and Ar² are independently heteroaryl groups. In another embodiment, the divalent linking group L is unsaturated. In a further embodiment, L is an alkylene or an alkenylene selected from the group consisting of: —CH═CH—CHO—, —CH═CH—(CO)—CH═CH—, —CH₂—CH₂—(CO)—CH₂CH₂—, —CH₂—CH(OH)—CH₂—CH₂—, 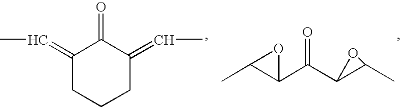
—CH═CH—(CO)—CR═C(OH)—CH═CH—, —CH═CH—(CO)—CR₂—(CO)—CH═CH—, and —CH═CH—(CO)—CH═C(OH)—CH═CH—; wherein R is an alkyl or aryl group including 10 carbon atoms or less.

In a further embodiment of the method of treating a subject afflicted with cancer or a precancerous condition, Ar¹ is a phenyl group according to Formula II: 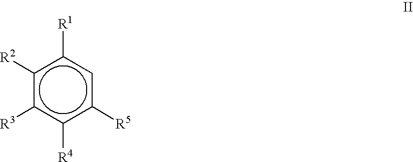
and Ar² is a phenyl group according to Formula III: 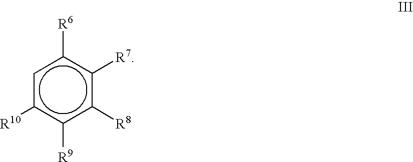
and each of R¹—R¹⁰ is selected from the group consisting of hydrogen, hydroxyl, methyl, methoxyl, dimethylamine, trifluoromethyl, chloro, fluoro, acetoxyl, cyano, and carboxymethyl.

In additional embodiments of the method of the invention, the composition may include a pharmaceutically acceptable carrier. In further embodiments, the composition inhibits AP-1 or NF-κB activity. In some embodiments of the method, the cancer includes tumor cells that constitutively express activated NF-κB, while in additional embodiments the cancer includes tumor cells that constitutively express activated AP-1.

In another aspect, the present invention provides methods for identifying an antitumor curcumin derivative that include contacting a cell including activatable NF-κB with a curcumin derivative; contacting the cell with an NF-κB activator; and determining the effect on NF-κB activation by the curcumin derivative; wherein a curcumin derivative that reduces NF-κB activation is identified as an antitumor curcumin derivative. In embodiments of this aspect of the invention, the NF-κB activator may include TNF-(α or IL-1. In a further embodiment, the cell is a cancer cell. In yet another embodiment, the curcumin derivative is a compound of Formula I (Ar¹-L-Ar²) wherein L is a divalent linking group that includes an alkylene or an alkenylene including 3, 4, 5, 6, or 7 backbone carbon atoms, wherein one or more of the backbone carbon atoms form part of a carbonyl or secondary alcohol; and Ar¹ and Ar² are each independently aryl groups.

In another aspect, the present invention provides methods for identifying an antitumor curcumin derivative that includes contacting a cell including activatable AP-1 with a curcumin derivative; contacting the cell with an AP-1 activator; and determining the effect on AP-1 activation by the curcumin derivative; wherein a curcumin derivative that reduces AP-1 activation is identified as an antitumor curcumin derivative. In embodiments of this aspect of the invention, the AP-1 activator may include TNF-α, PMA, or an MAPK kinase. In a further embodiment, the cell is a cancer cell. In yet another embodiment, the lowering of AP-1 activation by the curcumin derivative occurs by direct inhibition of AP-1 activity, while in another embodiment the lowering of AP-1 activation by the curcumin derivative occurs by indirect inhibition of AP-1 activity. In a further embodiment, the curcumin derivative is a compound of Formula I (Ar¹-L-Ar²) wherein L is a divalent linking group that includes an alkylene or an alkenylene including 3, 4, 5, 6, or 7 backbone carbon atoms, wherein one or more of the backbone carbon atoms form part of a carbonyl or secondary alcohol; and Ar¹ and Ar² are each independently aryl groups.

Another aspect of the invention provides a method of a treating a subject afflicted with cancer or a precancerous condition that includes administering to the subject a therapeutically effective amount of a composition including a compound of Formula IV (Ar¹-L-R¹¹) wherein L is a divalent linking group that includes an alkylene or an alkenylene including 3, 4, 5, 6, or 7 backbone carbon atoms, wherein one or more of the backbone carbon atoms form part of a carbonyl or secondary alcohol; and Ar¹ is an aryl group and R¹¹ is an alkyl group, a heterocyclic group, or a hydrogen.

In one embodiment of this aspect of the invention, one or more of the aryl groups are heteroaryl groups. In a further embodiment, the divalent linking group L is unsaturated, and in another embodiment, R¹¹ is a methyl group. In yet another embodiment, L is an alkylene or an alkenylene selected from the group consisting of: —CH═CH—CHO—, —CH═CH—(CO)—CH═CH—, —CH₂—CH₂—(CO)—CH₂—CH₂—, —CH₂—CH₂—CH(OH)—CH₂—CH₂—, 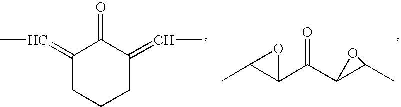
—CH═CH—(CO)—CR═C(OH)—CH═CH—, —CH═CH—(CO)—CR₂—(CO)—CH═CH—, and —CH═CH—(CO)—CH═C(OH)—CH═CH—; wherein R is an alkyl or aryl group including 10 carbon atoms or less.

In a further embodiment of this aspect of the invention, Ar¹ is a phenyl group according to Formula II: 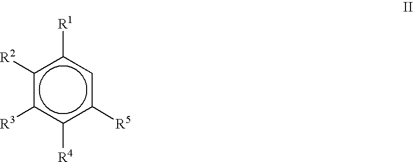
and each of R¹—R⁵ are selected from the group consisting of hydrogen, hydroxyl, methyl, methoxyl, dimethylamine, trifluoromethyl, chloro, fluoro, acetoxyl, cyano, and carboxymethyl.

### BRIEF DESCRIPTION OF THE FIGURES

The patent or application file contains at least one drawing executed in color. Copies of this patent or patent application with color drawings will be provided by the Office upon request and payment of the necessary fee.

FIG. 1 is a pictorial representation of the NF-κB activation cascade.

FIG. 2 is a pictorial representation of the AP-1 activation cascade.

FIG. 3A is a bar graph showing the activities of curcumin analogs including 7-carbon linker groups in the TRAP assay;

FIG. 3B is a bar graph showing the activities of curcumin analogs including 5-carbon linker groups in the TRAP assay;

FIG. 3C is a bar graph showing the activities of curcumin analogs including 3-carbon linker groups in the TRAP assay;

FIG. 4A is a bar graph showing the activities of curcumin analogs including 7-carbon linker groups in the FRAP assay;

FIG. 4B is a bar graph showing the activities of curcumin analogs including 5-carbon linker groups in the FRAP assay;

FIG. 4C is a bar graph showing the activities of curcumin analogs including 3-carbon linker groups in the FRAP assay;

FIG. 5A is a bar graph showing the activities of curcumin analogs including 7-carbon linker groups as inhibitors of the activation of NF-κB by TNFα;

FIG. 5B is a bar graph showing the activities of curcumin analogs including 5-carbon linker groups as inhibitors of the activation of NF-κB by TNFα;

FIG. 5C is a bar graph showing the activities of curcumin analogs including 3-carbon linker groups as inhibitors of the activation of NF-κB by TNFα;

FIG. 6A is a graph showing an IC₅₀ plot of varying doses of curcumin against inhibition of NF-κB activity;

FIG. 6B is a graph showing an IC₅₀ plot of varying doses of analog 31 against inhibition of NF-κB activity;

FIG. 6C is a graph showing an IC₅₀ plot of varying doses of analog 29 against inhibition of NF-κB activity;

FIG. 6D is a graph showing an IC₅₀ plot of varying doses of analog 38a against inhibition of NF-κB activity;

FIG. 6E is a graph showing an IC₅₀ plot of varying doses of analog 20q against inhibition of NF-κB activity;

FIG. 6F is a graph showing an IC₅₀ plot of varying doses of analog 38a against inhibition of NF-κB activity;

FIG. 6G is a graph showing an IC₅₀ plot of varying doses of analog 20ag against inhibition of NF-κB activity;

FIG. 6H is a graph showing an IC₅₀ plot of varying doses of analog 20m against inhibition of NF-κB activity;

FIG. 6I is a graph showing an IC₅₀ plot of varying doses of analog 6a against inhibition of NF-κB activity;

FIG. 6J is a graph showing an IC₅₀ plot of varying doses of analog 20v against inhibition of NF-κB activity;

FIG. 6K is a graph showing an IC₅₀ plot of varying doses of analog 9a against inhibition of NF-κB activity;

FIG. 6L is a graph showing an IC₅₀ plot of varying doses of analog 20a against inhibition of NF-κB activity;

FIG. 7 is a computer-generated image representing NF-κB (1IKN) bound to IκB;

FIG. 8 is a computer-generated image representing NF-κB (1IKN) with IκB removed;

FIG. 9 is a computer-generated image representing the front face of NF-κB (1IKN) with bound analogs;

FIG. 10 is a computer-generated image representing curcumin bound to NF-κB (1IKN);

FIG. 11 is a computer-generated image representing the opposite face of NF-κB (1IKN) with bound analogs;

FIG. 12 is a computer-generated image representing NF-κB (1IKN) with MES and bound analogs;

FIG. 13 is a computer-generated image representing curcumin bound to NF-κB (1IKN) with MES;

FIG. 14 is a computer-generated image representing NF-κB (1SVC) bound to DNA;

FIG. 15 is a computer-generated image representing NF-κB (1SVC) with DNA removed;

FIG. 16 is a computer-generated image representing NF-κB (1SVC) with bound analogs;

FIG. 17 is a computer-generated image representing curcumin bound to NF-κB (1SVC);

FIG. 18 is a computer-generated image representing the opposite face of NF-κB (1SVC) with bound analogs; and

FIG. 19 is a computer-generated image representing NF-κB (1SVC) with MES and bound analogs.

FIG. 20A is a bar graph showing the activities of curcumrin analogs including 7-carbon analogs active in the AP-1 assay.

FIG. 20B is a bar graph showing the activities of curcumin analogs including 5-carbon analogs active in the AP-1 assay.

FIG. 20C is a bar graph showing the activities of curcumin analogs including 3-carbon analogs active in the AP-1 assay.

FIG. 21A is a bar graph showing the activities of curcumin analogs including 7-carbon analogues in the AP-1 assay.

FIG. 21B is a bar graph showing the activities of curcumin analogs including 5-carbon analogues in the AP-1 assay.

FIG. 21C is a bar graph showing the activities of curcumin analogs including 3-carbon analogues in the AP-1 assay.

FIG. 22A is a graph showing an IC₅₀ plot of varying doses of analog 20m against inhibition of AP-1 activity;

FIG. 22B is a graph showing an IC₅₀ plot of varying doses of analog 31 against inhibition of AP-1 activity;

FIG. 22C is a graph showing an IC₅₀ plot of varying doses of analog 20o against inhibition of AP-1 activity;

FIG. 22D is a graph showing an IC₅₀ plot of varying doses of analog 9a against inhibition of AP-1 activity;

FIG. 22E is a graph showing an IC₅₀ plot of varying doses of analog 6a against inhibition of AP-1 activity;

FIG. 22F is a graph showing an IC₅₀ plot of varying doses of analog 20d against inhibition of AP-1 activity;

FIG. 22G is a graph showing an IC₅₀ plot of varying doses of analog 20c against inhibition of AP-1 activity;

FIG. 22H is a graph showing an IC₅₀ plot of varying doses of analog 38a against inhibition of AP-1 activity;

FIG. 22I is a graph showing an IC₅₀ plot of varying doses of analog 29 against inhibition of AP-1 activity;

FIG. 22J is a graph showing an IC₅₀ plot of varying doses of analog 20ag against inhibition of AP-1 activity;

FIG. 22K is a graph showing an IC₅₀ plot of varying doses of analog 20q against inhibition of AP-1 activity;

FIG. 22L is a graph showing an IC₅₀ plot of varying doses of curcumin against inhibition of AP-1 activity;

FIG. 23 is a computer-generated image representing AP-1 bound to DNA.

FIG. 24 is a computer-generated image representing AP-1 with DNA removed.

FIG. 25 is a computer-generated image representing the front face of AP-1 with analogs.

FIG. 26 is a computer-generated image representing the opposite face of AP-1 with analogs.

FIG. 27 is a computer-generated image representing AP-1 with MES and analogs.

FIG. 28 is a computer generated pharmacophore model with the structure of curcumin superimposed on the model.

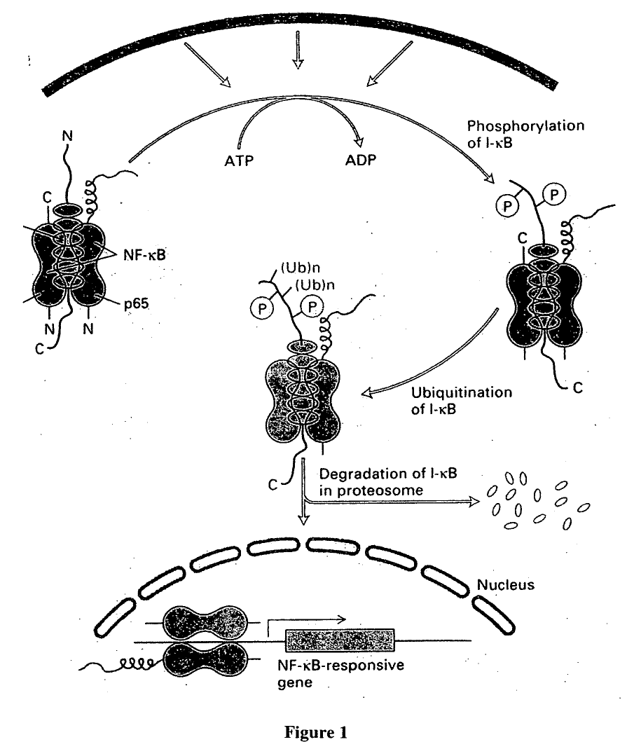

### DETAILED DESCRIPTION OF ILLUSTRATIVE EMBODIMENTS OF THE INVENTION

Definitions

Unless otherwise specified, “a,” “an,” “the,” and “at least one” are used interchangeably and mean one or more than one.

The terminology as set forth herein is for description of the embodiments only and should not be construed as limiting of the invention as a whole. As used in the description of the invention and the appended claims, the singular forms “a”, “an”, and “the” are inclusive of their plural forms, unless contraindicated by the context surrounding such.

The terms “comprising” and variations thereof do not have a limiting meaning where these terms appear in the description and claims.

As used herein, the term “organic group” is used for the purpose of this invention to mean a hydrocarbon group that is classified as an aliphatic group, cyclic group, or combination of aliphatic and cyclic groups (e.g., alkaryl and aralkyl groups). In the context of the present invention, suitable organic groups for curcumin derivatives of this invention are those that do not interfere with the curcumin derivatives' antitumor activity. In the context of the present invention, the term “aliphatic group” means a saturated or unsaturated linear or branched hydrocarbon group. This term is used to encompass alkyl, alkenyl, and alkynyl groups, for example.

As used herein, the terms “alkyl”, “alkenyl”, and the prefix “alk-” are inclusive of straight chain groups and branched chain groups and cyclic groups, e.g., cycloalkyl and cycloalkenyl. Unless otherwise specified, these groups contain from 1 to 20 carbon atoms, with alkenyl groups containing from 2 to 20 carbon atoms. In some embodiments, these groups have a total of at most 10 carbon atoms, at most 8 carbon atoms, at most 6 carbon atoms, or at most 4 carbon atoms. Cyclic groups can be monocyclic or polycyclic and preferably have from 3 to 10 ring carbon atoms. Exemplary cyclic groups include cyclopropyl, cyclopropylmethyl, cyclopentyl, cyclohexyl, adamantyl, and substituted and unsubstituted bornyl, norbornyl, and norbornenyl.

The term “heterocyclic” includes cycloalkyl or cycloalkenyl non-aromatic rings or ring systems that contain at least one ring heteroatom (e.g., O, S, N).

Unless otherwise specified, “alkylene” and “alkenylene” are the divalent forms of the “alkyl” and “alkenyl” groups defined above. The terms, “alkylenyl” and “alkenylenyl” are used when “alkylene” and “alkenylene”, respectively, are substituted. For example, an arylalkylenyl group comprises an alkylene moiety to which an aryl group is attached.

The term “haloalkyl” is inclusive of groups that are substituted by one or more halogen atoms, including perfluorinated groups. This is also true of other groups that include the prefix “halo-”. Examples of suitable haloalkyl groups are chloromethyl, trifluoromethyl, and the like. Halogens are elements including chlorine, bromine, fluorine, and iodine.

The term “aryl” as used herein includes monocyclic or polycyclic aromatic hydrocarbons or ring systems. Examples of aryl groups include phenyl, naphthyl, biphenyl, fluorenyl and indenyl. Aryl groups may be substituted or unsubstituted. Aryl groups include aromatic annulenes, fused aryl groups, and heteroaryl groups. Aryl groups are also referred to herein as aryl rings.

Unless otherwise indicated, the term “heteroatom” refers to the atoms O, S, or N.

The term “heteroaryl” includes aromatic rings or ring systems that contain at least one ring heteroatom (e.g., O, S, N). In some embodiments, the term “heteroaryl” includes a ring or ring system that contains 2 to 12 carbon atoms, 1 to 3 rings, 1 to 4 heteroatoms, and O, S, and/or N as the heteroatoms. Suitable heteroaryl groups include furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, pyrazinyl, 1-oxidopyridyl, pyridazinyl, triazinyl, tetrazinyl, oxadiazolyl, thiadiazolyl, and so on.

The terms “arylene” and “heteroarylene” are the divalent forms of the “aryl” and “heteroaryl” groups defined above. The terms “arylenyl” and “heteroarylenyl” are used when “arylene” and “heteroarylene”, respectively, are substituted. For example, an alkylarylenyl group comprises an arylene moiety to which an alkyl group is attached.

The term “fused aryl ring” includes fused carbocyclic aromatic rings or ring systems. Examples of fused aryl rings include benzo, naphtho, fluoreno, and indeno.

The term “annulene” refers to aryl groups that are completely conjugated monocyclic hydrocarbons. Annulenes have a general formula of CₙHₙ, where n is an even number, or CₙHₙ₊₁, where n is an odd number. Examples of annulenes include cyclobutadiene, benzene, and cyclooctatetraene. Annulenes present in an aryl group will typically have one or more hydrogen atoms substituted with other atoms such as carbon.

When a group is present more than once in any formula or scheme described herein, each group (or substituent) is independently selected, whether explicitly stated or not. For example, for the formula —C(O)—NR₂ each of the two R groups is independently selected.

As a means of simplifying the discussion and the recitation of certain terminology used throughout this application, the terms “group” and “moiety” are used to differentiate between chemical species that allow for substitution or that may be substituted and those that, in the particular embodiment of the invention, do not so allow for substitution or may not be so substituted. Thus, when the term “group” is used to describe a chemical substituent, the described chemical material includes the unsubstituted group and that group with nonperoxidic O, N, S, Si, or F atoms, for example, in the chain as well as carbonyl groups or other conventional substituents. Where the term “moiety” is used to describe a chemical compound or substituent, only an unsubstituted chemical material is intended to be included. For example, the phrase “alkyl group” is intended to include not only pure open chain saturated hydrocarbon alkyl substituents, such as methyl, ethyl, propyl, tert-butyl, and the like, but also alkyl substituents bearing further substituents known in the art, such as hydroxy, alkoxy, alkylsulfonyl, halogen atoms, cyano, nitro, amino, carboxyl, etc. Thus, “alkyl group” includes ether groups, haloalkyls, nitroalkyls, carboxyalkyls, hydroxyalkyls, sulfoalkyls, etc. On the other hand, the phrase “alkyl moiety” is limited to the inclusion of only pure open chain saturated hydrocarbon alkyl substituents, such as methyl, ethyl, propyl, tert-butyl, and the like.

The invention is inclusive of the compounds described herein (including intermediates) in any of their pharmaceutically acceptable forms, including isomers (e.g., diastereomers and enantiomers), tautomers, salts, solvates, polymorphs, prodrugs, and the like. In particular, if a compound is optically active, the invention specifically includes each of the compound's enantiomers as well as racemic mixtures of the enantiomers. It should be understood that the term “compound” includes any or all of such forms, whether explicitly stated or not (although at times, “salts” are explicitly stated).

“Treat”, “treating”, and “treatment”, etc., as used herein, refer to any action providing a benefit to a patient at risk for or afflicted with a disease, including improvement in the condition through lessening or suppression of at least one symptom, delay in progression of the disease, prevention or delay in the onset of the disease, etc.

Treatment, as used herein, encompasses both prophylactic and therapeutic treatment. Curcumin derivatives of the invention can, for example, be administered prophylactically to a mammal in advance of the occurrence of cancer. Prophylactic administration is effective to decrease the likelihood of the subsequent occurrence of cancer in the mammal, or decrease the severity of cancer that subsequently occurs. Alternatively, curcumin derivatives of the invention can, for example, be administered therapeutically to a mammal that is already afflicted by cancer or a precancerous condition. In one embodiment of therapeutic administration, administration of the curcumin derivatives are effective to eliminate the cancer or precancerous condition; in another embodiment, administration of the curcumin derivatives is effective to decrease the severity of the cancer or precancerous condition or lengthen the lifespan of the mammal so afflicted.

“Pharmaceutically acceptable” as used herein means that the compound or composition is suitable for administration to a subject to achieve the treatments described herein, without unduly deleterious side effects in light of the severity of the disease and necessity of the treatment.

“Inhibit” as used herein refers to the partial or complete elimination of a potential effect, while inhibitors are compounds that have the ability to inhibit.

The present invention provides methods for the use of curcumin derivatives to treat or prevent cancer or a precancerous condition in a subject. The present invention also provides methods for identifying and preparing curcumin derivatives that may be used to treat or prevent cancer or a precancerous condition in a subject.

Curcumin (diferuloylmethane, 1,7-bis(4-hydroxy-3-methoxyphenyl)-1,6-heptadiene-3,5-dione) is a symmetrical diphenolic dienone. It exists in solution as an equilibrium mixture of the symmetrical dienone (diketo) and the keto-enol tautomer; the keto-enol form is strongly favored by intramolecular hydrogen bonding.

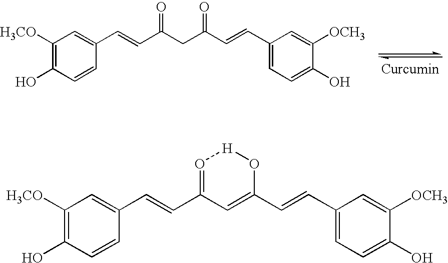

Curcumin contains two aryl rings separated by an unsaturated seven carbon spacer having two carbonyls. The aryl rings of curcumin contain a hydroxyl group in the para position and a methoxy group in the meta position.

Curcumin Derivatives

Curcumin derivatives are expected to be beneficial for use in the prescribed antitumor activity. The term “curcumin derivatives,” as used herein, includes, for example, curcumin analogs, curcuminoids and chalcones. In one embodiment, the curcumin derivative includes first and second aryl groups covalently attached by way of a spacer, also referred to herein as a linker or a linking group. In another embodiment, the second aryl group is absent, such that the curcumin derivative contains a first aryl group and the spacer but no second aryl group at the distal end of the spacer. Optionally, the first and/or second aryl group is a heteroaryl group. The first and second aryl groups may be independently substituted or unsubstituted.

Representative curcumin derivatives are described herein, and also in Weber et al., Bioorg. Med. Chem. 13 (2005) 3811-3820; Weber et al., Bioorg. Med. Chem. (2005), published online on Dec. 7, 2005, and retrieved from www.sciencedirect.com, and US Pat. Publ. 2001-0051184 A1, published Dec. 13, 2001 (Heng).

Curcumin derivatives that exhibit improved pharmacokinetic properties and/or reduced toxicity are preferred. For example, curcumin derivatives that include heteroaryl groups and/or unsaturated spacers are expected to impart improved pharmacokinetic properties and/or reduced toxicity to the compounds, because they are expected to be less chemically reactive in vivo. Such derivatives are expected to be less likely to be degraded and/or form toxic adducts or intermediates under physiological conditions. Additional curcumin derivatives not encompassed by the general definition provided above may also be found in the examples and schemes provided herein.

Curcumin derivatives of the invention are generally encompassed by Formula I:
Ar¹-L-Ar² (I)
wherein Ar² is optional; L is a divalent linking group comprising an alkylene or an alkenylene that includes between 3 and 7 backbone carbon atoms, wherein one or more of the backbone carbon atoms include a carbonyl or hydroxyl moiety; and Ar¹ and Ar² (if Ar² is present) are independently aryl groups. Ar¹ and Ar² (if Ar² is present) may be unsubstituted or may optionally include one or more substituents selected from the group consisting of hydroxyl, alkyl, alkenyl, haloalkyl, alkoxy, and NR₂, where R is hydrogen or alkyl. If Ar² is absent, it may be replaced by a substituent R¹¹, including hydrogen (H). R¹¹ can be, for example, a heterocyclic group or an alkyl group, preferably an alkyl group having four or fewer carbon atoms, e.g., a methyl group. R¹¹ can alternately be an amine, a hydroxyl, or a hydrogen.
Aryl Groups

Curcumin derivatives of the invention include aryl group Ar¹, which is positioned at an end of the linker L. Curcumin derivatives of the invention may optionally include a second aryl group Ar² that is independently selected from Ar¹, which is positioned at the other end of the linker L relative to Ar¹ when present. Preferred aryl groups include phenyl groups, naphthyl groups, thienyl groups, and pyridinium groups.

Aryl groups Ar¹ and Ar² may be substituted or unsubstituted. Preferably, substituents are selected from the group consisting of hydroxyl, halogen, alkyl, alkenyl, haloalkyl, alkoxy, amine, carboxyl, and ester substituents.

For example, in one embodiment of the invention, Ar¹ can be a phenyl group according to Formula II: 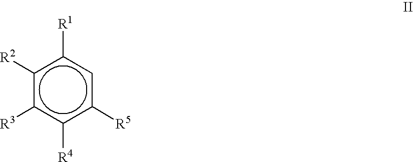
and Ar² can be a phenyl group according to Formula III: 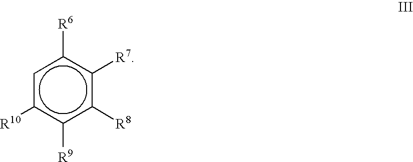
The ring positions may, independently, be unsubstituted (i.e., R=hydrogen) or one or more R groups may be substituents independently selected from a variety of substituents, including hydroxyl, halogen, alkyl, alkenyl, haloalkyl, alkoxy, amine, carboxyl, and ester substituents. In further embodiments, R¹—R¹⁰ are each independently selected from the group including hydrogen (—H), hydroxyl (—OH), methyl (—CH₃), methoxyl (—OCH₃), dimethylamine (—N(CH₃)₂), chloro (—Cl), fluoro (—F), trifluoromethyl (—CF₃), acetoxyl, (—O(CO)CH₃) and carboxymethyl (—C(CO)OCH₃) moieties.
Divalent Linking Groups

The linker L is a spacer that preferably includes 3, 4, 5, 6 or 7 carbon atoms that form a linear carbon chain connecting the first and second aryl groups. The carbons atoms in the carbon chain that trace out shortest path between the first and optional second aryl groups are referred to herein as the “backbone” carbon atoms. The number of backbone carbon atoms is readily determined in straight chain alkyl groups. In spacers that include a cyclic alkyl group as a constituent of the linear chain (e.g., 38a), the backbone carbon atoms include the least number of ring carbons possible, e.g., 3 ring carbons in 38a. The number of backbone carbon atoms is used herein as a shorthand way to designate the length of the linker being used. For example, a 7-carbon spacer is a divalent spacer that includes 7 backbone carbon atoms. Preferred embodiments of the invention include curcumin derivatives having an odd number of carbon atoms; e.g., 3, 5, and 7-carbon linking groups.

Preferably at least one of the backbone carbon atoms is included in a carbonyl (C═O) moiety. The spacer may be substituted or unsubstituted. The spacer may further be saturated or unsaturated. In a preferred embodiment, the spacer contains an odd number of carbon atoms (i.e., 3, 5, or 7 carbon atoms), and at least one unsaturated carbon-carbon bond. In additional embodiments, the spacer may include a hydroxyl moiety in place of, or in addition to, the at least one carbonyl moiety.

Curcumin derivatives of the invention include a linking group L that is preferably covalently attached at one end to aryl group Ar¹. Optionally, the linking group L may also be covalently attached at the other end to a second aryl group, Ar², which is selected independently from Ar¹. The linking group L is a divalent linking group that preferably includes an alkylene or an alkenylene group having between 3 and 7 backbone carbon atoms and preferably at least one carbonyl moiety. The linking group may be substituted or unsubstituted, and may be saturated or unsaturated. Preferably, an unsaturated linking group includes conjugated double bonds. Preferably the linking group also contains an odd number of carbon atoms (i.e., 3, 5, or 7 carbon atoms), and at least one unsaturated carbon-carbon bond. In additional embodiments, the linking group may include a hydroxyl moiety in place of, or in addition to, the at least one carbonyl moiety. Table 1 shows compounds with 7-carbon linkers; Table 2 shows compounds with 5-carbon linkers; and Table 3 shows compounds with 3-carbon linkers.

A divalent linking group includes two carbons with unfilled valencies that provide valence points where a covalent bond can be formed to an adjacent alkyl or aryl group that also includes a carbon with an unfilled valency. Generally, a valence point is represented in a chemical formula by a bond that is shown as not being attached to another group (e.g., CH₃—, wherein —0 represents the valence point). In embodiments wherein the curcumin derivative lacks the second aryl group Ar², the distal valence point on the linking group can be filled with any substituent of interest, preferably a short chain alkyl group or a hydrogen (H). Compounds lacking a second aryl group may be represented by Formula IV:
Ar¹-L-R¹¹ (IV)
R¹¹ in Formula IV can be, for example, a heterocyclic group or an alkyl group, preferably an alkyl group having four or fewer carbon atoms, e.g., a methyl group. R¹¹ can alternately be an amine, a hydroxyl, or a hydrogen.
Curcumin Derivatives Including 7-Carbon Linking Groups

In one embodiment of the invention, the curcumin derivatives include one or two aryl groups (Ar¹ and optionally Ar²) and a linking group L that is a 7-carbon linking group (i.e., a linking group that includes 7 backbone carbon atoms). Preferably, the 7-carbon linking group includes at least one unsaturated carbon-carbon bond. Examples of 7-carbon linking groups include
—CH═CH—(CO)—CR═C(OH)—CH═CH—,
—CH═CH—(CO)—CR₂—(CO)—CH═CH—, and
—CH═CH—(CO)—CH═C(OH)—CH═CH—.

where R includes substituent alkyl or aryl groups comprising 10 carbon atoms or less. In some embodiments, R may be a methyl, ethyl, or benzyl group. These linking groups are the divalent forms of 4-alkyl-1,6 heptadiene-3,5-dione; 4,4-dialkyl-1,6 heptadiene-3,5-dione; and heptane-3,5-dione.

Examples of 7-C Linkers

Table 1 shows a number of examples of curcumin derivatives that include a seven carbon linker. The compounds shown contain two aryl rings separated by a seven carbon spacer having two carbonyls (or the equivalent keto-enol tautomer). In many, but not all, of the compounds, the spacer is unsaturated.

TABLE 1
7-Carbon Linker Analogs.

| | |
|---|---|
| 3a | 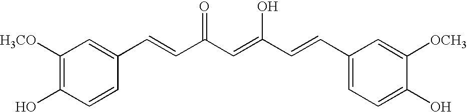 |
| 3b | 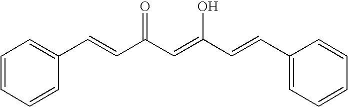 |
| 3c | 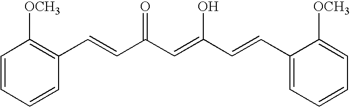 |
| 3d | 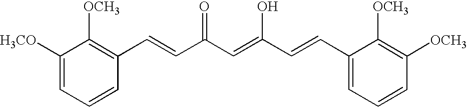 |
| 3e | 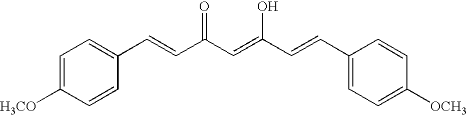 |
| 3f | 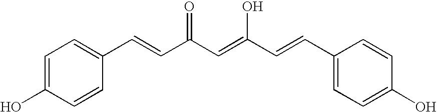 |
| 3g | 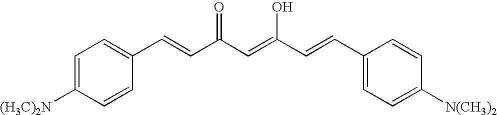 |
| 3h | 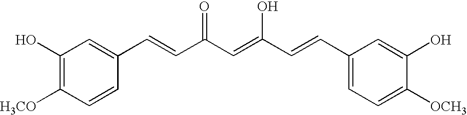 |
| 3i | 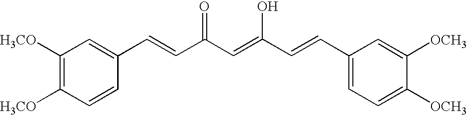 |
| 6a | 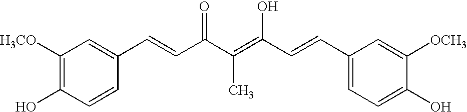 |
| 6b | 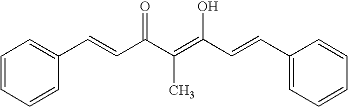 |
| 9a | 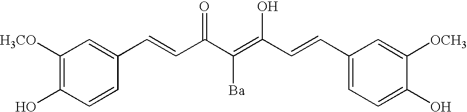 |
| 9b | 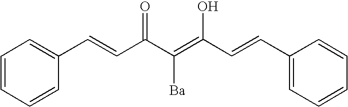 |
| 11b | 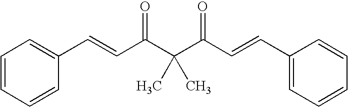 |
| 12b | 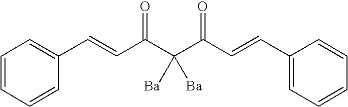 |
| 13a | 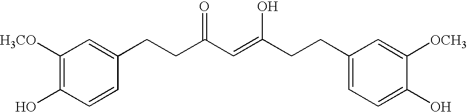 |
| 13b | 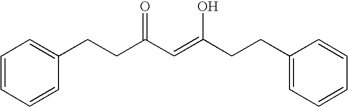 |
| 14a | 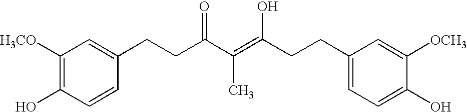 |
| 14b | 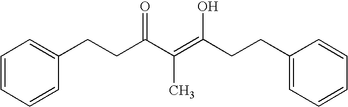 |
| 15a | 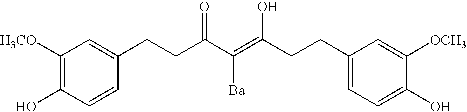 |
| 15b | 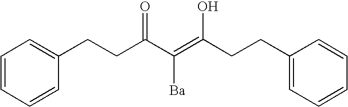 |
| 16b | 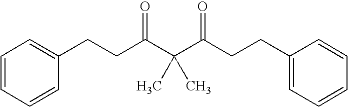 |
| 17b | 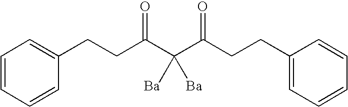 |

Curcumin Derivatives Including 5-Carbon Linking Groups

In a further embodiment of the invention, the curcumin derivatives include one or two aryl groups (Ar¹ and optionally Ar²) that are linked by a linking group L that is a 5-carbon linking group (i.e., a linking group that includes 5 backbone carbon atoms). Preferably, the 5-carbon linking group includes at least one unsaturated carbon-carbon bond. Examples of 5-carbon linking groups include:

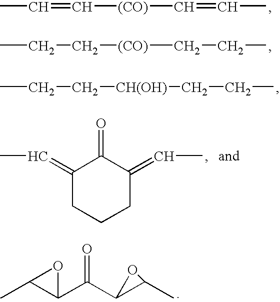

These linking groups are the divalent forms of 1,4-pentadiene-3-one; pentan-3-one; pentan-3-ol, 2,6;bis(methylene)cyclohexanone; and 1,2,4,5-diepoxy pentan-3-one. As noted herein, curcumin derivatives may include a cyclic linking group. For example, compound 31 (1-methyl-2,6-diphenyl-4-piperidone), provided in Example 4 herein, provides a compound with a 5-carbon linking group that is bridged by a tertiary amine to form a cyclic alkylene linking group including the heteroatom nitrogen.

Examples of 5-C Linkers

Table 2 shows a number of examples of curcumin derivatives that include a five carbon linker. The compounds shown contain two aryl rings separated by a five carbon spacer having a single carbonyl or hydroxyl. In many, but not all, of the compounds, the spacer is unsaturated.

TABLE 2
5-Carbon Linker Analogs.

| | |
|---|---|
| 20a | 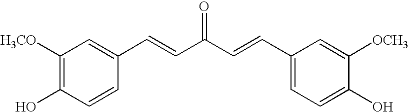 |
| 20b | 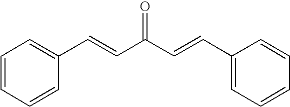 |
| 20c | 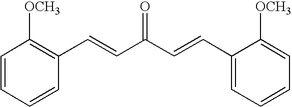 |
| 20d | 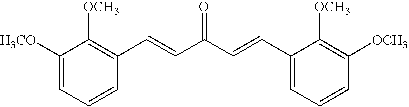 |
| 20e | 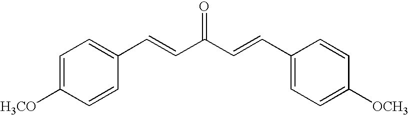 |
| 20f | 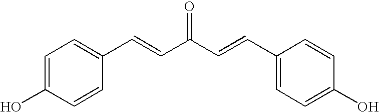 |
| 20g | 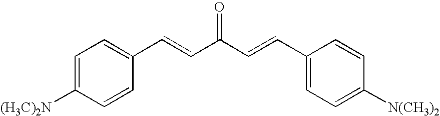 |
| 20i | 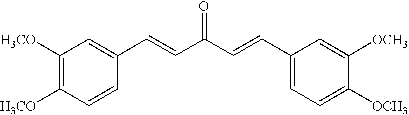 |
| 20k | 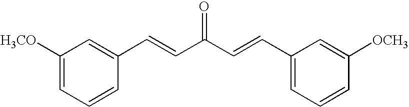 |
| 20l | 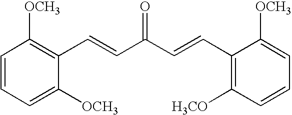 |
| 20m | 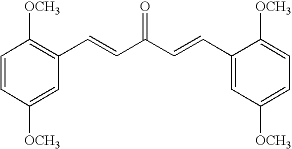 |
| 20n | 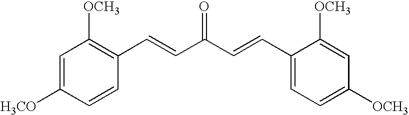 |
| 20o | 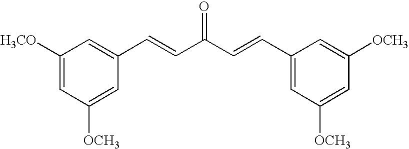 |
| 20p | 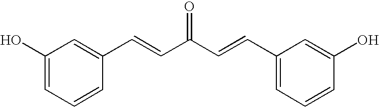 |
| 20q | 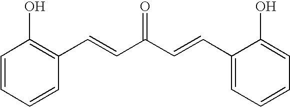 |
| 20r | 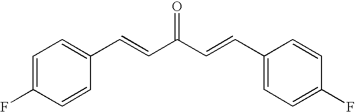 |
| 20s | 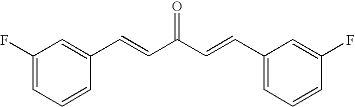 |
| 20t | 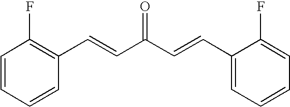 |
| 20u | 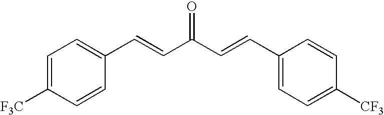 |
| 20v | 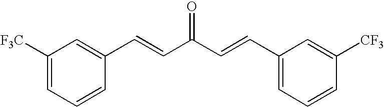 |
| 20w | 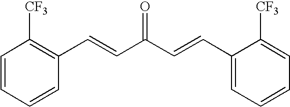 |
| 20x | 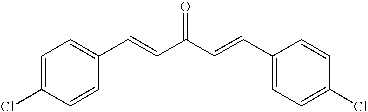 |
| 20y | 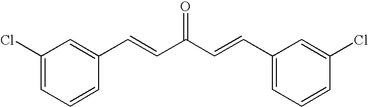 |
| 20z | 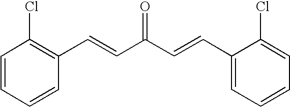 |
| 20aa | 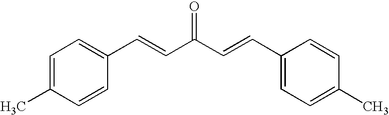 |
| 20ab | 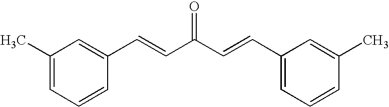 |
| 20ac | 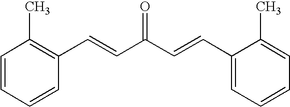 |
| 20ae | 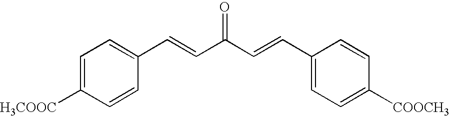 |
| 20af | 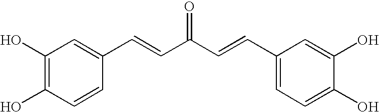 |
| 20ag | 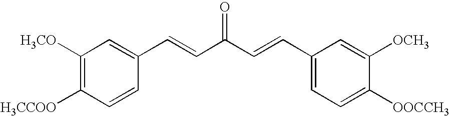 |
| 20ah | 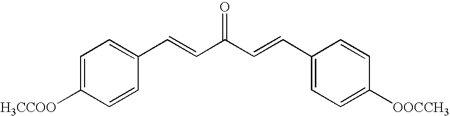 |
| 23 | 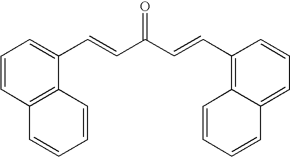 |
| 25 | 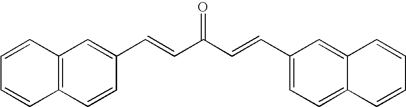 |
| 29 | 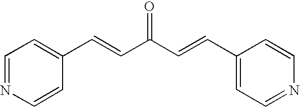 |
| 31 | 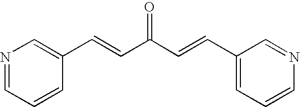 |
| 34 | 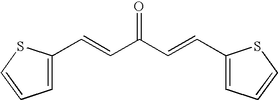 |
| 36a | 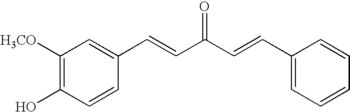 |
| 36e | 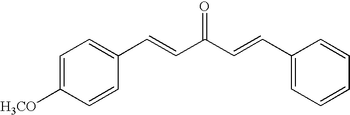 |
| 38a | 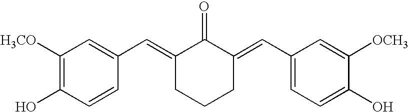 |
| 38b | 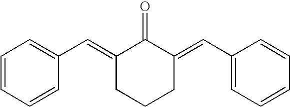 |
| 39b | 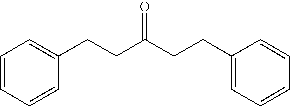 |
| 40b | 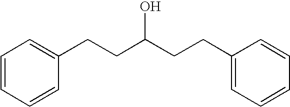 |
| 42b | 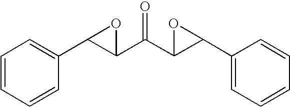 |
| 43b | 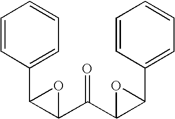 |

Curcumin Derivatives Including 3Carbon Linking Groups

In a further embodiment of the invention, the curcumin derivatives include one or two aryl groups (Ar¹ and optionally Ar²) that are linked by a linking group L that is a 3-carbon linking group (i.e., a linking group that includes 3 backbone carbon atoms). Preferably, the 3-carbon linking group includes at least one unsaturated carbon-carbon bond. An example of a 3-carbon linking group is —CH═CH—CH(O)—; i.e., a divalent form of propenone.

Examples of 3-C Linkers

Table 3 shows a number of examples of curcumin derivatives that include a three carbon linker. The compounds shown generally have an unsaturated three-carbon spacer having a single carbonyl. While most of the examples shown have two aryl groups seperated by the spacer, several of the embodiments include only a single aryl group. In the examples that include only a single aryl group, a methyl group is provided at the other end of the linking group. Compound 52b includes the heteroatom N in place of one of the backbone carbon atoms; however, this is still considered a 3-C linker in that 3 atoms (C, N, and C) are present along the shortest bridge between the two aryl groups.

TABLE 3
3-Carbon Linker Analogs.

| | |
|---|---|
| 35a | 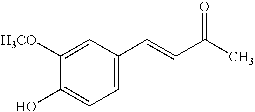 |
| 35e | 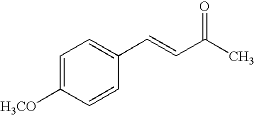 |
| 35q | 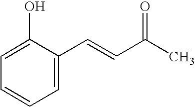 |
| 45a | 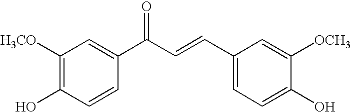 |
| 45b | 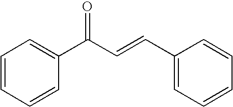 |
| 46a | 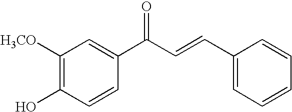 |
| 46ad | 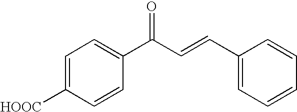 |
| 46ak | 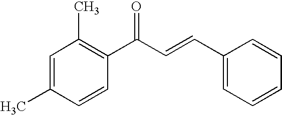 |
| 46al | 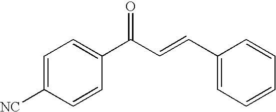 |
| 48a | 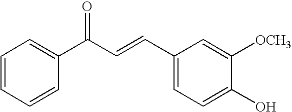 |
| 48ad | 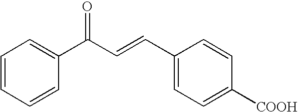 |
| 50b | 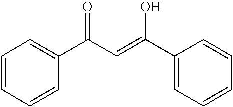 |
| 52b | 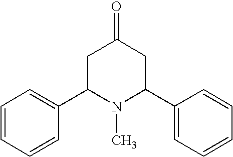 |

Additional Curcumin Derivatives

Curcumin derivatives of the invention may include a variety of linking groups and Ar groups while retaining antitumor activity, so long as they provide a structure that will inhibit NK-κB or AP-1 activity. Accordingly, additional curcumin analogs are contemplated. Since analogs that contain a central methylene substituent on the 7-carbon spacer have shown significant activity, analogs containing a central group other than methyl or benzyl may also exhibit significant inhibition of NF-κB and/or AP-1 activation. These include curcumin analogs containing central methylene substituents such as ethyl, propyl, butyl, isopropyl and substituted benzyl groups as shown below:

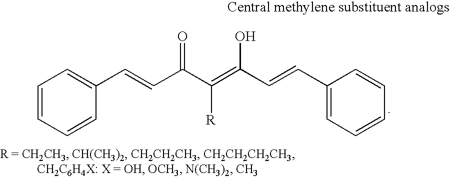

These compounds can be synthesized using the procedures shown in Schemes 1 and Scheme 2. The descriptions and details for these procedures are the same as those described for Schemes 8 and 9.

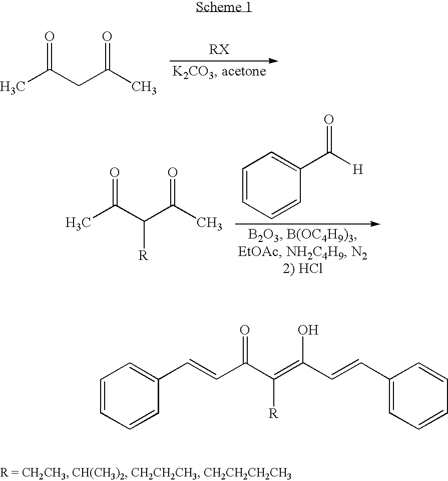

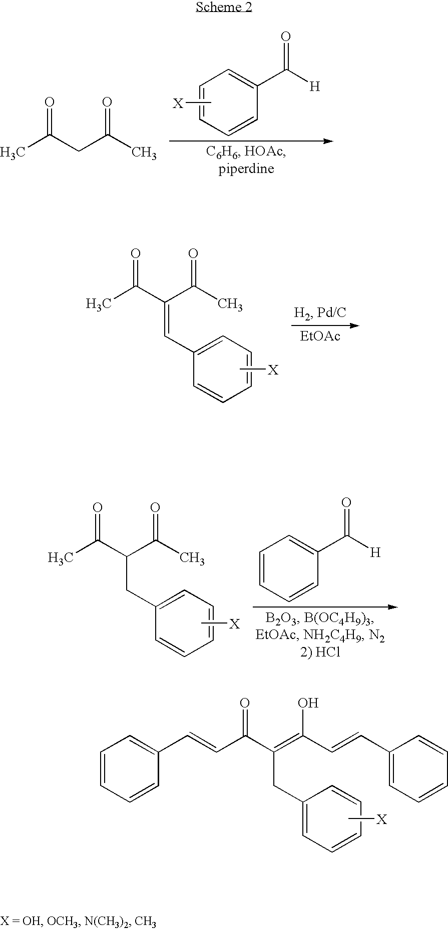

Additional analogs that are contemplated are those having a pyridine ring with and without a central methylene substituent on the 7-carbon spacer such as those shown below. Analogs without a central methylene substituent can be prepared according to Pabon's method shown in Scheme 3. The descriptions and details for this procedure are the same as described for Scheme 6. The analogs having a pyridine aryl ring with a central methylene substituent on the 7-carbon spacer can be synthesized using a procedure described in Scheme 1 using 2, 3 or 4-pyridine carboxaldehyde.

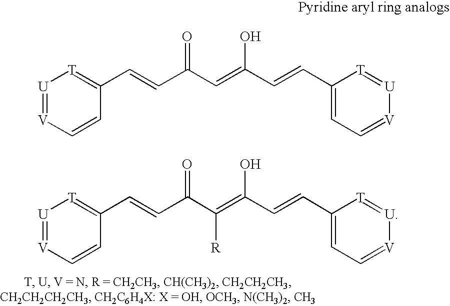

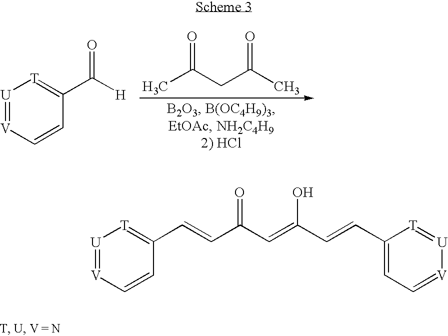

Many curcumin analogs which have a 5-carbon spacer possess significant activity. Additional active analogs in this series may contain substituents such as hydroxy and methoxy groups on the aryl rings. Therefore, other substituents and their positions on the aryl rings may also provide significant inhibition of NF-κB and/or AP-1 activation. Examples of these analogs are shown below: 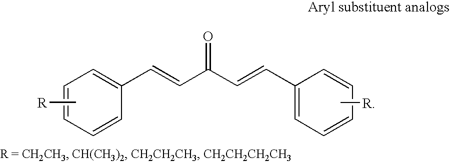
These new analogs can be prepared as shown in Scheme 4. The descriptions and details for this procedure are the same as described for Scheme 13. 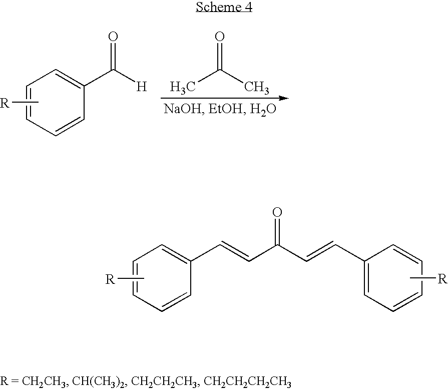
Although analogs having 3-carbon spacers were generally not as active as analogs having 7-carbon or 5-carbon spacers, additional analogs may provide significant inhibition of NF-κB and/or AP-1 activation. Analogs having different substituents on the aryl rings may provide significant inhibition of NF-κB and/or AP-1 activation. In addition, analogs that contain different substituents on the nitrogen of the heterocyclic ring may provide significant inhibition of NF-κB and/or AP-1 activation. Examples of these series 3 analogs are shown below: 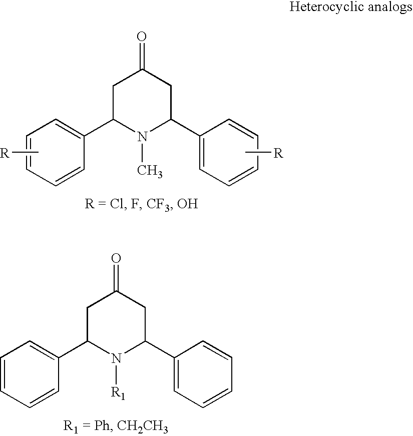
These analogs can be synthesized as shown in Scheme 5. The descriptions and details for this procedure are the same as those described for Scheme 36. 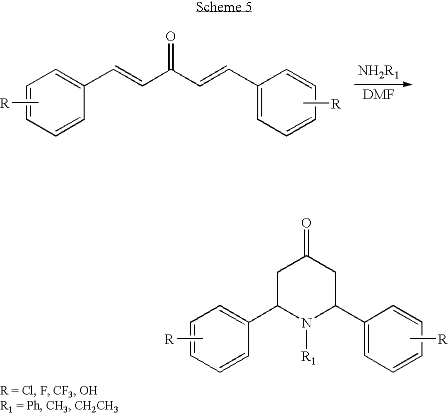
Additional curcumin derivatives of the invention that are not encompassed by the embodiments provided above may also be found in the examples and schemes provided herein.
Cancer Treatment Using Curcumin Derivatives

The present invention provides methods for treating a subject with cancer or a precancerous condition by administering to the subject a curcumin derivative as described herein. Without being bound by theory, it is believed that administration of the curcumin derivative inhibits the activity of AP-1 and/or NF-κB. The literature on the anti-cancer properties of curcumin includes reports of direct inhibition by curcumin of enzymes that may be important in cancer progression, such as inhibition of c-Jun N-terminal kinase, (Du, et al., J. Cell. Biochem. 77, 333 (2000)) epidermal growth factor receptor, (Korutia, et al., Carcinogenesis 16, 1741 (1995)) and p185neu (Hong, et al., Clin. Cancer Res. 5, 1884 (1999)). The pro-apoptotic activity of curcumin in many types of cancer cells, where the classical hallmarks of apoptosis are observed including DNA laddering, chromatin condensation, and cleavage of 28S and 18S ribosomal RNA, (Jiang, et al., Nutr. Cancer 26, 111 (1996)) may be related to the activation of NF-κB since many cancer cells protect against apoptosis by activating NF-κB as a pro-survival strategy. Over-expression of p65 renders cells resistant to the pro-apoptotic effects of curcumin. Anto, et al., J. Biol. Chem. 275, 15601 (2000). When these cells are then transiently transfected with a super-repressor form of IκBa, these cells are no longer resistant to curcumin, consistent with an important role for NF-κB in the apoptosis-inducing activity of curcumin. Studies designed to identify the specific target(s) of curcumin in preventing the activation of NF-κB point toward targets that are upstream from I-κB. Singh and Aggarwal reported that curcumin inhibited TNFα-dependent activation of NF-κB at a step before phosphorylation of I-κB but after a point where multiple stimuli converge. Singh, S.; Aggarwal, B. B. J. Biol. Chem. 270, 24995 (1995). Brennan and O'Neill reported that curcumin inhibited degradation of I-κB but also reacted with p50. Brennan, P.; O'Neill, L. A. Biochem. Pharmacol. 55, 965 (1998). Several groups have reported that curcumin inhibited IKK or that curcumin targets a kinase that is upstream from IKK. Jobin et al., J. Immunol. 163, 3474 (1999); Plummer et al., Oncogene 18, 6013 (1999). The IKK complex includes IKKa, IKKb, IKKc/NEMO, as well as the I-κB recruiter/regulator ELKS, Sigala et al., Science 304, 1963 (2004), and the chaperone HSP90 and co-chaperone Cdc37, Chen et al., Mol. Cell 9, 401 (2002). More recently, c-Src has been reported to be part of the IKK complex. Funakoshi-Tago et al., J. Biochem. (Tokyo) 137, 189 (2005). In addition, NF-κB-inducing kinase (NIK),which usually is assigned a role in the alternative activation pathway, has recently been shown to have a role in the activation of IKK. Ramakrishnan et al., Immunity 21, 477 (2004). Thus, there are numerous kinases and other proteins that are associated with the IKK complex that are potential targets for curcumin derivatives.

The use of curcumin derivatives to treat a subject with cancer preferably causes inhibition of AP-1, NF-κB, or both. Inhibition of NF-κB, as defined herein, is a decrease in NF-κB activity. For example, inhibition of NF-κB activity includes a decrease in the activity of NF-κB as a suppressor of apoptosis. Inhibition of NF-κB includes inhibition by direct inhibitors and by indirect inhibitors. Direct inhibition is the direct effect of a curcumin derivative on NF-κB and its activity. For example, one type of direct inhibition of NF-κB is a block of NF-κB DNA interactions. Indirect inhibition, on the other hand, involves the effect of a curcumin derivative on other compounds involved in the regulation of NF-κB that leads to a decrease in NF-κB activity. For example, as phosphorylation of the NF-κB regulator IκB by IκB kinases (IKK) or Src family kinases (SFK) results in a dysregulation of NF-κB, and an according increase in NF-κB activity, inhibition of IKK or SFK by curcumin derivatives provides an example of indirect inhibition.

Inhibition of AP-1, as defined herein, is a decrease in AP-1 activity. For example, inhibition of AP-1 activity includes a decrease in the activity of AP-1 as a suppressor of apoptosis. Inhibition of AP-1 includes inhibition by direct inhibitors and by indirect inhibitors. Direct inhibition is the direct effect of a curcumin derivative on AP-1 (or its subunits) and its activity. Indirect inhibition, on the other hand, involves the effect of a curcumin derivative on other compounds involved in the regulation of AP-1 that leads to a decrease in AP-1 activity. For example, indirect inhibition of AP-1 activity may occur as a result of an affect on AP-1 activating proteins such as mitogen-activated protein kinases (MEPK) or c-Fos-regulating kinase (FRK).

Curcumin derivatives have also been shown to provide antitumor activity through effects on other proteins. For example, curcumin derivatives may affect heat shock protein 90 (HSP90), as described in U.S. Provisional Patent Application No. 60/578,643, entitled “Method and Compounds for Cancer Treatment Utilizing HSP90 as a Direct or Ultimate Target for Small Molecule Inhibitors,” filed Jun. 10, 2004, and U.S. Provisional Patent Application No. 60/736,921, entitled “Method and Compounds for Cancer Treatment Utilizing HSP90 as a Direct or Ultimate Target for Small Molecule Inhibitors,” filed Nov. 15, 2005, both by Vander Jagt et al. and incorporated herein by reference. Curcumin derivatives may also affect glutathione-S-transferase, as described in U.S. Provisional Patent Application No. 60/695,046, entitled “Glutathione S-Transferase Inhibition by Anti-Cancer Curcumin Analogs,” filed Jun. 29, 2005, also by Vander Jagt et al. and incorporated herein by reference.

The cancer treated by the method of the invention may be any of the forms of cancer known to those skilled in the art or described herein. Cancer that manifests as both solid tumors and cancer that instead forms non-solid tumors as typically seen in leukemia can be treated.

The effectiveness of treatment may be measured by evaluating a reduction in tumor load or decrease in tumor growth in a subject in response to the administration of curcumin derivatives. The reduction in tumor load may be represent a direct decrease in mass, or it may be measured in terms of tumor growth delay, which is calculated by subtracting the average time for control tumors to grow over to a certain volume from the time required for treated tumors to grow to the same volume. The subject is preferably a mammal, such as a domesticated farm animal (e.g., cow, horse, pig) or pet (e.g., dog, cat). More preferably, the subject is a human.

Identification of Agents

Another aspect of the invention includes methods for identifying curcumin derivatives that may be used to treat a subject with cancer by inhibiting AP-1 or NF-κB activity. Potential agents suitable for testing are referred to herein as “candidate agents.” The method involves exposing AP-1 or NF-κB to the candidate agent and determining whether or not its activation by an AP-1 or NF-κB activator (respectively) is inhibited. As AP-1 and NF-κB are transcription factors, their activation is most readily evaluated in a cell assay. However, AP-1 or NF-κB activation can also be evaluated in cell-free systems using techniques readily known by those skilled in the art. Sources for candidate agents include, for instance, chemical compound libraries, and extracts of plants and other vegetations.

For example, in one embodiment, the method for identifying a curcumin derivative that may be used to treat a subject with cancer by inhibiting NF-κB involves contacting a cell including an activatable NF-κB with a curcumin derivative, contacting the cell with an NF-κB activator (e.g., TNF-α or IL-1) and determining the extent of the decrease of NF-κB activation by the curcumin derivative. A candidate agent that results in a decrease of NF-κB activation is accordingly identified by this method as a curcumin derivative that may be used to treat a subject with cancer. For example, a cell assay suitable for identifying curcumin derivatives that are useful for treating a subject with cancer is provided by Example 3, herein.

In a further exemplary embodiment, the method for identifying a curcumin derivative that may be used to treat a subject with cancer by inhibiting AP-1 involves contacting a cell including an activatable AP-1 with a curcumin derivative, contacting the cell with an AP-1 activator (e.g., TNF-α or phorbol 12-myristate 13-acetate) and determining the extent of the decrease of AP-1 activation by the curcumin derivative. A candidate agent that results in a decrease of AP-1 activation is accordingly identified by this method as a curcumin derivative that may be used to treat a subject with cancer. For example, a cell assay suitable for identifying curcumin derivatives that are useful for treating a subject with cancer is provided by Example 5, herein.

Candidate agents may also be tested in animal models. Typically, the animal model is one for the study of cancer. The study of various cancers in animal models (for instance, mice) is a commonly accepted practice for the study of human cancers. For instance, the nude mouse model, where human tumor cells are injected into the animal, is commonly accepted as a general model useful for the study of a wide variety of cancers, including prostate cancer (see, for instance, Polin et al., Investig. New Drugs, 15:99-108 (1997)). Results are typically compared between control animals treated with candidate agents and the control littermates that did not receive treatment. Transgenic animal models are also available and are commonly accepted as models for human disease (see, for instance, Greenberg et al., Proc. Natl. Acad. Sci. USA, 92:3439-3443 (1995)). Candidate agents can be used in these animal models to determine if a candidate agent decreases one or more of the symptoms associated with the cancer, including, for instance, cancer metastasis, cancer cell motility, cancer cell invasiveness, and the combination thereof.

Administration and Formulation of Curcumin Derivatives

The present invention provides a method for using a composition that includes one or more small molecule inhibitors of the invention to treat a subject with cancer by administering curcumin derivatives alone, or along with one or more pharmaceutically acceptable carriers. One or more curcumin derivatives with demonstrated biological activity can be administered to a subject in an amount alone or together with other active agents and with a pharmaceutically acceptable buffer. The a composition that includes one or more small molecule inhibitors of the invention can be combined with a variety of physiological acceptable carriers for delivery to a patient including a variety of diluents or excipients known to those of ordinary skill in the art. For example, for parenteral administration, isotonic saline is preferred. For topical administration, a cream, including a carrier such as dimethylsulfoxide (DMSO), or other agents typically found in topical creams that do not block or inhibit activity of the peptide, can be used. Other suitable carriers include, but are not limited to, alcohol, phosphate buffered saline, and other balanced salt solutions.

Methods of administering small molecule therapeutic agents are well-known in the art. Reference is made, for example, to US Pat. Publ. 2001-0051184 A1, published Dec. 13, 2001 (Heng) concerning illustrative modes of administration of curcumin analogs as well as dosage amounts and protocols.

The formulations may be conveniently presented in unit dosage form and may be prepared by any of the methods well known in the art of pharmacy. Preferably, such methods include the step of bringing the active agent into association with a carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The curcumin derivatives can be administered as a single dose or in multiple doses. Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949.

The agents of the present invention are preferably formulated in pharmaceutical compositions and then, in accordance with the methods of the invention, administered to a subject, such as a human patient, in a variety of forms adapted to the chosen route of administration. The formulations include, but are not limited to, those suitable for oral, rectal, vaginal, topical, nasal, ophthalmic, or parental (including subcutaneous, intramuscular, intraperitoneal, intratumoral, and intravenous) administration.

Formulations suitable for parenteral administration conveniently include a sterile aqueous preparation of the active agent, or dispersions of sterile powders of the active agent, which are preferably isotonic with the blood of the recipient. Parenteral administration of curcumin derivatives (e.g., through an I.V. drip) is an additional form of administration. Isotonic agents that can be included in the liquid preparation include sugars, buffers, and sodium chloride. Solutions of the active agent can be prepared in water, optionally mixed with a nontoxic surfactant. Dispersions of the active agent can be prepared in water, ethanol, a polyol (such as glycerol, propylene glycol, liquid polyethylene glycols, and the like), vegetable oils, glycerol esters, and mixtures thereof. The ultimate dosage form is sterile, fluid, and stable under the conditions of manufacture and storage. The necessary fluidity can be achieved, for example, by using liposomes, by employing the appropriate particle size in the case of dispersions, or by using surfactants. Sterilization of a liquid preparation can be achieved by any convenient method that preserves the bioactivity of the active agent, preferably by filter sterilization. Preferred methods for preparing powders include vacuum drying and freeze drying of the sterile injectible solutions. Subsequent microbial contamination can be prevented using various antimicrobial agents, for example, antibacterial, antiviral and antifungal agents including parabens, chlorobutanol, phenol, sorbic acid, thimerosal, and the like. Absorption of the active agents over a prolonged period can be achieved by including agents for delaying, for example, aluminum monostearate and gelatin.

Formulations of the present invention suitable for oral administration may be presented as discrete units such as tablets, troches, capsules, lozenges, wafers, or cachets, each containing a predetermined amount of the active agent as a powder or granules, as liposomes containing the curcumin derivatives, or as a solution or suspension in an aqueous liquor or non-aqueous liquid such as a syrup, an elixir, an emulsion, or a draught. Such compositions and preparations typically contain at least about 0.1 wt-% of the active agent. The amount of curcumin derivatives (i.e., active agent) is such that the dosage level will be effective to produce the desired result in the patient.

Nasal spray formulations include purified aqueous solutions of the active agent with preservative agents and isotonic agents. Such formulations are preferably adjusted to a pH and isotonic state compatible with the nasal mucous membranes. Formulations for rectal or vaginal administration may be presented as a suppository with a suitable carrier such as cocoa butter, or hydrogenated fats or hydrogenated fatty carboxylic acids. Ophthalmic formulations are prepared by a similar method to the nasal spray, except that the pH and isotonic factors are preferably adjusted to match that of the eye. Topical formulations include the active agent dissolved or suspended in one or more media such as mineral oil, petroleum, polyhydroxy alcohols, or other bases used for topical pharmaceutical formulations.

The tablets, troches, pills, capsules, and the like may also contain one or more of the following: a binder such as gum tragacanth, acacia, corn starch or gelatin; an excipient such as dicalcium phosphate; a disintegrating agent such as corn starch, potato starch, alginic acid, and the like; a lubricant such as magnesium stearate; a sweetening agent such as sucrose, fructose, lactose, or aspartame; and a natural or artificial flavoring agent. When the unit dosage form is a capsule, it may further contain a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain one or more of a sweetening agent, a preservative such as methyl- or propylparaben, an agent to retard crystallization of the sugar, an agent to increase the solubility of any other ingredient, such as a polyhydric alcohol, for example glycerol or sorbitol, a dye, and flavoring agent. The material used in preparing any unit dosage form is substantially nontoxic in the amounts employed. The active agent may be incorporated into sustained-release preparations and devices.

The curcumin derivatives of the invention can be incorporated directly into the food of the mammal's diet, as an additive, supplement, or the like. Thus, the invention further provides a food product containing a curcumin derivative of the invention. Any food is suitable for this purpose, although processed foods already in use as sources of nutritional supplementation or fortification, such as breads, cereals, milk, and the like, may be more convenient to use for this purpose.

Small molecule inhibitors such as curcumin derivatives are well-suited for direct or indirect (ultimate) blocking of tumor-associated AP-1 or NF-κB activity, as they are usually easily synthesized and readily taken up by mammalian cells. In some embodiments, the small molecule inhibitor is derivatized or conjugated with a carrier molecule according to methods well known in the art, so as to increase targeting efficiency and/or the rate of cellular uptake, for example by being covalently linked to a ligand that binds to a cell surface receptor.

Preparation of the Compounds

Compounds of the invention may be synthesized by synthetic routes that include processes derivativeous to those well known in the chemical arts, particularly in light of the description contained herein. The starting materials are generally available from commercial sources such as Aldrich Chemicals (Milwaukee, Wis., USA) or are readily prepared using methods well known to those skilled in the art (e.g., prepared by methods generally described in Louis F. Fieser and Mary Fieser, Reagents for Organic Synthesis, v. 1-19, Wiley, New York, (1967-1999 ed.); Alan R. Katritsky, Otto Meth-Cohn, Charles W. Rees, Comprehensive Organic Functional Group Transformations, v 1-6, Pergamon Press, Oxford, England, (1995); Barry M. Trost and Ian Fleming, Comprehensive Organic Synthesis, v. 1-8, Pergamon Press, Oxford, England, (1991); or Beilsteins Handbuch der organischen Chemie, 4, Aufl. Ed. Springer-Verlag, Berlin, Germany, including supplements (also available via the Beilstein online database)).

For illustrative purposes, the reaction schemes depicted below provide potential routes for synthesizing the compounds of the present invention as well as key intermediates. For more detailed description of the individual reaction steps, see the EXAMPLES section below. Generally, compounds of the present invention are prepared by reacting a pair of aryl aldehydes using an aldol reaction. For example, curcumin derivatives including a 7-carbon linker may be prepared by reacting 2,4-pentanedione with a substituted arylaldehyde in an aldol-type reaction according to the procedure described by Pabon (Pabon, H. J. J. Recueil 1964, 83, 379). In a further example, curcumin derivatives including a 5-carbon linker may be prepared by reaction of acetone with substituted arylaldehydes in a base catalyzed aldol reaction, as described by Masuda et al. (Masuda et al., Phytochemistry 1993, 32, 1557), and curcumin derivatives including a 3-carbon linker (also referred to as chalcones) can be prepared by reaction of a substituted arylaldehyde with a substituted aceto-aryl compound (e.g., acetophenone) in a base catalyzed aldol reaction as described by Kohler and Chadwell (Kohler et al., Org. Synth., Coll. 1932, 1, 78).

Those skilled in the art will appreciate that other synthetic routes may be used to synthesize the compounds of the invention. Although specific starting materials and reagents are depicted in the reaction schemes and discussed below, other starting materials and reagents can be easily substituted to provide a variety of derivatives and/or reaction conditions. In addition, many of the compounds prepared by the methods described below can be further modified in light of this disclosure using conventional methods well known to those skilled in the art.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Chemical Synthesis of Curcumin Derivatives

Several derivatives were synthesized that have some structural similarity to curcumin. The following is a discussion of the analogs that were synthesized and the methods used to accomplish the structural changes. Spectral data were useful in characterizing structural changes in the molecules. Proton and carbon nuclear magnetic resonance spectroscopies (NMR) were used to detect functional groups in the curcumin analogs. The following schemes summarize the procedures used to prepare the three series of curcumin analogs. Analogs in series 1, which retain the 7-carbon spacer contained in curcumin, were prepared as shown in Schemes 6-11. Analogs in series 2, which contain a 5-carbon spacer, were prepared as shown in Schemes 12-27. Analogs in series 3, which contain a 3-carbon spacer, were prepared as shown in Schemes 28-37.

Synthesis of 7-Carbon Spacer Analogs

Analogs 3a-3i, contain two aryl rings separated by an unsaturated 7-carbon spacer having two carbonyls (Schemes 1 and 2). The aryl rings contain different substituents in various positions on the ring. These analogs were designed to test the importance of the type of substituent and its location on the aryl ring. Analogs 3a-3h, as shown in Scheme 6, were prepared following the procedure described by Pabon (Pabon, Recueil, 1964, 83, 379-386). 2,4-Pentanedione (2) was reacted with boric anhydride to give the boron/pentanedione complex. The complex was then reacted with the appropriately substituted benzaldehyde (1a-1h), tributyl borate, and butylamine in dry ethyl acetate in an aldol type reaction followed by hydrolysis with warm dilute hydrochloric acid to give curcumin (3a) or one of its analogs 3b-3h. The formation of the products was verified by proton NMR by the appearance of a pair of doublets in the aromatic region with J values of 15.5-16.5 Hz for the alkene protons present in the spacer. Also observed in the proton NMR was the loss of a signal at ˜10 ppm for the aldehyde proton in the starting benzaldehyde and the loss of signals at 1.89 ppm and 2.08 ppm for the methyl protons on 2,4-pentanedione (2). The structures were also verified by carbon NMR by the appearance of a signal at ˜182 ppm for the keto-enol carbonyl carbon and the loss of a signal at ˜195 ppm for the aldehyde carbon in the starting benzaldehyde (1a-1h). Also absent from the carbon NMR were signals at 24.1 ppm and 30.2 ppm for the methyl carbons on 2,4-pentanedione (2). 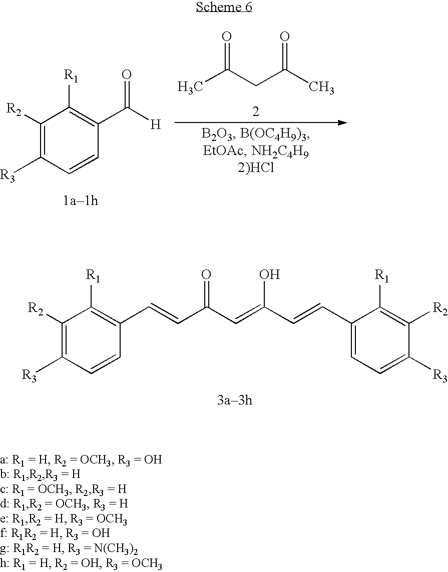
10 Analog 3d, which is not in the literature, was verified by elemental analysis.

Scheme 7 describes the synthesis of analog 3i. Analog 3i was also prepared following the procedure described by Pabon (Pabon, Recueil, 1964, 83, 379-386). 2,4-Pentanedione (2) was reacted with boric anhydride in dry ethyl acetate at 40° C. to give the boron/pentanedione complex. The complex was then reacted with 3,4-dimethoxybenzaldehyde (1i), tributyl borate, and butylamine in dry ethyl acetate at 40° C. in an aldol type reaction followed by hydrolysis with warm dilute hydrochloric acid to give analog 3i. The formation of the product was verified by proton NMR by the appearance of a pair of doublets in the aromatic region with J values of 15.9 Hz for the alkene protons present in the spacer. Also observed in the proton NMR was the loss of a signal at 9.85 ppm for the aldehyde proton in the starting benzaldehyde (1i) and the loss of signals at 1.89 ppm and 2.08 ppm for the methyl protons on 2,4-pentanedione (2). The structure was also verified by carbon NMR by the appearance of a signal at 183.0 ppm for the keto-enol carbonyl carbon and the loss of a signal at 190.9 ppm for the aldehyde carbon in the starting benzaldehyde (1i). Also absent from the carbon NMR were signals at 24.1 ppm and 30.2 ppm for the methyl carbons on 2,4-pentanedione (2).

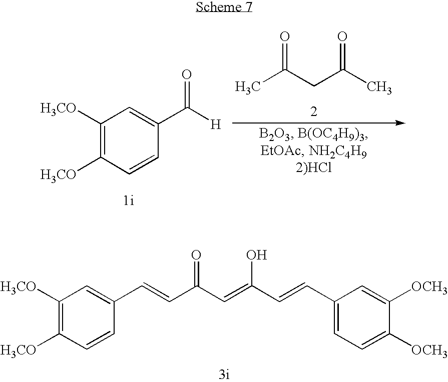

Two additional curcumin analogs, 6a and 6b, were prepared as shown in Scheme 8. Analogs 6a and 6b contain two aryl rings separated by an unsaturated 7-carbon spacer having two carbonyls and a single methyl substituent attached to the central methylene carbon. These analogs were designed to test the importance of a methyl substituent on the central methylene carbon. 3-Methyl-2,4-pentanedione (5) was first synthesized by reaction of 2,4-pentanedione (2) with potassium carbonate and methyl iodide (4) in acetone at 56° C. in a substitution reaction following the procedure described by Markham and Price (Markham et al., Org. Synth. Coll. Vol. V. 785-790). This reaction gave the monomethyl substituted product as the major product along with small amounts of both unreacted 2,4-pentanedione (2) and of the dimethyl substituted product. The formation of the product was verified by proton NMR by the appearance of a doublet at 1.12 ppm for the methyl protons and a quartet at 3.52 ppm for the remaining methylene proton. Analogs 6a and 6b were then prepared from compound 5, following the procedure described by Pabon (Pabon, Recueil, 1964, 83, 379-386), by reaction with boric anhydride under a nitrogen atmosphere to give the boron/pentanedione complex. The complex was then reacted with 4-hydroxy-3-methoxybenzaldehyde (1a) or benzaldehyde (1b), tributyl borate, and butylamine in an aldol type reaction followed by hydrolysis with warm dilute hydrochloric acid to give 6a and 6b respectively. The formation of products was verified by proton NMR by the appearance of a pair of doublets in the aromatic region with J values of 15.5 Hz for the alkene protons in the spacer and the loss of signals at 1.92 ppm and 2.00 ppm for the terminal methyl protons on 3-methyl-2,4-pentanedione (5). The structures were also verified by carbon NMR by the appearance of a signal at ˜182.2 ppm for the keto-enol carbonyl carbon. The carbon NMR also showed the loss of signals at 23.0 ppm and 28.4 ppm for the terminal methyl carbons on 3-methyl-2,4-pentanedione (5). The carbon NMR of analog 6b also showed the loss of a signal at 192.1 ppm for the aldehyde carbon in the starting benzaldehyde (1b), whereas in analog 6a, a signal is present at 196.0 ppm due to the carbonyl carbon of the diketo form and not the aldehyde carbon of the starting benzaldehyde (1b). Both analogs 6a and 6b, which are not in the literature, were verified by elemental analysis.

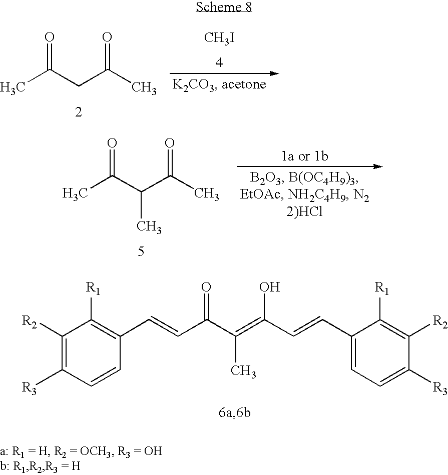

Two additional curcumin analogs, 9a and 9b, were prepared as shown in Scheme 9. Analogs 9a and 9b contain two aryl rings separated by an unsaturated 7-carbon spacer having two carbonyls and a single benzyl substituent attached to the central methylene carbon. These analogs were designed to test the importance of a benzyl substituent on the central methylene carbon. The starting material 3-benzylidene-2,4-pentanedione (7), was prepared by a Knoevenagel condensation reaction of 2,4-pentanedione (2) with benzaldehyde (1b), glacial acetic acid and piperdine in benzene at 65° C. following the procedure described by Antonioletti (Antonioletti et al., Tetrahedron 2002, 58(3), 589-596). The formation of the product was verified by proton NMR by the appearance of a signal at 7.45 ppm for the alkene proton and the loss of a signal at 5.37 ppm for the central methylene proton on compound 2. 3-Benzyl-2,4-pentanedione (8) was prepared by reaction of compound 7 with palladium on activated carbon under a hydrogen atmosphere on a Parr apparatus in a reduction reaction following the procedure described by Venkateswarlu (Venkateswarlu et al., Asian J. Chem. 2000, 12(1), 141-144). The formation of the product was verified by proton NMR by the appearance of triplet at 4.01 ppm for the central methylene proton and a doublet at 3.11 ppm for the benzylic protons of the diketo form of compound 8. Also observed in the proton NMR is a singlet at 3.62 ppm for the benzylic protons of the keto-enol form of compound 8. The proton NMR also shows the loss of a signal at 7.45 ppm for the alkene proton in compound 7. Analogs 9a and 9b were then prepared from compound 8, following the procedure described by Pabon (Pabon, Recueil, 1964, 83, 379-386), by reaction with boric anhydride under a nitrogen atmosphere to give the boron/pentanedione complex. The complex was then reacted with 4-hydroxy-3-methoxybenzaldehyde (1a) or benzaldehyde (1b), tributyl borate, and butylamine in an aldol type reaction followed by hydrolysis with warm dilute hydrochloric acid to give 9a and 9b respectively. The formation of the products was verified by proton NMR by the appearance of a pair of doublets in the aromatic region with J values of 15.1-15.6 Hz for the alkene protons in the spacer and the loss of signals at ˜2.05 ppm for the methyl 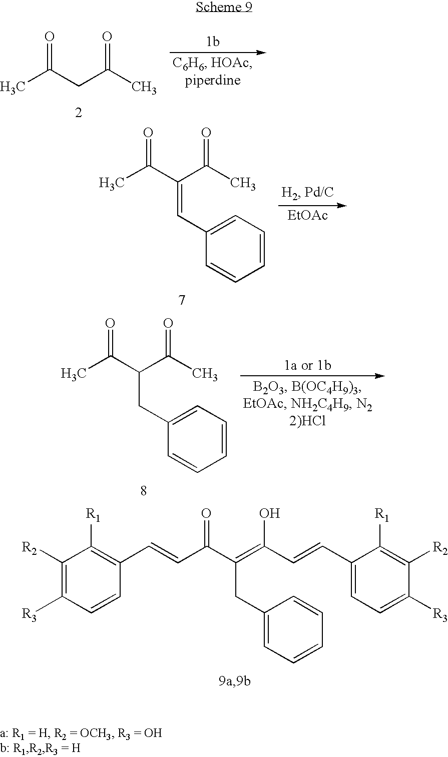
protons of 3-benzyl-2,4-pentanedione (8). The structures were also verified by carbon NMR by the appearance of a signal at ˜183.3 ppm for the keto-enol carbonyl carbon. Also observed in the carbon NMR was the loss of signals at 22.9 ppm and 29.4 ppm for the methyl carbons of 3-benzyl-2,4-pentanedione (8). The carbon NMR of analog 9b also showed the loss of a signal at 192.1 ppm for the aldehyde carbon in the starting benzaldehyde (1b); whereas in analog 9a, a signal was present at 194.0 ppm for the carbonyl carbon of the diketo form of the analog. Analog 9a, which is not in the literature, was verified by elemental analysis.

Two additional curcumin analogs, 11b and 12b, were prepared as shown in Scheme 10. Analogs 11b and 12b contain two aryl rings separated by an unsaturated 7-carbon spacer having two carbonyls. Analog 11b contains two methyl substituents attached to the central methylene carbon, whereas analog 12b contains two benzyl substituents attached to the central methylene carbon. These analogs were designed to test the importance of two substituents on the central methylene carbon. Analogs 11b and 12b were prepared by reaction of analog 3b with sodium hydroxide, tetrabutylammonium chloride and either methyl iodide (4) or benzyl bromide (10) in dichloromethane at 40° C. in a substitution reaction following the procedure described by Pedersen (Pedersen et al., Liebigs Ann. Chem. 1985, 8, 1557-1569). The formation of the products was verified by proton NMR by the appearance of a signal at 1.48 ppm for the methyl protons in analog 11b and a signal at 3.39 ppm for the benzylic protons in analog 12b. Also observed in the proton NMR was the loss of a signal at 5.84 ppm for the central methylene proton in analog 3b. Pedersen (Pedersen et al., Liebigs Ann. Chem. 1985, 8, 1557-1569) observed the monosubstituted product, analog 9b. However, we observed only the disubstituted product, analog 12b. To verify the formation of analogs 11b and 12b, integration of the proton NMR was examined. The signal for the benzylic protons at 3.39 ppm was integrated and compared to each of the alkene signals in the aromatic region. The benzylic singlet at 3.39 ppm in analog 12b integrates for four protons and the two alkene signals in the aromatic region integrate for four protons which is to be expected if the product is disubstituted. The same observation was made in analog 11b. The methyl singlet at 1.48 ppm integrates for six protons compared to four protons for the alkene signals indicating the presence of the disubstituted product. The structures were also verified by carbon NMR by the shift of the methylene signal from ˜101.6 ppm to ˜66 ppm and the appearance of signals at 21.1 ppm (11b) for the methyl carbons and 37.0 ppm (12b) for the benzylic carbons. Analog 11b, which is not in the literature, was verified by elemental analysis.

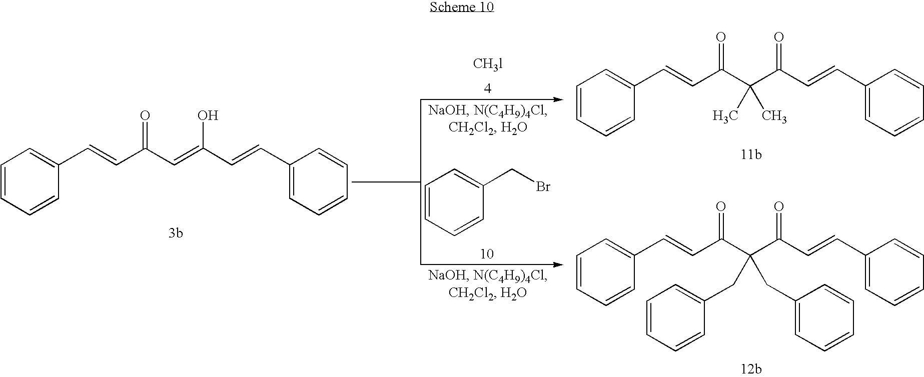

Eight additional curcumin analogs, 13a, 13b, 14a, 14b, 15a, 15b, 16b, and 17b, were prepared as shown in Scheme 11. These analogs contain two identical aryl rings separated by a saturated 7-carbon spacer containing two carbonyls. The analogs were designed to test the importance of saturation in the spacer. Analogs 13a, 13b, 14a, 14b, 15a, 15b, 16b, and 17b were prepared from analogs 3a, 3b, 6a, 6b, 9a, 9b, 11b, and 12b respectively by reduction with palladium on activated carbon under a hydrogen atmosphere on a Parr apparatus following the procedure described by Venkateswarlu (Venkateswarlu et al., Asian J. Chem. 2000, 12(1), 141-144). The formation of the products was verified by proton NMR by the appearance of two multiplets at ˜2.75 ppm for the alkane protons in the spacer. Also observed in the proton NMR was the loss of two doublets in the aromatic region for the alkene protons. The structures were also verified by carbon NMR by the appearance of signals at ˜30.5 ppm and ˜42.5 ppm for the alkane carbons. The carbon NMR also showed the loss of two signals in the aromatic region for the alkene carbons. Analogs 14a, 14b, 15a, 15b, and 17b, which are not in the literature, were verified by high resolution mass spectroscopy. Analog 16b, which is not in the literature, was verified by elemental analysis. 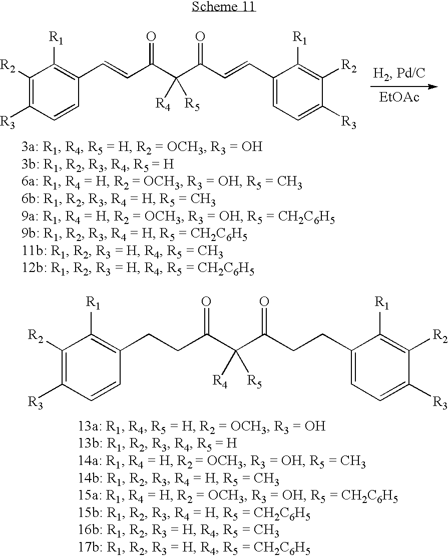
Synthesis of 5-Carbon Spacer Analogs

Analogs in series 2, which contain a shorter 5-carbon spacer than in curcumin, were prepared as shown in Schemes 12-27. Analogs 20a-20g, 20i, 20k-20ac and 20ae-20ah, as shown in Schemes 12-16, all contain two identical aryl rings separated by an unsaturated 5-carbon spacer having a single carbonyl. These analogs were designed to test the importance of the length of the spacer and the type of functional group and location of the substituent on the aryl ring. Analog 20a, which contains the same aryl substituents as curcumin was prepared as shown in Scheme 12 following the procedure as described by Masuda (Masuda et al., Phytochemistr 1993, 32(6), 1557-1560). 4-Methoxymethyloxy-3-methoxybenzaldehyde (1j) was prepared by reaction of 4-hydroxy-3-methoxybenzaldehyde (1a) with potassium carbonate and chloromethyl methyl ether (18) in a substitution reaction to protect the phenol. Protection was necessary because the aldol reaction on the phenol did not proceed, even upon heating to reflux. The formation of compound 1j was verified by proton NMR by the appearance of signals at 5.21 ppm for the methylene protons and 3.40 ppm for the methyl protons of the protecting group. 1,5-Bis(4-methoxymethyloxy-3-methoxyphenyl)-1,4-pentadien-3-one (20j) was prepared by reaction of compound 1j with acetone (19) and sodium hydroxide in an aldol reaction. The formation of the product was verified by proton NMR by the appearance of a pair of doublets at 7.69 ppm and 6.97 ppm with J values of 15.9 Hz for the alkene protons in the spacer. The final step in the preparation of analog 20a was the removal of the groups protecting the phenols. The removal of the protecting groups was accomplished by reaction of compound 20j with a catalytic amount of concentrated hydrochloric acid in methanol at 65° C. to give the phenol, analog 20a. The formation of 20a was verified by proton NMR by the loss of signals at 3.40 ppm and 5.21 ppm for the protons of the protecting group on compound 20j. The structure was also verified by carbon NMR by the loss of signals at 56.4 ppm and 95.2 ppm for the carbons of the protecting groups on compound 20j.

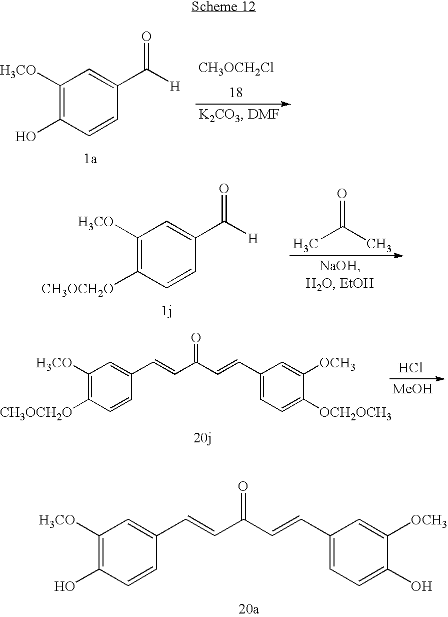

Scheme 13 describes the synthesis of analogs 20b-20g, 20i, and 20k-20ac. Analogs 20b-20g, 20i, and 20k-20ac were prepared following the procedure described by Masuda (Masuda et al., Phytochemistry 1993, 32(6), 1557-1560). A substituted benzaldehyde (1b-1g, 1i, and 1k-1ac) was reacted with acetone (19) and sodium hydroxide in an aldol reaction to give analogs 20b-20g, 20i, and 20k-20ac. The formation of the products was verified by proton NMR by the appearance of a pair of doublets in the aromatic region with J values between 15.6-16.1 Hz for the alkene protons present in the spacer. Also observed in the proton NMR was the absence of a signal at ˜10 ppm for the aldehyde proton of the starting benzaldehyde (1b-1g, 1i, and 1k-1ac) and a signal at 2.04 ppm for the methyl protons of acetone (19). The structures were also verified by carbon NMR by the appearance of two signals in the aromatic region for the alkene carbons in the spacer. Absent from the carbon NMR was a signal at 30.6 ppm for methyl carbons in acetone (19). Analogs 20s and 20v, which are not in the literature, were verified by elemental analysis.

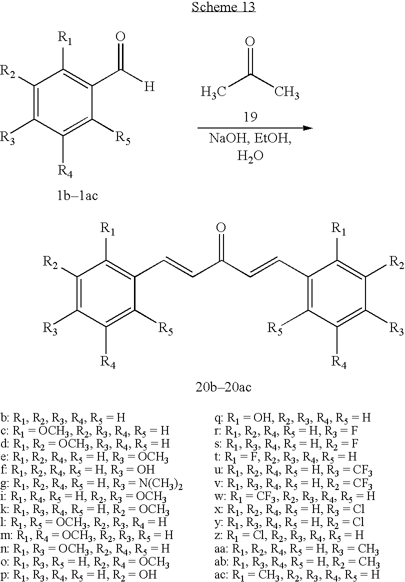

Scheme 14 describes the synthesis of analog 20ae. Analog 20ae was prepared following the procedure described by White and Zoeller (White et al., U.S. Pat. No. 5,395,692 (1995); Chem. Abstr., 123, P84361 n (1995)). 4-Formylbenzoic acid (1ad) was reacted with methanol and thionyl chloride in an esterification reaction to give compound 1ae. The formation of the product was verified by proton NMR by the appearance of a signal at 3.87 ppm for the methyl ester protons. Compound 1ae was then reacted with acetone (19) and sodium hydroxide in an aldol reaction to give analog 20ae. The formation of the product was verified by proton NMR by the appearance of a pair of doublets in the aromatic region with J values of 15.9 Hz and 16.1 Hz for the alkene protons present in the spacer. Also observed in the proton NMR was the loss of a signal at 10.12 ppm for the aldehyde 15 proton of the starting benzaldehyde (1ae) and a signal at 2.04 ppm for the methyl protons of acetone (19). The structure was also verified by carbon NMR by the appearance of two signals in the aromatic region for the alkene carbons. The carbon NMR also showed the loss of a signal at 30.6 ppm for the loss of the methyl carbons of acetone (19).

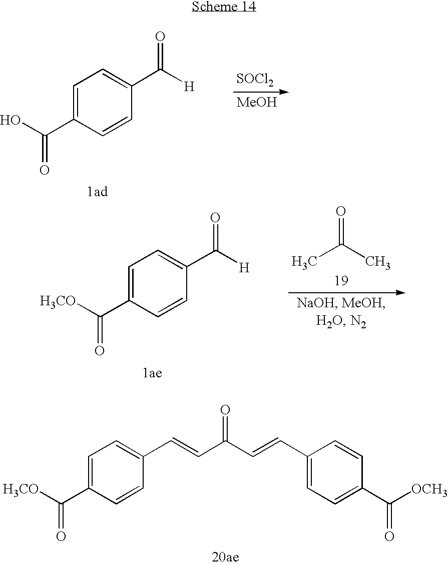

Scheme 15 describes the synthesis of analog 20af. Analog 20af was prepared following the procedure described by Royer (Royer et al., J. Med. Chem. 1995, 38(13), 2427-2432). Analog 20i was demethylated with boron tribromide to give analog 20af. The same reaction was also attempted on analogs 20d and 20l-20o with the anticipation of forming the corresponding tetrahydroxy analogs, however pure stable products could not be obtained. Immediately following chromatography there was a single spot on tic, indicating pure product, however after approximately 24 hours, tic showed a large spot at the origin. In order to verify these results, the tetramethoxymethyl ether analogs of 20d and 20l were deprotected using methods described in Scheme 12 (Masuda et al., Phytochemistry 1993, 32(6), 1557-1560) to give the corresponding tetrahydroxy analogs. The same results were obtained, thus confirming the analogs were decomposing. Analog 20af appeared to be stable and was tested immediately. The formation of analog 20af was verified by proton NMR by the appearance of signals at 9.63 ppm and 9.15 ppm for the phenolic protons. Also observed in the proton NMR was the loss of signals at 3.94 ppm and 3.92 ppm for the methyl protons on analog 20i. The structure was also verified by carbon NMR by the loss of a signal at 55.9 ppm for the methyl carbons on analog 20i.

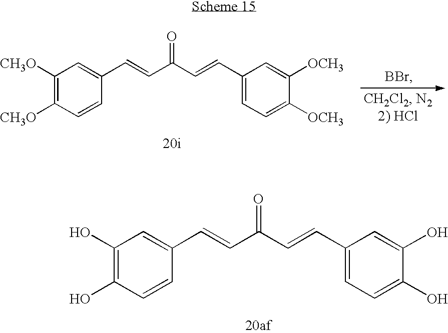

Scheme 16 describes the synthesis of analogs 20ag and 20ah. Analogs 20ag and 20ah were prepared following the procedure described by Suarez (Suarez et al., World Patent 2004,047,716 (2004); Chem. Abstr., 141, 38433 (2004)). Analog 20a or 20f was reacted with acetic anhydride in the presence of pyridine in an esterification reaction to give analogs 20ag and 20ah respectively. The formation of the products was verified by proton NMR by the appearence of a signal at 2.31 ppm for the methyl protons of the acetyl groups. The structures were also verified by carbon NMR by the appearance of a signal at ˜20.9 ppm for the methyl carbons of the acetyl groups and a signal at ˜168.7 ppm for the carbonyls of the acetyl groups. Analog 20ag and 20ah, which are not in the literature, were verified by high resolution mass spectroscopy.

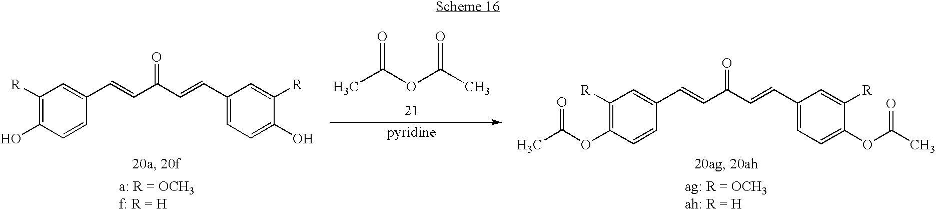

Two additional 5-carbon spacer analogs, 23 and 25, were prepared as shown in Scheme 17. Analogs 23 and 25 contain two naphthalene rings separated by an unsaturated 5-carbon spacer having a single carbonyl. These analogs were designed to test the importance of naphthalene rings. Compound 22 or 24 was reacted with acetone (19) and sodium hydroxide in an aldol reaction following the procedure described by Masuda (Masuda et al., Phytochemistry 1993, 32(6), 1557-1560) to give analogs 23 and 25. The formation of the products was verified by proton NMR by the appearance of a pair of doublets in the aromatic region with J values between 15.7-15.9 Hz for the alkene protons present in the spacer. Also observed in the proton NMR was the loss of a signal at ˜10.25 ppm for the aldehyde proton of the starting naphthaldehydes (22 and 24) and a signal at 2.04 ppm for the methyl protons of 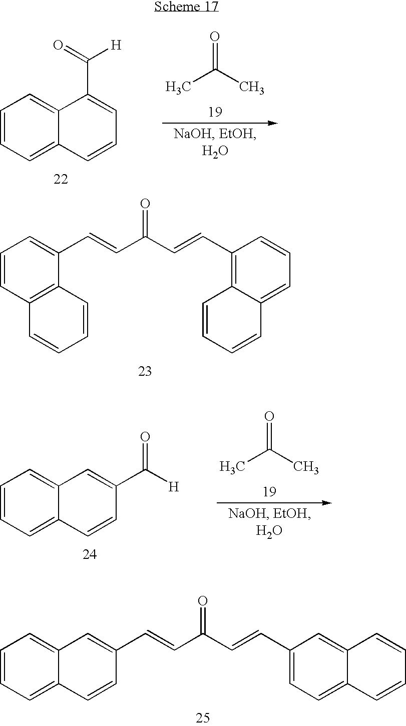
acetone (19). The structures were also verified by carbon NMR by the appearance of two signals in the aromatic region for the alkene carbons on the spacer. The carbon NMR also showed the loss of a signal at 30.6 ppm for the methyl carbons in acetone (19).

Four additional 5-carbon spacer analogs, 28, 29, 31 and 32, were prepared as shown in Scheme 18. Analogs 28, 29, 31 and 32 contain two nitrogen containing aryl rings separated by an unsaturated 5-carbon spacer having a single carbonyl. These analogs were designed to test the importance of nitrogen containing aryl rings. Analogs 28 and 31 were prepared following the procedure described by Zelle and Su (Zelle et al., World Patent 9,820,891 (1998); Chem. Abstr., 129, P23452v (1998)). 4-Pyridinecarboxaldehyde (26) or 3-pyridinecarboxaldehyde (30) was reacted with 1,3-acetonedicarboxylic acid (27) in an aldol type reaction followed by addition of concentrated hydrochloric acid to give analogs 28 and 31 as hydrochloride salts. Analogs 29 and 32, the free bases, were then prepared by shaking analogs 28 and 31 respectively in sodium hydroxide. The formation of analogs 28, 29, 31 and 32 were verified by proton NMR by the appearance of a pair of doublets in the aromatic region with J values between 15.9-16.3 Hz for the alkene protons present in the spacer. Also observed in the proton NMR was the loss of a signal at ˜10.11 ppm for the aldehyde proton in the starting pyridinecarboxaldehydes (26 and 30) and a signal at 3.55 ppm for the methylene protons of 1,3-acetonedicarboxylic acid (27). The structures were also verified by carbon NMR by the appearance of two signals in the aromatic region for the alkene carbons and the loss of a signal at ˜191.3 ppm for the aldehyde carbon of the starting pyridinecarboxaldehyde (26 and 30). Also observed in the carbon NMR was the loss of a signal at 170.3 ppm for the two carboxylic acid carbons and a signal at 50.1 ppm for the methylene carbons in 1,3-acetonedicarboxylic acid (27). The NMR spectra for the uncharged analogs, 29 and 32 were taken in CDCl₃, whereas the charged analogs 28 and 31 were taken in D₂O.

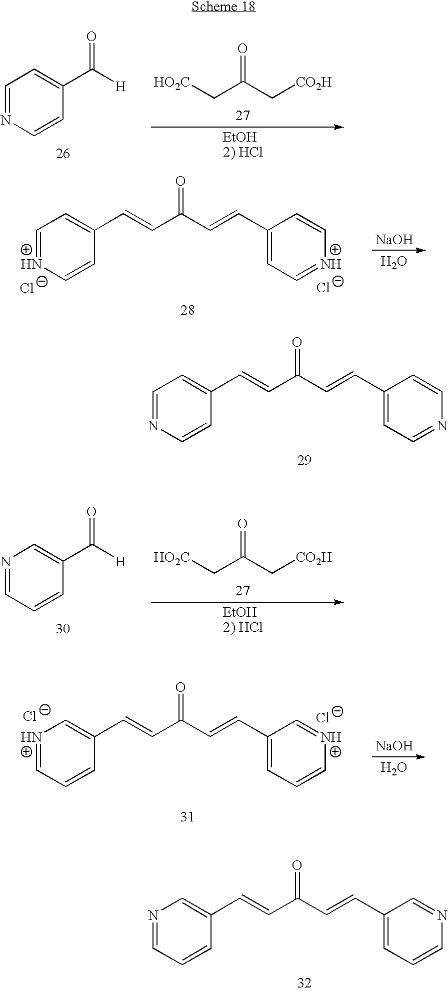

An additional 5-carbon spacer analog, 34, was prepared as shown in Scheme 19. Analog 34 contains two sulfur containing aryl rings separated by an unsaturated 5-carbon spacer having a single carbonyl. This analog was designed to test the importance of thiophene rings. 2-Thiophenecarboxaldehyde (33) was reacted with acetone (19) and sodium hydroxide in an aldol reaction to give analog 34 following the procedure described by Masuda (Masuda et al., Phytochemistry 1993, 32(6), 1557-1560). The formation of analog 34 was verified by proton NMR by the appearance of a pair of doublets in the aromatic region with J values of 15.5 Hz for the alkene protons present in the spacer. Also observed in the proton NMR was the loss of a signal at 9.79 ppm for the aldehyde proton in the starting 2-thiophenecarboxaldehyde (33) and a signal at 2.04 ppm for the methyl protons in acetone (19). The structure was also verified by carbon NMR by the appearance of two signals in the aromatic region for the alkene carbons in the spacer. The carbon NMR also showed 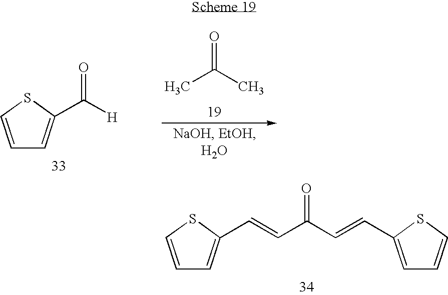
the loss of a signal at 30.6 ppm for the loss of the methyl carbons in acetone (19).

Three additional analogs, 35a, 35e and 35q, were prepared as shown in Schemes 20 and 21. These analogs contain a single aryl ring with an unsaturated 4-carbon tether and a single carbonyl and were designed to test the necessity of two aryl rings. Analog 35a was prepared as shown in Scheme 20 following the procedure described by Masuda (Masuda et al., Phytochemistry 1993, 32(6), 1557-1560). Compound 1j, prepared as previously reported in Scheme 12, was reacted with excess acetone (19) and sodium hydroxide in an aldol reaction to give compound 35j. Protection was necessary because the aldol reaction on the phenol did not proceed, even upon heating to reflux. The formation of compound 35j was verified by proton NMR by the appearance of a pair of doublets in the aromatic region with J values of 16.1 Hz for the alkene protons present on the tether and a signal at 2.34 ppm for the methyl protons present on the tether. Also observed in the proton NMR was the loss of a signal at 9.75 ppm for the aldehyde proton in the starting benzaldehyde (1j). Compound 35j was then reacted with a catalytic amount of concentrated hydrochloric acid to give the phenol, analog 35a. The formation of analog 35a was verified by proton NMR by the loss of signals at 3.48 ppm and 5.24 ppm for the protons of the protecting group in compound 35j. The structure was also verified by carbon NMR by the loss of signals at 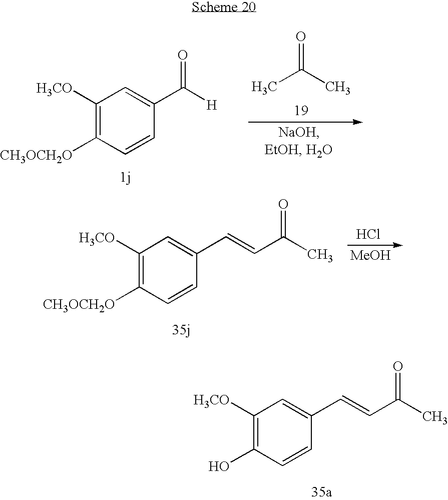
56.2 ppm and 94.8 ppm for the carbons of the protecting group in compound 35j.

Scheme 21 describes the synthesis of analogs 35e and 35q following the procedure described by Masuda (Masuda et al., Phytochemistry 1993, 32(6), 1557-1560). Compound 1e or 1q was reacted with excess acetone (19) and sodium hydroxide in an aldol reaction to give analogs 35e and 35q. The formation of the products was verified by proton NMR by the appearance of a pair of doublets in the aromatic region with J values of 16.3-16.5 Hz for the alkene protons present on the tether and the appearance of a signal at ˜2.37 ppm for the methyl protons on the tether. Also observed in the proton NMR was the loss of a signal at ˜9.94 ppm for the aldehyde proton in the starting benzaldehyde (1e or 1q) and the loss of a signal at 2.04 ppm for the methyl protons of acetone (19). The structures were also verified by carbon NMR by the appearance of two signals in the aromatic region for the alkene carbons present on the tether.

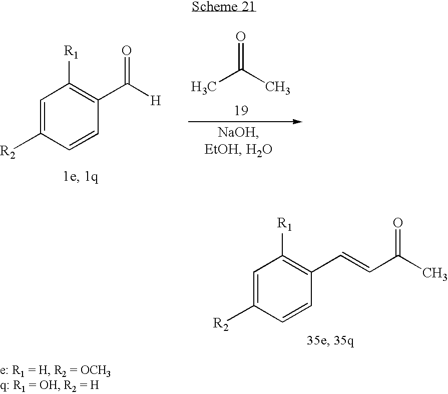

Two additional 5-carbon spacer analogs, 36a and 36e, were prepared as shown in Schemes 22 and 23. These analogs contain two different aryl rings separated by a 5-carbon unsaturated spacer containing a single carbonyl and were designed to test the importance of symmetry in analogs with a 5-carbon spacer. Analog 36a was prepared as shown in Scheme 22 following the procedure described by Masuda (Masuda et al., Phytochemistry 1993, 32(6), 1557-1560). Compound 35j, prepared as shown in Scheme 21, was reacted with benzaldehyde (1b) in an aldol reaction to give compound 36j. The formation of compound 36j was verified by proton NMR by the appearance of a second pair of doublets in the aromatic region for the new alkene in the spacer and the loss of a signal at 2.34 ppm for the methyl protons on the tether in compound 35j. Compound 36j was then reacted with a catalytic amount of concentrated hydrochloric acid to give the phenol, analog 36a. The formation of analog 36a was verified by proton NMR by the loss of signals at 3.5 ppm and 5.2 ppm for the protons of the protecting group in compound 36j. The structure was also verified by carbon NMR by the loss of signals at 56.2 ppm and 94.8 ppm for the carbons of the protecting group in compound 36j.

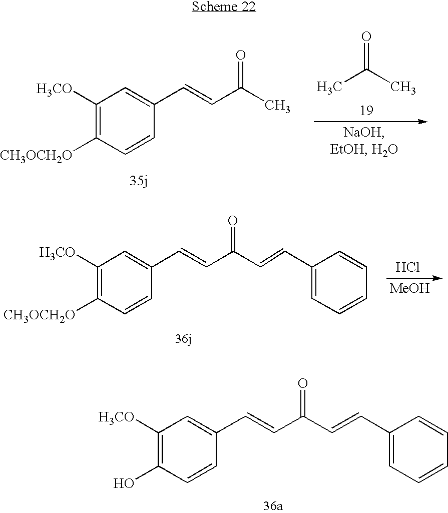

Scheme 23 describes the synthesis of analog 36e. Analog 35e, prepared as shown in Scheme 21, was reacted with benzaldehyde (1b) and sodium hydroxide in an aldol reaction following the procedure described by Masuda (Masuda et al., Phytochemistry 1993, 32(6), 1557-1560) to give analog 36e. The formation of the product was verified by proton NMR by the appearance of a second pair of doublets in the aromatic region with J values of 15.9-16.1 Hz for the new alkene protons present in the spacer and the loss of a signal at 2.34 ppm for the methyl protons on the tether in analog 35e. The structure was also verified by carbon NMR by the appearance of two signals in the aromatic region for the new alkene carbons and the loss of a signal at 27.5 ppm for the methyl carbon on the tether in analog 35e.

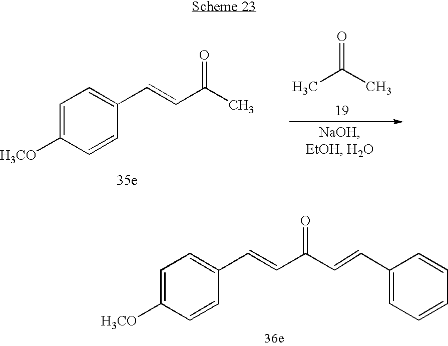

Two additional 5-carbon spacer analogs, 38a and 38b, were prepared as shown in Schemes 24 and 25. These analogs contain two identical aryl rings separated by an unsaturated 5-carbon spacer having a single carbonyl and a saturated ring. Analogs 38a and 38b were designed to test the importance of a ring in the spacer. Analog 38a was prepared as shown in Scheme 24 following the procedure described by Masuda (Masuda et al., Phytochemistry 1993, 32(6), 1557-1560). Compound 1j, prepared as shown in Scheme 12, was reacted with cyclohexanone (37) and sodium hydroxide in an aldol reaction to give compound 38j. The formation of compound 38j was verified by proton NMR by the appearance of a signal at 7.74 ppm for the alkene protons on the spacer and the loss of a signal at 9.75 ppm for the aldehyde proton in the starting benzaldehyde (1j). Compound 38j was then reacted with a catalytic amount of concentrated hydrochloric acid to give the phenol, analog 38a. The formation of the product was verified by proton NMR by appearance of a signal at 5.88 ppm for the phenolic protons and the loss of signals at 3.52 ppm and 5.26 ppm for the 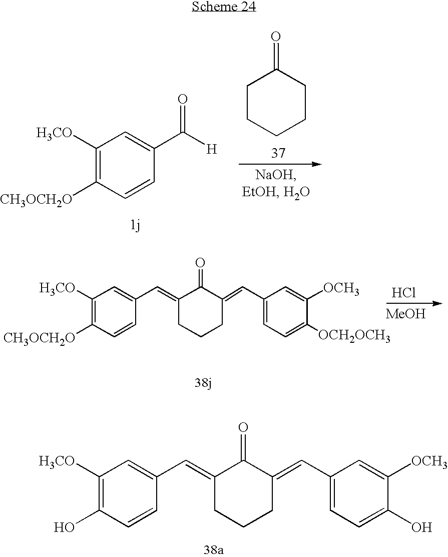
protons of the protecting group in compound 38j. The structure was also verified by carbon NMR by the loss of signals at 55.8 ppm and 95.1 ppm for the carbons of the protecting group in compound 38j.

Scheme 25 describes the synthesis of analog 38b following the procedure described by Masuda (Masuda et al., Phytochemistry 1993, 32(6), 1557-1560). Benzaldehyde (1b) was reacted with cyclohexanone (37) and sodium hydroxide in an aldol reaction to give analog 38b. The formation of the product was verified by proton NMR by the appearance of a signal at 7.80 ppm for the alkene protons on the spacer and the loss of a signal at 9.94 ppm for the aldehyde proton on the starting benzaldehyde (1b). The structure of the product was also verified by carbon NMR by the appearance of two signals in the aromatic region for the alkene carbons on the spacer.

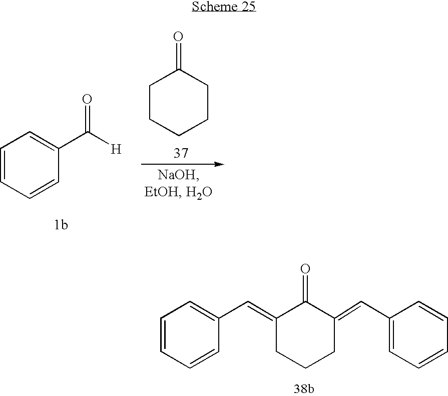

Two additional 5-carbon spacer analogs, 39b and 40b, were prepared as shown in Scheme 26 following the procedure described by Venkateswarlu (Venkateswarlu et al., Asian J. Chem. 2000, 12(1), 141-144). Analog 39b contains two identical aryl rings separated by a saturated 5-carbon spacer and was designed to test the importance of unsaturation in the spacer of series 2 analogs. Analog 40b was designed to test the importance of a carbonyl in the spacer. Analogs 39b and 40b were prepared by reduction of analog 20b with palladium on activated carbon under a hydrogen atmosphere on a Parr apparatus. A mixture containing analogs 39b and 40b was obtained and separated by chromatography. The formation of analog 39b was verified by proton NMR by the appearance of triplets at 2.76 ppm and 2.97 ppm for the methylene protons on the spacer. The proton NMR also showed the loss of a pair of doublets in the aromatic region for the alkene protons. The structure was also verified by carbon NMR by the appearance of signals at 29.6 ppm and 44.2 ppm for the methylene carbons on the spacer and the loss of two signals in the aromatic region for the alkene carbons on the spacer in analog 20b. The formation of analog 40b was verified by proton NMR by the appearance of a pentet for the proton on the carbon bearing the hydroxyl group and multiplets at 1.85 ppm and 2.77 ppm for the methylene protons on the spacer. The structure was also verified by carbon NMR by the appearance of signals at 32.1 ppm, 39.2 ppm and 70.8 ppm for the carbon bearing the hydroxyl group and for the methylene carbons on the spacer. The carbon NMR also shows the loss of two signals in the aromatic region for the alkene carbons on the spacer and the loss of a signal at 188.7 ppm for the carbonyl carbon in analog 20b.

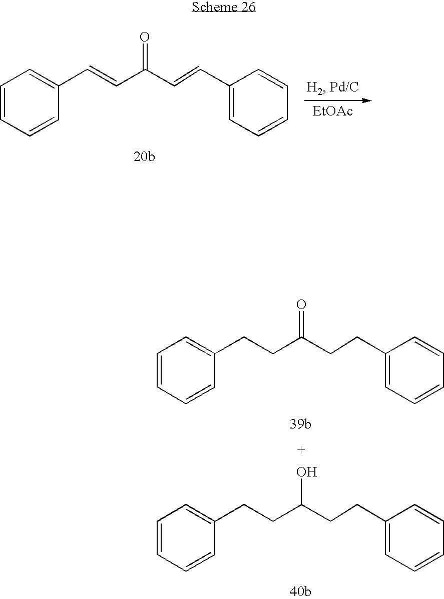

Two additional 5-carbon spacer analogs, 42b and 43b, were prepared as shown in Scheme 27 following the procedure described by Yadav and Kapoor (Yadav et al., Tetrahedron 1996, 52(10), 3659-3668). These analogs contain two identical aryl rings separated by a saturated 5-carbon spacer containing both a carbonyl and two epoxide rings. These analogs were designed to test the importance of epoxide rings on the spacer. Analogs 42b and 43b were prepared by reaction of analog 20b with t-butyl hydroperoxide and aluminum oxide-potassium fluoride in an epoxidation reaction. A mixture containing analog 42b and analog 43b was formed and the trans/trans isomer, analog 42b, was separated from the cis/cis isomer, analog 43b, through recrystallization from ethanol as described by Yadav and Kapoor (Yadav et al., Tetrahedron 1996, 52(10), 3659-3668). The formation of the products was verified by proton NMR by the appearance of a pair of doublets at 3.30 ppm and 4.09 ppm for the alkane protons on the spacer in analog 42b. Analog 43b has a pair of doublets at 3.72 ppm and 4.18 ppm for the alkane protons on the spacer. Also observed in the proton NMR was the loss of two signals in the aromatic region for the alkene protons on the spacer in analog 20b. The structures were also verified by carbon NMR by the appearance of a two signals at ˜59.9 ppm for the alkane carbons on the spacer. The carbon NMR also showed the loss of two signals in the aromatic region for the alkene carbons on the spacer in analog 20b. 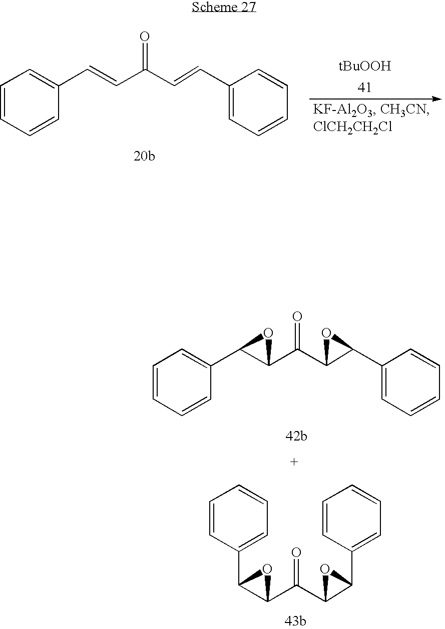
Synthesis of 3-Carbon Spacer Analogs

Analogs in series 3, which contain a 3-carbon spacer, were prepared as shown in Schemes 28-37. Analogs 45a and 45b contain two identical aryl rings separated by an unsaturated 3-carbon spacer having a single carbonyl and were designed to test the importance of the length of the spacer. Analog 45a was prepared as shown in Scheme 28 following the procedures described by Masuda (Masuda et al., Phytochemistry 1993, 32(6), 1557-1560) and by Kohler and Chadwell (Kohler et al., Org. Synth., Coll. Vol. 1 1932, 78-80). 4-Hydroxy-3-methoxyacetophenone (44a) was reacted with potassium carbonate and chloromethyl methyl ether (18) in a substitution reaction to give compound 44j. The formation of the product was verified by proton NMR by the appearance of signals at 3.33 ppm and 5.12 ppm for the protons of the protecting group. Compound 1j, prepared as shown in Scheme 12, was reacted with compound 44j and barium hydroxide in an aldol reaction to give compound 45j. The formation of the product was verified by proton NMR by the appearance of a pair of doublets in the aromatic region with J values of 15.5 Hz for the alkene protons on the spacer. Also observed in the proton NMR was the loss of a signal at 2.38 ppm for the methyl protons of the starting acetophenone (44j) and the loss of a signal at 9.75 ppm for the aldehyde proton of the starting benzaldehyde (1j). Compound 45j was then reacted with a catalytic amount of concentrated hydrochloric acid to give the phenol, analog 45a. The formation of the product was verified by proton NMR by the appearance of signals at 6.00 ppm and 6.19 ppm for the phenolic protons. Also observed in the proton NMR was the loss of signals at 3.50 ppm, 5.25 ppm 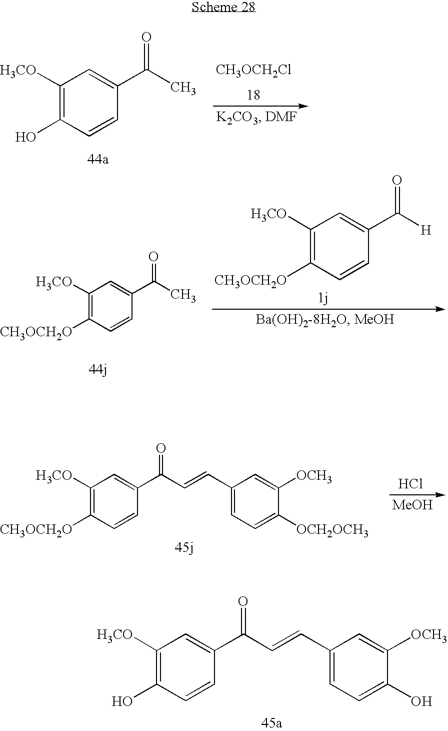
and 5.30 ppm for the protons in the protecting groups in compound 45j. The structure was also verified by carbon NMR by the loss of signals at ˜57 ppm and ˜95 ppm for the carbons of the protecting groups in compound 45j.

Scheme 29 describes the synthesis of analog 45b following the procedure described by Kohler and Chadwell (Kohler et al., Org. Synth., Coll. Vol. 1 1932, 78-80). Acetophenone (44b) was reacted with benzaldehyde (1b) and sodium hydroxide in an aldol reaction to give analog 45b. The formation of the product was verified by proton NMR by the appearance of a pair of doublets in the aromatic region with J values of 15.7 Hz for the alkene protons on the spacer. Also observed in the proton NMR was the loss of a signal at 9.74 ppm for the aldehyde proton on the starting benzaldehyde (1b) and a signal at 2.51 ppm for the methyl protons in the starting acetophenone (44b). The structure was also verified by carbon NMR by the appearance of two signals in the aromatic region for the alkene carbons on the spacer and the loss of a signal at 26.0 ppm for the methyl carbon on the starting acetophenone (44b).

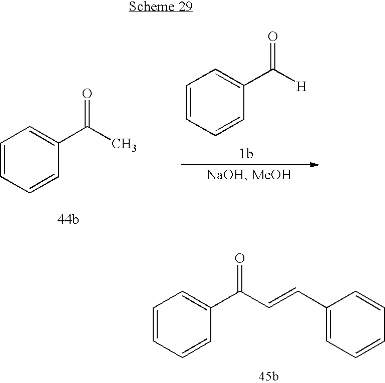

Six additional 3-carbon spacer analogs, 46a, 46ak-46am, 48a and 48ad, were prepared as shown in Schemes 30-34. Analogs 46a, 46ak-46am, 48a and 48ad contain two different aryl rings separated by an unsaturated 3-carbon spacer having a single carbonyl. These analogs were designed to test the importance of the length of the spacer and the importance of ring symmetry in series 3 analogs. Analog 46a was prepared as shown in Scheme 30 following the procedures described by Masuda (Masuda et al., Phytochemistry 1993, 32(6), 1557-1560) and by Kohler and Chadwell (Kohler et al., Org. Synth., Coll. Vol. 1 1932, 78-80). Compound 44j, prepared as shown in Scheme 28, was reacted with benzaldehyde (1b) and barium hydroxide in an aldol reaction to give compound 46j. The formation of the product was verified by proton NMR by the appearance of a pair of doublets in the aromatic region with J values of 15.7 Hz for the alkene protons on the spacer. Also observed in the proton NMR was the loss of a signal at 2.38 ppm for the methyl protons on the starting acetophenone (44j) and a signal at 9.74 ppm for the aldehyde proton in the starting benzaldehyde (1b). Compound 46j was then reacted with a catalytic amount of concentrated hydrochloric acid to give the phenol, analog 46a. The formation of the product was verified by proton NMR by the appearance of a signal at 6.29 ppm for the phenolic proton. Also observed in the proton NMR was the loss of signals at 3.48 ppm and 5.28 ppm for the protons of the protecting group in compound 46j. The structure was also verified by carbon NMR by the loss of signals at ˜57 ppm and ˜95 ppm for the carbons of the protecting group in compound 46j.

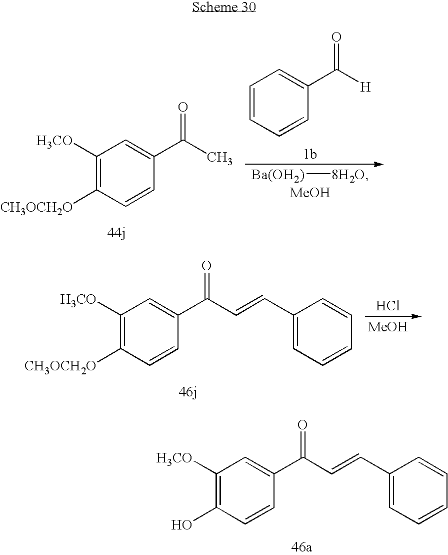

Scheme 31 describes the synthesis of analogs 46ak and 46al following the procedure described by Kohler and Chadwell (Kohler et al., Org. Synth., Coll. Vol. 1 1932, 78-80). Acetophenone 44ak or 44al was reacted with benzaldehyde (1b) and barium hydroxide in an aldol reaction to give analogs 46ak or 46al respectively. The formation of the products was verified by proton NMR by the appearance of a pair of doublets in the aromatic region with J values of 15.9-16.1 Hz for the alkene protons on the spacer. Also observed in the proton NMR was the loss of a signal at 9.74 ppm for the aldehyde proton in the starting benzaldehyde (1b) and a signal at ˜2.49 ppm for the methyl protons of the starting acetophenones (44ak and 44al). The structure of the product was also verified by carbon NMR by the appearance of two signals in the aromatic region for the alkene carbons on the spacer. Also observed in the carbon NMR was the loss of a signal at ˜26.7 ppm for the methyl carbon on the starting acetophenones (44ak and 44al).

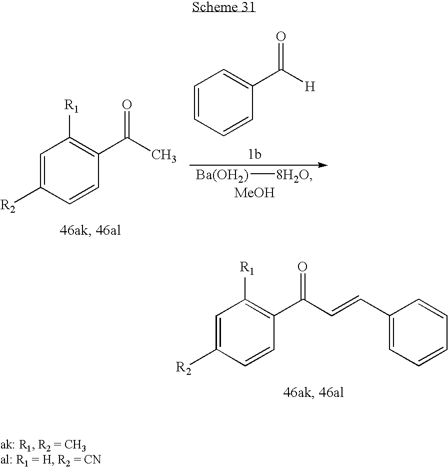

Scheme 32 describes the synthesis of analog 46ad following the procedure described by Cleeland (Cleeland et al., U.S. Pat. No. 4,045,487 (1977); Chem. Abstr., 87, P167872u (1977)). Compound 44al was reacted with concentrated sulfuric acid in a hydrolysis reaction to give compound 44ad. The formation of the product was verified by proton NMR by the appearance of a signal at 13.34 ppm for the carboxylic acid proton. Compound 44ad was then reacted with benzaldehyde (1b) and sodium hydroxide in an aldol reaction followed by acidification with dilute hydrochloric acid to give analog 46ad. The formation of the product was verified by proton NMR by the appearance of a pair of doublets in the aromatic region with J values of 15.5-16.1 Hz for the alkene protons on the spacer. Also observed in the proton NMR was the loss of a signal at 2.43 ppm for the methyl protons on the starting acetophenone (44ad) and a signal at 9.74 ppm for the aldehyde proton in the starting benzaldehyde (1b). The structure of the product was also verified by carbon NMR by the appearance of two signals in the aromatic region for the alkene carbons on the spacer. Also seen in the carbon NMR was the loss of a signal at 23.7 ppm for the methyl carbon in the starting acetophenone (44ad).

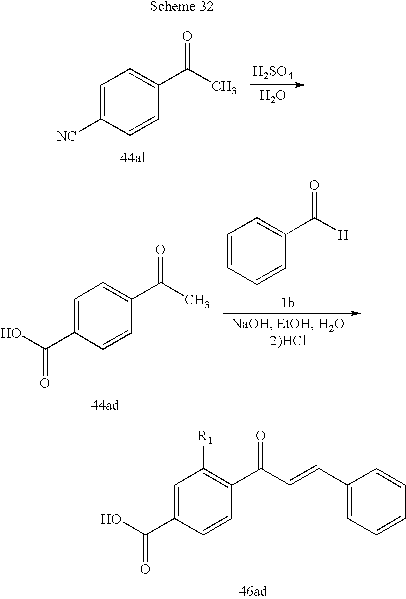

Scheme 33 describes the synthesis of analog 48a following the procedure described by Takagaki (Takagaki et al., European Patent 370,461 (1990); Chem. Abstr., 113, P230963x (1990)). Compound 1a was reacted with 3,4-dihydropyran and pyridinium p-toluenesulfonate in a substitution reaction to give compound 1am. The formation of the product was verified by proton NMR by the appearance of multiplets in the aliphatic region for the protecting group protons. Compound 1am was then reacted with acetophenone (44b) and barium hydroxide in an aldol reaction to give compound 48am. The formation of the product was verified by proton NMR by the appearance of a pair of doublets in the aromatic region with J values of 15.5-15.9 Hz for the alkene protons on the spacer. Also observed in the proton NMR was the loss of a signal at 2.38 ppm for the methyl protons on the starting acetophenone (44b) and a signal at 9.87 ppm for the aldehyde proton in the starting benzaldehyde (1am). Compound 48am was then reacted with p-toluenesulfonic acid to give the phenol, analog 48a. The formation of the product was verified by proton NMR by the appearance of a signal at 5.96 ppm for the phenolic proton. Also observed in the proton NMR was the loss of multiplets in the aliphatic region for the protons of the protecting group in compound 48am. The structure was also verified by carbon NMR by the loss of five signals in the aliphatic region for the carbons of the protecting group in compound 48am.

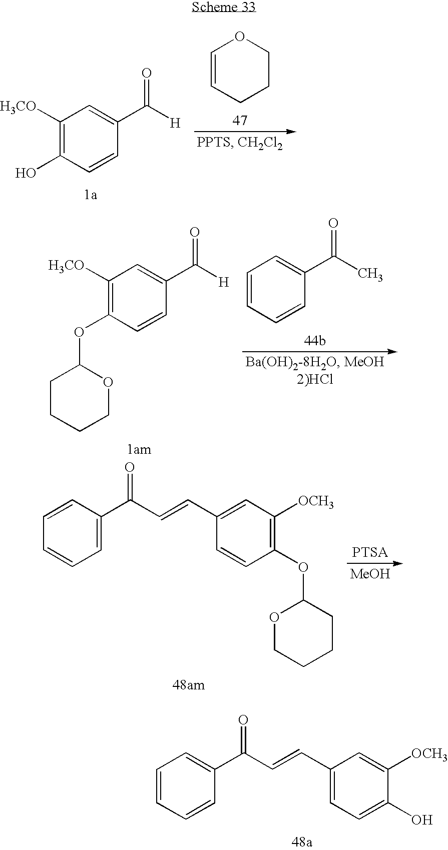

Scheme 34 describes the synthesis of analog 48ad following the procedure described by Cleeland (Cleeland et al., U.S. Pat. No. 4,045,487 (1977); Chem. Abstr., 87, P167872u (1977)). Compound 1ad was reacted with compound 44b and sodium hydroxide in an aldol reaction followed by acidification with dilute hydrochloric acid to give analog 48ad. The formation of the product was verified by proton NMR by the appearance of a pair of doublets in the aromatic region for the alkene protons on the spacer. Also observed in the proton NMR was the loss of a signal at 2.38 ppm for the methyl protons on the starting acetophenone (44b) and a signal at 10.12 ppm for the 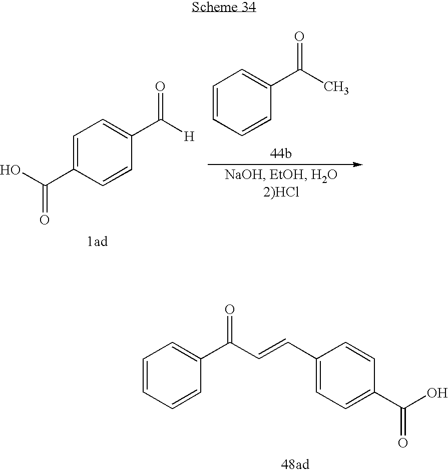
aldehyde proton in the starting benzaldehyde (1ad). The structure of the product was also verified by carbon NMR by the appearance of two signals in the aromatic region for the alkene carbons on the spacer. Also seen in the carbon NMR was the loss of a signal at 26.0 ppm for the methyl carbon in the starting acetophenone (44b).

An additional 3-carbon spacer analog, 50b, was prepared as shown in Scheme 35 following the procedure described by Chisolm (Chisolm et al., U.S. Pat. No. 050,713 (1992); Chem. Abstr., 115, P207660d (1992)). Analog 50b contains two aryl rings separated by a 3-carbon spacer having two carbonyls. This analog was designed to test the importance of two carbonyls in a 3-carbon spacer. Acetophenone (44b) was reacted with methyl benzoate (49) and sodium methoxide in a condensation reaction to give analog 50b. The formation of the product was verified by proton NMR by the appearance of a signal at 6.85 ppm for the enol proton on the spacer and the loss of a signal at 2.38 for the methyl protons on acetophenone (44b) and a signal at 3.88 ppm for the methyl ester protons of methyl benzoate (49). The structure was also verified by carbon NMR by the appearance of signals at 93.1 ppm for the enol carbon and 185.6 ppm for the carbonyl carbons on the spacer. Also observed in the carbon NMR was the loss of signals at 166.2 ppm for the carbonyl carbon and 51.4 ppm for the methyl carbon on methyl benzoate (49) and signals at 197.3 ppm for the carbonyl carbon and 26.0 for the methyl carbon on acetophenone (44b).

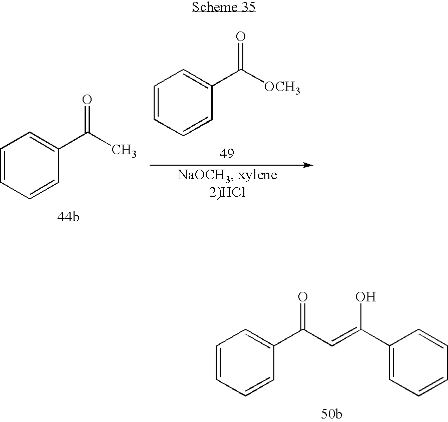

Six additional analogs, 52b, 52c, 52e, 52aa, 52ac and 53 were prepared as shown in Schemes 36 and 37 following the procedure described by Selvaraj (Selvaraj et al., Ind. J. Chem., Sect. B 1987, 26B, 1104-1105). Analogs 52b, 52c, 52e, 52aa, 52ac and 53 contain two identical aryl rings separated by a 3-carbon spacer having both a carbonyl and a saturated heterocyclic ring and were designed to test the importance of a heterocyclic ring in the spacer. Analogs 52b, 52c, 52e, 52aa and 52ac were prepared as shown is Scheme 36 by reaction of analogs 20b, 20c, 20e, 20aa and 20ac with methylamine (51) in a Michael addition reaction. The formation of the products was verified by proton NMR by the appearance of a pair of doublets at ˜2.50 ppm and ˜3.45 ppm for the protons alpha to the carbonyl and a triplet at ˜2.82 ppm for the protons alpha to the amine. The structures were also verified by carbon NMR by the appearance of signals at ˜50.8 ppm and ˜70.2 ppm for the alkane carbons in the nitrogen containing heterocyclic ring and by the loss of two signals in the aromatic region for the alkene carbons. Analogs 52c and 52ac, which are not in the literature, were verified by high resolution mass spectroscopy.

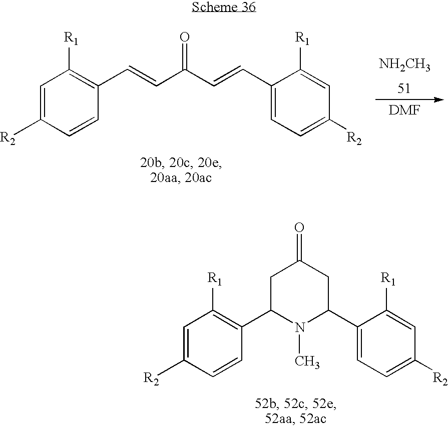

Scheme 37 describes the synthesis of analog 53 following the procedure described by Selvaraj (Selvaraj et al., Ind. J. Chem., Sect. B 1987, 26B, 1104-1105). Analog 25 was reacted with methylamine (51) in a Michael addition reaction to give analog 53. The formation of the products was verified by proton NMR by the appearance of a pair of doublets at 2.59 ppm and 3.66 ppm for the protons alpha to the carbonyl and a triplet at 2.97 ppm for the protons alpha to the amine. The structures were also verified by carbon NMR by the appearance of signals at 50.7 ppm and 70.3 ppm for the alkane carbons in the nitrogen containing heterocyclic ring and by the loss of two signals in the aromatic region for the alkene carbons. Analog 53, which is not in the literature, was verified by high resolution mass spectroscopy. 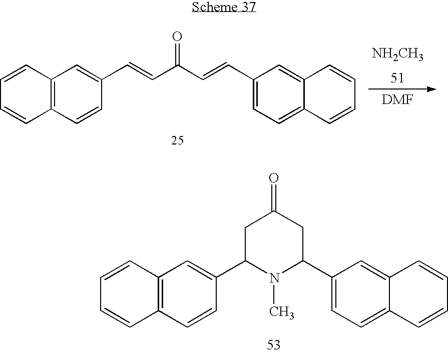
Experimental

Reagent quality solvents were used without purification with the exception of ethyl acetate which was distilled from magnesium sulfate before use. Liquid benzaldehydes, acetone and acetyl acetone were distilled before use. All other reagents were obtained from commercial sources and used without further purification. All compounds isolated were greater than 95% pure by proton and carbon NMR. Column chromatographic separations were performed using EM Science type 60 silica gel (230-400 mesh). Melting points were determined on a Thomas Hoover capillary melting point apparatus and are uncorrected. NMR spectra were recorded on a Bruker AC250 (250 MHz) NMR spectrometer in CDCl₃ unless otherwise noted. Chemical shifts are reported in ppm (δ) relative to CDCl₃ at 7.24 ppm for proton NMR and 77.0 for carbon NMR or DMSO at 2.49 ppm for proton NMR and 39.5 ppm for carbon NMR. Proton NMR peaks are reported as follows: chemical shift, multiplicity (s=singlet, d=doublet, t=triplet, q=quartet, m=multiplet, dd=doublet of doublets and dt=doublet of triplets), integration, and coupling constants (J in Hz). High resolution mass spectra were performed at the UNM Mass Spectrometry Facility, University of New Mexico, Albuquerque N. Mex. Analytical data was obtained from Galbraith Laboratories, Knoxville Tenn.

4-Methoxymethyloxy-3-methoxybenzaldehyde (1j). 4-Hydroxy-3-methoxybenz-aldehyde (1a, 2.00 g, 13.1 mmol) and potassium carbonate (9.00 g, 65.1 mmol) were combined in dimethyl formamide (30 ml) and stirred for 15 min at room temperature. Chloromethyl methyl ether (1.60 ml, 21.1 mmol) was added and stirring was continued for 6 hr at room temperature. The resulting mixture was filtered and the filtrate extracted into ethyl acetate, washed with saturated sodium chloride, dried over magnesium sulfate, filtered and evaporated to give 2.55 g (99%) of a white solid: mp 39-40° C. [expected mp 41° C.]; ¹H NMR: δ 3.40 (s, 3H), 3.83 (s, 3H), 5.21 (s, 2H), 7.15 (d, 1H, J=8.7 Hz), 7.30 (dd, 1H, J=6.0, 2.0 Hz), 7.32 (s, 1H), 9.75 (s, 1H); ¹³C NMR: δ 55.8, 56.2, 94.8, 109.4, 114.6, 125.9, 130.9, 149.8, 151.7, 190.4.

4-Carbmethoxybenzaldehyde (1ae). 4-Formylbenzoic acid (1ad, 1.00 g, 6.7 mmol) was dissolved in dry methanol (200 ml) and stirred for 10 min at 0° C. Thionyl chloride (6 ml, 82.3 mmol) was added dropwise and the mixture stirred for 90 min at 0° C. and 3 hr at room temperature. The methanol was evaporated and the resulting residue extracted into dichloromethane, washed with saturated sodium chloride, dried over magnesium sulfate, filtered and evaporated to afford a solid. The crude solid was recrystallized from hexane to give 1.07 g (98%) of a white solid: mp 60-62° C. [expected mp 61° C.]; ¹H NMR: δ 3.87 (s, 3H), 8.01 (d, 2H, J=7.9 Hz), 8.13 (d, 2H, J=7.6 Hz), 10.08 (s, 1H); ¹³C NMR: δ 52.7, 129.7, 129.9, 134.4, 139.1, 165.6, 192.9.

1,7-Bis(4-hydroxy-3-methoxyphenyl)-1,6-heptadiene-3,5-dione (3a). Boric anhydride (0.49 g, 7.0 mmol) was combined with 2,4-pentanedione (2, 1.05 ml, 10.0 mmol) and stirred for 18 hr at room temperature under a nitrogen atmosphere. A solution of dry ethyl acetate (10 ml), 4-hydroxy-3-methoxybenzaldehyde (1a, 3.04 g, 20.0 mmol) and tributyl borate (11.00 ml, 40.5 mmol) was added and the mixture stirred for 15 min at room temperature. Butylamine (0.20 ml, 2.0 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was extracted into ethyl acetate, washed with saturated sodium chloride, dried over magnesium sulfate, filtered and evaporated to afford a solid. The crude solid was triturated with methanol to give 2.82 g (77%) of an orange-yellow solid: mp 182-184° C. [expected mp 182-183° C.]; ¹H NMR: (DMSO) δ 3.83 (s, 6H), 6.05 (s, 1H), 6.74 (d, 2H, J=15.9 Hz), 6.82 (d, 2H, J=8.1 Hz), 7.14 (d, 2H, J=8.0 Hz), 7.31 (s, 2H), 7.54 (d, 2H, J=15.7 Hz), 9.63 (s, 2H), 16.29 (s, 1H); ¹³C NMR: (DMSO) δ 55.6, 100.5, 111.3, 115.5, 120.9, 122.8, 126.2, 140.4, 147.8, 149.1, 182.8.

1,7-Diphenyl-1,6-heptadiene-3,5-dione (3b). Boric anhydride (0.49 g, 7.0 mmol) was combined with 2,4-pentanedione (2, 1.05 ml, 10.0 mmol) and stirred for 18 hr at room temperature under a nitrogen atmosphere. A solution of dry ethyl acetate (10 ml), benzaldehyde (1b, 2.05 g, 20.2 mmol) and tributyl borate (11.00 ml, 40.5 mmol) was added and the mixture stirred for 15 min at room temperature. Butylamine (0.20 ml, 2.0 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was extracted into ethyl acetate, washed with saturated sodium chloride, dried over magnesium sulfate, filtered and evaporated to afford a solid. The crude solid was triturated with methanol to give 0.90 g (33%) of a yellow solid: mp 140-142° C. [expected mp 139-140° C.]; ¹H NMR: δ 5.84 (s, 1H), 6.62 (d, 2H, J=15.7 Hz), 7.39 (m, 6H), 7.54 (dd, 4H, J=7.4, 4.0 Hz), 7.66 (d, 2H, J=15.9 Hz), 15.85 (s, 1H); ¹³C NMR: δ 101.6, 124.1, 128.0, 128.9, 130.0, 135.0, 140.5, 183.2.

1,7-Bis(2-methoxyphenyl)-1,6-heptadiene-3,5-dione (3c). Boric anhydride (0.33 g, 4.7 mmol) was combined with 2,4-pentanedione (2, 0.70 ml, 6.7 mmol) and stirred for 18 hr at room temperature. A solution of dry ethyl acetate (15 ml), 2-methoxybenzaldehyde (1c, 1.81 g, 13.3 mmol) and tributyl borate (7.25 ml, 26.7 mmol) was added and the mixture stirred for 15 min at room temperature. Butylamine (1.00 ml, 10.1 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was extracted into ethyl acetate, washed with saturated sodium chloride, dried over magnesium sulfate, filtered and evaporated to afford a semi-solid. The crude semi-solid was chromatographed on silica gel with ethyl acetate/hexane to give 0.48 g (21%) of a yellow crystals: mp 121-123° C. [expected mp 121-122° C.]; ¹H NMR: δ 3.89 (s, 6H), 5.86 (s, 1H), 6.71 (d, 2H, J=16.1 Hz), 6.94 (m, 4H), 7.33 (dt, 2H, J=8.1, 1.4 Hz), 7.54 (dd, 2H, J=7.8, 1.4 Hz), 7.97 (d, 2H, J=16.1 Hz), 16.00 (s, 1H); ¹³C NMR: δ 55.5, 101.4, 111.1, 120.6, 124.0, 124.7, 128.5, 131.1, 135.6, 158.3, 183.6.

1,7-Bis(2,3-dimethoxyphenyl)-1,6-heptadiene-3,5-dione (3d). Boric anhydride (0.49 g, 7.0 mmol) was combined with 2,4-pentanedione (2, 1.05 ml, 10.0 mmol) and stirred for 18 hr at room temperature under a nitrogen atmosphere. A solution of dry ethyl acetate (10 ml), 2,3-dimethoxybenzaldehyde (1d, 3.32 g, 20.0 mmol) and tributyl borate (11.00 ml, 40.5 mmol) was added and the mixture stirred for 15 min at room temperature. Butylamine (0.20 ml, 2.0 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was extracted into ethyl acetate, washed with saturated sodium chloride, dried over magnesium sulfate, filtered and evaporated to afford a semi-solid. The crude semi-solid was triturated with methanol to give 2.01 g (51%) of a yellow solid: mp 117-120° C. [expected mp 117-120° C.]; ¹H NMR: δ 3.87 (s, 12H), 5.87 (s, 1H), 6.68 (d, 2H, J=16.1 Hz), 6.92 (d, 2H, J=8.2 Hz), 7.02 (t, 2H, J=8.0 Hz), 7.18 (d, 2H, J=6.8 Hz), 7.95 (d, 2H, J=16.1 Hz), 15.88 (s, 1H); ¹³C NMR: (DMSO) δ 55.7, 60.7, 101.9, 114.6, 118.7, 124.1, 125.1, 128.0, 134.2, 147.7, 152.6, 182.9; Anal. Calcd for C₂₃H₂₄O₆: C, 69.68; H, 6.10. Found: C, 69.43; H, 6.16.

1,7-Bis(4-methoxyphenyl)-1,6-heptadiene-3,5-dione (3e). Boric anhydride (0.49 g, 7.0 mmol) was combined with 2,4-pentanedione (2, 1.05 ml, 10.0 mmol) and stirred for 18 hr at room temperature under a nitrogen atmosphere. A solution of dry ethyl acetate (10 ml), 4-methoxybenzaldehyde (1e, 2.43 ml, 20.0 mmol) and tributyl borate (11.00 ml, 40.5 mmol) was added and the mixture stirred for 15 min at room temperature. Butylamine (0.20 ml, 2.0 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was extracted into ethyl acetate, washed with saturated sodium chloride, dried over magnesium sulfate, filtered and evaporated to afford a solid. The crude solid was triturated with methanol to give 2.83 g (84%) of a yellow solid: mp 157-159° C. [expected mp 154-155° C.]; ¹H NMR: δ 3.82 (s, 6H), 5.75 (s, 1H), 6.48 (d, 2H, J=15.9 Hz), 6.90 (d, 4H, J=8.7 Hz), 7.49 (d, 4H, J=8.7 Hz), 7.60 (d, 2H, J=15.9 Hz), 16.04 (s, 1H); ¹³C NMR: δ 55.4, 101.2, 114.4, 121.9, 127.9, 129.7, 140.0, 161.2, 183.2.

1,7-Bis(4-hydroxyphenyl)-1,6-heptadiene-3,5-dione (3f). Boric anhydride (0.33 g, 4.7 mmol) was combined with 2,4-pentanedione (2, 0.70 ml, 6.7 mmol) and stirred for 18 hr at room temperature. A solution of dry ethyl acetate (15 ml), 4-hydroxybenzaldehyde (1f, 1.62 g, 13.3 mmol) and tributyl borate (7.25 ml, 26.7 mmol) was added and the mixture stirred for 15 min at room temperature. Butylamine (1.0 ml, 10.1 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was extracted into ethyl acetate, washed with saturated sodium chloride, dried over magnesium sulfate, filtered and evaporated to afford a solid. The crude solid was recrystallized from methanol to give 0.40 g (19%) of red-orange crystals: mp 226-228° C. [expected mp 223-224° C.]; ¹H NMR: (DMSO) δ 6.03 (s, 1H), 6.67 (d, 2H, J=15.9 Hz), 6.81 (d, 4H, J=7.7 Hz), 7.55 (m, 6H), 10.03 (s, 2H), 16.37 (s, 1H); ¹³C NMR: (DMSO) δ 100.7, 115.8, 120.7, 125.7, 130.1, 140.1, 159.5, 182.9.

1,7-Bis(4-dimethylaminophenyl)-1,6-heptadiene-3,5-dione (3g). Boric anhydride (0.33 g, 4.7 mmol) was combined with 2,4-pentanedione (2, 0.70 ml, 6.7 mmol) and stirred for 18 hr at room temperature. A solution of dry ethyl acetate (15 ml), 4-dimethylaminobenzaldehyde (1g, 2.00 g, 13.4 mmol) and tributyl borate (7.25 ml, 26.7 mmol) was added and the mixture stirred for 15 min at room temperature. Butylamine (1.0 ml, 10.1 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was extracted into ethyl acetate, washed with saturated sodium chloride, dried over magnesium sulfate, filtered and evaporated to afford a solid. The crude solid was triturated with methanol to give 0.57 g (23%) of a purple solid: mp 207-208° C. [expected mp 210-212° C.]; ¹H NMR: (DMSO) δ 3.01 (s, 12H), 5.71 (s, 1H), 6.41 (d, 2H, J=15.61 Hz), 6.67 (d, 4H, J=8.02 Hz), 7.44 (d, 4H, J=8.11 Hz), 7.58 (d, 2H, J=15.65 Hz), 16.56 (s, 1H); ¹³C NMR: (DMSO) δ 39.6, 100.3, 111.7, 118.5, 122.0, 129.7, 140.3, 151.4, 182.6.

1,7-Bis(3-hydroxy-4-methoxyphenyl)-1,6-heptadiene-3,5-dione (3h). Boric anhydride (0.49 g, 7.0 mmol) was combined with 2,4-pentanedione (2, 1.05 ml, 10.0 mmol) and stirred for 18 hr at room temperature under a nitrogen atmosphere. A solution of dry ethyl acetate (10 ml), 3-hydroxy-4-methoxybenzaldehyde (1h, 3.04 g, 20.0 mmol) and tributyl borate (11.0 ml, 40.5 mmol) was added and the mixture stirred for 15 min at room temperature. Butylamine (0.20 ml, 2.0 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was filtered to afford a solid. The crude solid was triturated with methanol to give 2.60 g (71%) of a orange-yellow solid: mp 190-192° C. [expected mp 189-190° C.); ¹H NMR: (DMSO) δ 3.78 (s, 6H), 6.09, (s, 1H), 6.60 (d, 2H, J=15.9 Hz), 6.49 (d, 2H, J=8.9 Hz), 7.11 (m, 4H), 7.47 (d, 2H, J=15.9 Hz), 9.19 (s, 2H); ¹³C NMR: (DMSO) δ 55.6, 100.9, 112.0, 114.0, 121.2, 121.5, 127.5, 140.2, 146.6, 149.8, 182.8.

1,7-Bis(3,4-dimethoxyphenyl)-1,6-heptadiene-3,5-dione (3i). Boric anhydride (0.49 g, 7.0 mmol) and 2,4-pentanedione (2, 1.05 ml, 10.0 mmol) were combined in dry ethyl acetate (10 ml) and stirred for 30 min at 40° C. 3,4-Dimethoxybenzaldehyde (1i, 3.32 g, 20.0 mmol) and tributyl borate (7.90 ml, 29.1 mmol) were added and stirring was continued for 30 min at 40° C. A solution of butylamine (1.5 ml, 15.2 mmol) in dry ethyl acetate (10 ml) was added dropwise over 15 min and stirring was continued for 18 hr at 40° C. Hydrochloric acid (10 ml, 2 N) was added and the mixture stirred for 1 hr at 60° C. The resulting mixture was cooled to room temperature, extracted with ethyl acetate, washed with saturated sodium chloride, dried over magnesium sulfate, filtered and evaporated to afford a semi-solid. The crude semi-solid was chromatographed on silica gel with ethyl acetate/hexane to give a solid. The crude solid was recrystallized from methanol to give 1.16 g (29%) of an orange solid: mp 129-131° C. [expected mp 128-130° C.]; ¹H NMR: (DMSO) δ 3.79 (s, 6H), 3.81 (s, 6H), 6.10 (s, 1H), 6.82 (d, 2H, J=15.9 Hz), 7.00 (d, 2H, J=8.3 Hz), 7.25 (d, 2H, J=6.8 Hz), 7.33 (s, 2H), 7.57 (d, 2H, J=15.7 Hz), 16.32 (s, 1H); ¹³C NMR: (DMSO) δ 55.6, 100.8, 110.5, 111.7, 122.0, 122.7, 127.5, 140.2, 148.9, 150.9, 183.0.

3-Methyl-2,4-pentanedione (5). 2,4-Pentanedione (2, 6.3 ml, 60.2 mmol) and potassium carbonate (7.75 g, 56.1 mmol) were combined in acetone (12 ml) and stirred for 15 min at room temperature. Methyl iodide (4, 4.6 ml, 73.9 mmol) was added and the resulting mixture refluxed with a calcium chloride drying tube for 18 hr. An additional amount of methyl iodide (1.5 ml, 24.1 mmol) was added and reflux was continued for 2 hr. The resulting mixture was filtered and the solvent evaporated to afford a liquid. The crude liquid was distilled to give 5.33 g (78%) of a clear liquid: bp 164-170° C.; ¹H NMR: δ enol form: 1.65 (s, 6H), 1.92 (s, 3H); keto form: 1.12 (d, 3H, J=7.0 Hz), 2.00 (s, 6H), 3.52 (q, 1H, J=7.0 Hz); ¹³C NMR: δ 12.2, 12.6, 23.0, 28.4, 61.3, 104.4, 189.9, 204.5.

4-Methyl-1,7-bis(4-hydroxy-3-methoxyphenyl)-1,6-heptadiene-3,5-dione (6a). Boric anhydride (0.49 g, 7.0 mmol) was combined with 3-methyl-2,4-pentanedione (5, 1.14 g, 10 mmol) and stirred for 24 hr at room temperature under a nitrogen atmosphere. A solution of dry ethyl acetate (10 ml), 4-hydroxy-3-methoxybenzaldehyde (1a, 3.04 g, 20.0 mmol) and tributyl borate (11.0 ml, 40.5 mmol) was added and the mixture stirred for 30 min at room temperature. Butylamine (0.2 ml, 2.0 mmol) was added dropwise over 40 min and stirring was continued for 24 hr at room temperature. Hydrochloric acid (15 ml, 0.4 N) was warmed to 60° C., added to the mixture and stirring was continued for 4 hr. The resulting mixture was filtered through celite and silica gel. The filtrate was washed with saturated sodium chloride, dried over magnesium sulfate, filtered and evaporated to afford a solid. The crude solid was recrystallized three times from methanol to give 0.86 g (22%) of an orange solid: mp 180-183° C. [expected mp 180-183° C.]; ¹H NMR: δ 2.16 (s, 3H), 3.94 (s, 6H), 5.83 (s, 2H), 6.94 (m, 4H), 7.04 (d, 2H, J=1.6 Hz), 7.16 (d, 2H, J=8.0 Hz), 7.66 (d, 2H, J=15.5 Hz); ¹³C NMR: δ 11.5, 13.1, 55.2, 55.6, 55.8, 105.6, 111.4, 111.6, 115.5, 117.7, 122.1, 123.2, 123.5, 125.6, 126.6, 141.4, 143.7, 147.8, 149.1, 149.6, 182.1, 196.0; Anal. Calcd for C₂₂H₂₂O₆: C, 69.10; H, 5.80. Found: C, 69.19; H, 5.89.

4-Methyl-1,7-diphenyl-1,6-heptadiene-3,5-dione (6b). Boric anhydride (0.49 g, 7.0 mmol) was combined with 3-methyl-2,4-pentanedione (5, 1.14 g, 10.0 mmol) and stirred for 24 hr at room temperature under a nitrogen atmosphere. A solution of ethyl acetate (10 ml), benzaldehyde (1b, 2.05 ml, 20.2 mmol) and tributyl borate (11.0 ml, 40.5 mmol) was added and the mixture stirred for 15 min at room temperature. Butylarnine (0.20 ml, 2.0 mmol) was added dropwise over 30 min and stirring was continued for 24 hr at room temperature. The resulting mixture was filtered to afford a solid. The crude solid was triturated with methanol to give 1.80 g (62%) of an orange solid: mp 154-157° C. [expected mp 154-157° C.]; ¹H NMR: δ 2.17 (s, 3H), 7.12 (d, 2H, J=15.5 Hz), 7.38 (m, 6H), 7.58 (m, 4H), 7.74 (d, 2H, J=15.5 Hz); ¹³C NMR: δ 12.1, 106.2, 120.8, 182.1, 128.8, 129.9, 135.4, 141.3, 182.4. Anal. Calcd for C₂₀H₁₈O₂: C, 82.73; H, 6.25. Found: C, 82.69; H, 6.36.

3-Benzylidene-2,4-pentanedione (7). 2,4-Pentanedione (2, 4.10 ml, 39.2 mmol) and benzaldehyde (1b, 4.06 ml, 40.0 mmol) were stirred in benzene (10 ml). Piperdine (3 drops) and glacial acetic acid (6 drops) were added and the mixture refluxed with a Dean-Stark water trap for 3 hr. The resulting mixture was cooled to room temperature, extracted into ethyl ether, washed with hydrochloric acid (1 N), saturated sodium bicarbonate, hydrochloric acid (1 N) and twice with water. The organic layer was dried over magnesium sulfate, filtered and evaporated to afford an oil. The crude oil was distilled bulb to bulb to give 7.03 g (95%) of a yellow oil; [expected mp 165-167° C.]; ¹H NMR: δ 2.24 (s, 3H), 2.38 (s, 3H), 7.35 (s, 5H), 7.45 (s, 1H); ¹³C NMR: δ 26.5, 31.6, 128.9, 129.6, 130.5, 132.8, 139.6, 142.7, 196.2, 205.3.

3-Benzyl-2,4-pentanedione (8). 3-Benzylidene-2,4-pentanedione (7, 6.50 g, 34.5 mmol) and palladium on activated carbon (0.25 g, 10%) were combined in ethyl acetate (50 ml). The mixture was placed under a hydrogen atmosphere (60 psi) on a Parr apparatus for 4 hr at room temperature. The resulting mixture was filtered through celite and the solvent evaporated to afford an oil. The crude oil was distilled bulb to bulb to give 6.52 g (99%) of a clear oil; ¹H NMR: δ enol form: 2.02 (s, 6H), 3.62 (s, 2H), 7.24 (m, 5H); keto form: 2.07 (s, 6H), 3.11 (d, 2H, J=7.4 Hz), 4.01 (t, 1H, J=7.7 Hz), 7.24 (m, 5H); ¹³C NMR: δ 22.9, 29.4, 32.5, 33.8, 69.2, 107.9, 125.9, 126.3, 127.0, 128.1, 128.2, 137.7, 139.3, 191.4, 202.9.

4-Benzyl-1,7-bis(4-hydroxy-3-methoxyphenyl)-1,6-heptadiene-3,5-dione (9a). Boric anhydride (0.49 g, 7.0 mmol) was combined with 3-benzyl-2,4-pentanedione (8, 1.90 g, 10 mmol) and stirred for 18 hr at room temperature under a nitrogen atmosphere. A solution of dry ethyl acetate (15 ml), 4-hydroxy-3-methoxybenzaldehyde (1a, 3.04 ml, 20.0 mmol) and tributyl borate (11.0 ml, 40.5 mmol) was added and the mixture stirred for 30 min at room temperature. Butylamine (0.2 ml, 2.0 mmol) was added dropwise over 40 min and stirring was continued for 48 hr at room temperature. Hydrochloric acid (15 ml, 0.5 N) was warmed to 60° C., added to the mixture and stirring was continued for 1 hr. The resulting mixture was extracted into ethyl acetate, washed with saturated sodium chloride, dried over magnesium sulfate, filtered and evaporated to afford a solid. The crude solid was recrystallized three times from methanol to give 2.73 g (59%) of a orange-yellow solid: mp 144-146° C. [expected mp 139-141° C.]; ¹H NMR: (DMSO) δ 3.81 (s, 6H), 4.11 (s, 2H), 6.78 (d, 2H, J=8.1 Hz), 7.20 (m, 11H), 7.58 (d, 2H, J=15.1 Hz), 9.66 (s, 2H); ¹³C NMR: (DMSO) δ 30.2, 33.7, 55.6, 55.7, 63.0, 109.9, 111.3, 115.5, 117.9, 122.4, 123.2, 123.7, 125.5, 125.7, 126.0, 126.5, 127.7, 128.1, 128.3, 128.7, 139.1, 141.7, 142.3, 144.1, 147.8, 149.2, 149.7, 183.0, 194.0; Exact mass calcd for C₂₈H₂₆O₆: 458.1729, observed (M+H) 459.1798.

4-Benzyl-1,7-diphenyl-1,6-heptadiene-3,5-dione (9b). Boric anhydride (0.49 g, 7.0 mmol) was combined with 3-benzyl-2,4-pentanedione (8, 1.90 g, 10.0 mmol) and stirred for 48 hr at room temperature under a nitrogen atmosphere. A solution of dry ethyl acetate (10 ml), benzaldehyde (1b, 2.05 ml, 20.2 mmol) and tributyl borate (11.0 ml, 40.5 mmol) was added and the mixture stirred for 15 min at room temperature. Butylamine (0.20 ml, 2.0 mmol) was added dropwise over 30 min and stirring was continued for 18 hr at room temperature. Hydrochloric acid (15 ml, 0.4 N) was warmed to 60° C., added to the mixture and stirring was continued for 1 hr. The resulting mixture was filtered to afford a solid. The crude solid was triturated with methanol to give 2.30 g (63%) of a yellow solid: mp 162-164° C. [expected mp 156-158° C.]; ¹H NMR: δ 3.99 (s, 2H), 6.99 (d, 2H, J=15.6 Hz), 7.34 (m, 15H), 7.77 (d, 2H, J=15.2 Hz); ¹³C NMR: δ 31.8, 109.3, 120.8, 126.5, 127.8, 128.1, 128.8, 130.0, 135.3, 140.5, 141.9, 183.6.

4,4-Dimethyl-1,7-diphenyl-1,6-heptadiene-3,5-dione (11b). 1,7-Diphenyl-1,6-heptadiene-3,5-dione (3b, 0.30 g, 1.1 mmol) was stirred in dichloromethane (10 ml) for 5 min at room temperature. A solution of sodium hydroxide (0.10 g, 2.5 mmol), tetrabutylammonium chloride (0.42 g, 1.5 mmol) and water (3 ml) was added and the mixture stirred for 10 min at room temperature. Methyl iodide (4, 0.21 ml, 3.4 mmol) was added and the mixture stirred for 1 hr at 40° C. The mixture was cooled to room temperature, washed with saturated sodium chloride, dried over magnesium sulfate, filtered and evaporated to afford an oil. The crude oil was distilled bulb to bulb to give 0.25 g (76%) of a yellow oil; ¹H NMR: δ 1.46 (s, 6H), 6.77 (d, 2H, J=15.7 Hz), 7.33 (m, 6H), 7.49 (m, 4H), 7.72 (d, 2H, J=15.6 Hz); ¹³C NMR: δ 21.1, 60.9, 121.4, 128.5, 128.7, 130.6, 134.1, 144.1, 197.9; Anal. Calcd for C₂₀H₁₈O₂: C, 82.86; H, 6.62. Found: C, 82.54; H, 6.72.

4,4-Dibenzyl-1,7-diphenyl-1,6-heptadiene-3,5-dione (12b). 1,7-Diphenyl-1,6-heptadiene-3,5-dione (3b, 0.25 g, 0.9 mmol) was stirred in dichloromethane (4 ml) for 5 min at room temperature. A solution of sodium hydroxide (80.0 mg, 2.0 mmol), tetrabutylammonium chloride (0.29 g, 1.0 mmol) and water (2 ml) was added and the mixture stirred for 10 min at room temperature. Benzyl bromide (10, 0.22 ml, 1.8 mmol) was added and the mixture stirred for 1 hr at 40° C. The resulting mixture was cooled to room temperature, washed with saturated sodium chloride, dried over magnesium sulfate, filtered and evaporated to afford a solid. The crude solid was chromatographed on silica gel with ethyl acetate/hexane to give a solid. The solid was recrystallized from methanol to give 0.25 g (61%) of a white solid: mp 182-183° C. [expected mp 181° C.]; ¹H NMR: δ 3.39 (s, 4H), 6.70 (d, 2H, J=15.5 Hz), 7.09-7.44 (m, 20H), 7.73 (d, 2H, J=15.5 Hz); ¹³C NMR: δ 37.7, 70.3, 123.1, 126.7, 128.1, 128.6, 128.8, 130.3, 130.7, 134.2, 136.3, 142.7, 196.8.

1,7-Bis(4-hydroxy-3-methoxyphenyl)heptane-3,5-dione (13a). 1,7-Bis(4-hydroxy-3-methoxyphenyl)-1,6-heptadiene-3,5-dione (3a, 0.55 g, 1.5 mmol) and palladium on activated carbon (0.25 g, 5%) were combined in ethyl acetate (30 ml). The mixture was placed under a hydrogen atmosphere (60 psi) on a Parr apparatus for 4 hr at room temperature. The resulting mixture was filtered through celite and the solvent evaporated to afford a solid. The crude solid was recrystallized from ethyl acetate/hexane to give 0.30 g (54%) of white crystals: mp 92-94° C. [expected mp 92-93° C.]; ¹H NMR: δ 2.53 (t, 4H, J=7.9 Hz), 2.83 (m, 4H), 3.84 (s, 6H), 5.40 (s, 1H), 5.48 (s, 2H), 6.64 (m, 4H), 6.81 (d, 2H, J=8.3 Hz), 15.44 (s, 1H); ¹³C NMR: δ 29.2, 31.3, 40.4, 45.5, 55.9, 99.7, 110.9, 111.0, 114.3, 120.7, 132.4, 143.9, 146.3, 193.0.

1,7-Diphenylheptane-3,5-dione (13b). 1,7-Diphenyl- 1,6-heptadiene-3,5-dione (3b, 0.56 g, 2.0 mmol) and palladium on activated carbon (0.25 g, 5%) were combined in ethyl acetate (40 ml). The mixture was placed under a hydrogen atmosphere (60 psi) on a Parr apparatus for 4 hr at room temperature. The resulting mixture was filtered through celite and the solvent evaporated to afford an oil. The crude oil was purified by preparative thin layer chromatography with ethyl acetate/hexane to give 0.40 g (70%) of an orange-yellow oil; ¹H NMR: δ enol form: 2.53 (m, 4H), 2.83 (m, 4H), 5.47 (s, 1H), 7.30 (m, 10H); keto form: 2.53 (m, 4H), 2.83 (m, 4H), 3.54 (s, 2H), 7.30 (m, 10H); ¹³C NMR: δ 29.4, 31.5, 39.9, 45.0, 99.5, 126.1, 128.3, 128.5, 140.5, 170.4, 172.0, 192.8.

4-Methyl-1,7-bis(4-hydroxy-3-methoxyphenyl)heptane-3,5-dione (14a). 4-Methyl-1,7-bis(4-hydroxy-3-methoxyphenyl)-1,6-heptadiene-3,5-dione (6a, 0.20 g, 0.5 mmol) and palladium on activated carbon (0.25 g, 10%) were combined in ethyl acetate (100 ml). The mixture was placed under a hydrogen atmosphere (60 psi) on a Parr apparatus for 2 hr at room temperature. The resulting mixture was filtered through celite and the solvent evaporated to afford an oil. The crude oil was twice chromatographed on silica gel with ethyl acetate/hexane to give a semi-solid. The crude semi-solid was distilled bulb to bulb to give 80 mg (38%) of a pale yellow oil; ¹H NMR: δ enol form: 1.69 (s, 3H), 2.72 (m, 8H), 3.83 (s, 6H), 5.48 (s, 2H), 6.69 (m, 4H), 6.79 (d, 2H, J=7.5 Hz); keto form: 1.23 (d, 3H, J=7.2 Hz), 2.72 (m, 8H), 3.57 (q, 1H, J=7.0 Hz), 3.83 (s, 6H), 5.48 (s, 2H), 6.69 (m, 4H), 6.79 (d, 2H, J=7.5 Hz); ¹³C NMR: δ 12.5, 29.2, 43.3, 55.9, 61.4, 111.0, 114.2, 120.7, 132.4, 143.9, 146.3, 206.1; Exact mass calcd for C₂₂H₂₆O₆: 386.1729, observed (M+H) 387.1783.

4-Methyl-1,7-diphenylheptane-3,5-dione (14b). 4-Methyl-1,7-diphenyl-1,6-hetpadiene-3,5-dione (6b, 0.96 g, 3.3 mmol) and palladium on activated carbon (0.25 g, 10%) were combined in ethyl acetate (50 ml). The mixture was placed under a hydrogen atmosphere (60 psi) on a Parr apparatus for 2 hr at room temperature. The resulting mixture was filtered through celite and the filtrate was washed with saturated sodium chloride, dried over magnesium sulfate, filtered and evaporated to afford an oil. The crude oil was twice chromatographed on silica gel with ethyl acetate/hexane to give an oil. The oil was distilled bulb to bulb to give 0.71 g (73%) of a clear oil; ¹H NMR: δ enol form: 1.66 (s, 3H), 2.73 (m, 8H), 7.17 (m, 10H); keto form: 1.20 (d, 3H, J=7.2 Hz), 2.73 (m, 8H), 3.55 (q, 1H, J=7.0 Hz), 7.17 (m, 10H); ¹³C NMR: ∂ 12.3, 29.3, 31.0, 37.6, 42.8, 60.9, 104.2, 125.9, 128.1, 128.2, 140.4, 140.8, 174.6, 191.5, 205.6; Exact mass calcd for C₂₀H₂₂O₂: 294.1620, observed (M+H) 295.1693.

4-Benzyl-1,7-bis(4-hydroxy-3-methoxyphenyl)heptane-3,5-dione (15a). 4-Benzyl-1,7-bis(4-hydroxy-3-methoxyphenyl)-1,6-heptadiene-3,5-dione (9a, 0.25 g, 0.5 mmol) and palladium on activated carbon (0.20 g, 10%) were combined in ethyl acetate (45 ml). The mixture was placed under a hydrogen atmosphere (60 psi) on a Parr apparatus for 2 hr at room temperature. The resulting mixture was filtered through celite and the filtrate was washed with saturated sodium chloride, dried over magnesium sulfate, filtered and evaporated to afford an oil. The crude oil was twice chromatographed on silica gel with ethyl acetateihexane to give 0.12 g (48%) of a pale yellow oil; ¹H NMR: δ enol form: 2.66 (m, 8H), 3.53 (s, 2H), 3.77 (s, 6H), 5.55 (s, 2H), 6.56 (m, 4H), 6.77 (d, 2H, J=7.6 Hz), 7.06 (m, 6H), 7.20 (m, 2H); keto form: 2.66 (m, 8H), 3.08 (d, 2H, J=7.3 Hz), 3.82 (s, 6H), 3.92 (t, 1H, J=8.3 Hz), 5.55 (s, 2H), 6.56 (m, 4H), 6.77 (d, 2H, J=7.6 Hz), 7.06 (m, 6H), 7.20 (m, 2H); ¹³C NMR: δ 29.0, 31.1, 31.8, 34.3, 37.7, 44.6, 55.8, 69.2, 111.0, 114.2, 120.7, 120.8, 126.6, 127.4, 128.5, 128.6, 132.3, 132.6, 137.9, 143.8, 146.3, 193.4, 204.5; Exact mass calcd for C₂₈H₃₀O₆: 462.2042, observed (M+H) 463.2073.

4-Benzyl-1,7-diphenylheptane-3,5-dione (15b). 4-Benzyl-1,7-diphenyl-1,6-heptadiene-3,5-dione (9b, 0.26 g, 0.7 mmol) and palladium on activated carbon (0.25 g, 10%) were combined in ethyl acetate (50 ml). The mixture was placed under a hydrogen atmosphere (60 psi) on a Parr apparatus for 2 hr at room temperature. The resulting mixture was filtered through celite and the filtrate was washed with saturated sodium chloride, dried over magnesium sulfate, filtered and evaporated to afford an oil. Hexane was added to the crude oil and the resulting precipitate was filtered. The crude solid was recrystallized twice from hexane to give 0.18 g (69%) of white needles: mp 74-75° C.; ¹H NMR: 8 enol form: 2.61 (m, 10H), 7.14 (m, 15H); keto form: 2.61 (m, 8H), 3.07 (d, 2H, J=7.2 Hz), 3.90 (t, 1H, J=7.6 Hz), 7.14 (m, 15H); ¹³C NMR: δ 29.3, 34.3, 44.3, 69.2, 126.1, 126.7, 128.3, 128.4, 128.6, 128.7, 137.9, 140.4, 204.3; Exact mass calcd for C₂₆H₂₆O₂: 370.1933, observed (M+H) 371.2014.

4,4-Dimethyl-1,7-diphenylheptane-3,5-dione (16b). 4,4-Dimethyl-1,7-diphenyl-1,6-heptadiene-3,5-dione (11b, 0.15 g, 0.5 mmol) and palladium on activated carbon (0.20 g, 10%) were combined in ethyl acetate (25 ml). The mixture was placed under a hydrogen atmosphere (60 psi) on a Parr apparatus for 1 hr at room temperature. The resulting mixture was filtered through celite and the filtrate was washed with saturated sodium chloride, dried over magnesium sulfate, filtered and evaporated to afford an oil. The crude oil was chromatographed on silica gel with ethyl acetate/hexane to give 0.12 g (80%) of a pale yellow oil; ¹H NMR: δ 1.25 (s, 6H), 2.60 (t, 4H, J=7.4 Hz), 2.80 (t, 4H, J=7.0 Hz), 7.18 (m, I10H); ¹³C NMR: δ 21.1, 29.8, 40.2, 62.4, 126.1, 128.3, 140.7, 208.4; Exact mass calcd for C₂₁H₂₄O₂: 308.1776, observed (M+H) 309.1843.

4,4-Dibenzyl-1,7-diphenylheptane-3,5-dione (17b). 4,4-Dibenzyl-1,7-diphenyl-1,6-heptadiene-3,5-dione (12b, 70 mg, 0.2 mmol) and palladium on activated carbon (0.10 g, 10%) were combined in ethyl acetate (25 ml). The mixture was placed under a hydrogen atmosphere (60 psi) on a Parr apparatus for 5 hr at room temperature. The resulting mixture was filtered through celite and the filtrate was washed with saturated sodium chloride, dried over magnesium sulfate, filtered and evaporated to afford a solid. The crude solid was chromatographed on silica gel with ethyl acetate/hexane to give a solid. The solid was recrystallized from hexane to give 60 mg (86%) of a white solid: mp 101-102° C. [expected mp 100.5-101.5° C.]; ¹H NMR: δ 2.56 (t, 4H, J=7.4 Hz), 2.74 (t, 4H, J=6.8 Hz), 3.29 (s, 4H), 7.04 (m, 20H); ¹³C NMR: δ 29.6, 37.3, 42.5, 71.1, 126.1, 126.8, 128.4, 129.6, 136.0, 140.6, 207.8; Anal. Calcd for C₃₃H₃₂O₂: C, 86.05; H, 7.00. Found: C, 86.28; H, 7.11. 1,5-Bis(4-hydroxy-3-methoxyphenyl)-1,4-pentadien-3-one (20a). 1,5-Bis(4-methoxymethoxy-3-methoxyphenyl)-1,4-pentadien-3-one (20j, 0.41 g, 10.0 mmol) was stirred in methanol (50 ml) for 15 min at 50° C. Concentrated hydrochloric acid (1 drop) was added and the solution stirred for 3 hr at 50° C. The methanol was evaporated and the resulting residue extracted into ethyl acetate, washed with saturated sodium chloride, dried over magnesium sulfate, filtered and evaporated to afford a semi-solid. The crude semi-solid was purified by preparative thin layer chromatography with ethyl acetate/hexane to give 0.31 g (96%) of a yellow solid: mp 84-86° C. [expected mp 82-83° C.]; ¹H NMR: δ 3.89 (s, 6H), 6.87 (d, 2H, J=8.4 Hz), 6.88 (d, 2H, J=15.9 Hz), 7.10 (m, 4H), 7.62 (d, 2H, J=15.9 Hz); ¹³C NMR: δ 56.1, 109.8, 114.8, 123.3, 123.4, 127.5, 143.0, 146.8, 148.1, 188.6.

1,5-Diphenyl-1,4-pentadien-3-one (20b). Benzaldehyde (1b, 2.54 ml, 25.0 mmol) and acetone (19, 0.90 ml, 12.2 mmol) were combined in ethanol (20 ml) and stirred for 15 min at room temperature. A solution of sodium hydroxide (2.50 g, 62.5 mmol) and water (25 ml) was added and the solution stirred for 3 hr at room temperature. The resulting precipitate was filtered and recrystallized from ethanol to afford 2.35 g (82%) of yellow crystals: mp 110-112° C. [expected mp 112-114° C.]; ¹H NMR: δ 7.07 (d, 2H, J=15.9 Hz), 7.40 (m, 8H), 7.61 (m, 2H), 7.73 (d, 2H, J=15.9 Hz); ¹³C NMR: δ 125.4, 128.3, 12.9, 130.4, 134.7, 143.2, 188.7.

1,5-Bis(2-methoxyphenyl)-1,4-pentadien-3-one (20c). 2-Methoxybenzaldehyde (1c, 1.50 ml, 12.4 mmol) and acetone (19,0.46 ml, 6.2 mmol) were combined in ethanol (10 ml) and stirred for 15 min at room temperature. A solution of sodium hydroxide (0.50 g, 12.5 mmol) and water (10 ml) was added and the mixture stirred for 18 hr at room temperature. The resulting precipitate was filtered and recrystallized from ethanol to give 1.56 g (85%) of a yellow solid: mp 123-124° C. [expected mp 124° C.]; ¹H NMR: δ 3.87 (s, 6H), 6.91 (m, 4H), 7.15 (d, 2H, J=16.1 Hz), 7.33 (dt, 2H, J=7.2, 1.4 Hz), 7.59 (d, 2H, J=7.5 Hz), 8.06 (d, 2H, J=16.3 Hz); ¹³C NMR: δ 55.4, 111.1, 120.6, 123.8, 126.1, 128.5, 131.4, 138.0, 158.4, 189.6.

1,5-Bis(2,3-dimethoxyphenyl)-1,4-pentadien-3-one (20d). 2,3-Dimethoxy-benzaldehyde (1d, 4.50 g, 27.1 mmol) and acetone (19, 1.00 ml, 13.5 mmol) were combined in ethanol (25 ml) and stirred for 15 min at room temperature. A solution of sodium hydroxide (2.20 g, 55.0 mmol) and water (25 ml) was added and the mixture stirred for 6 hr at room temperature. The resulting precipitate was filtered and recrystallized from ethanol to give 4.15 g (87%) of a yellow solid: mp 106-108° C. [expected mp 108° C.]; ¹H NMR: δ 3.89 (s, 6H), 3.90 (s, 6H), 6.96 (d, 2H, J=8.1 Hz), 7.09 (t, 2H, J=8.1 Hz), 7.16 (d, 2H, J=16.3 Hz), 7.25 (d, 2H, J=7.4 Hz), 8.05 (d, 2H, J=16.1 Hz); ¹³C NMR: δ 55.9, 61.3, 114.1, 119.3, 124.1, 126.8, 129.0, 137.8, 148.7, 153.0, 189.5.

1,5-Bis(4-methoxyphenyl)-1,4-pentadien-3-one (20e). 4-Methoxybenzaldehyde (1e, 1.50 ml, 12.3 mmol) and acetone (19,0.45 ml, 6.2 mmol) were combined in ethanol (20 ml) and stirred for 15 min at room temperature. A solution of sodium hydroxide (2.53 g, 63.3 mmol) and water (25 ml) was added and the mixture stirred for 3 hr at room temperature. The resulting precipitate was filtered and recrystallized from ethanol to give 1.00 g (55%) of a yellow solid: mp 128-130° C. [expected mp 133-134° C.]; ¹H NMR: 3.82 (s, 6H), 6.90 (d, 4H, J=8.5 Hz), 6.93 (d, 2H, J=15.9 Hz), 7.54 (d, 4H, J=8.5 Hz), 7.68 (d, 2H, J=15.9 Hz); ¹³C NMR: δ 55.4, 114.4, 123.5, 127.6, 129.9, 142.5, 161.4, 188.6.

1,5-Bis(4-hydroxyphenyl)-1,4-pentadien-3-one (20f). 4-Hydroxybenzaldehyde (1f, 2.00 g, 16.4 mmol) and acetone (19, 0.61 ml, 8.3 mmol) were combined in ethanol (30 ml) and stirred for 15 min at room temperature. A solution of sodium hydroxide (1.00 g, 25.0 mmol) and water (30 ml) was added and the mixture stirred for 4 hr at room temperature. The resulting mixture was extracted into ethyl acetate, washed with saturated sodium chloride, dried over magnesium sulfate, filtered and evaporated to afford a solid. The crude solid was recrystallized from ethyl acetate/hexane to give 0.85 g (39%) of a yellow solid: mp 235-237° C. [expected mp 238-239° C.]; ¹H NMR: (DMSO) δ 6.82 (d, 4H, J=8.5 Hz), 7.08 (d, 2H, J=16.1 Hz), 7.61 (d, 4H, J=8.5 Hz), 7.64 (d, 2H, J=15.7 Hz), 10.08 (s, 2H); ¹³C NMR: (DMSO) δ 115.8, 122.6, 125.7, 130.3, 142.3, 159.7, 187.9.

1,5-Bis(4-dimethylaminophenyl)-1,4-pentadien-3-one (20 g). 4-Dimethylamino-benzaldehyde (1 g, 1.00 g, 6.7 mmol) and acetone (19, 0.24 ml, 3.2 mmol) were combined in ethanol (10 ml) and stirred for 15 min at room temperature. A solution of sodium hydroxide (0.40 g, 10 mmol) and water (10 ml) was added and the mixture stirred for 18 hr at room temperature. The resulting mixture was extracted into ethyl acetate, washed with saturated sodium chloride, dried over magnesium sulfate, filtered and evaporated to afford a solid. The crude solid was recrystallized from ethanol to give 0.53 g (51%) of an orange solid: mp 179-181° C. [expected mp 174-176° C.]; ¹H NMR: δ 3.01 (s, 12H), 6.69 (d, 4H, J=8.7 Hz), 6.87 (d, 2H, J=15.7 Hz), 7.50 (d, 4H, J=8.7 Hz), 7.67 (d, 2H, J=15.7 Hz); ¹³C NMR: δ 40.2, 98.9, 111.8, 121.2, 122.9, 129.9, 142.8, 151.6.

1,5-Bis(3,4-dimethoxyphenyl)-1,4-pentadien-3-one (20i). 3,4-Dimethoxy-benzaldehyde (1i, 2.25 g, 13.5 mmol) and acetone (19, 0.50 ml, 6.8 mmol) were combined in ethanol (15 ml) and stirred for 15 min at room temperature. A solution of sodium hydroxide (1.10 g, 27.5 mmol) and water (10 ml) was added and the mixture stirred for 2 hr at room temperature. The resulting precipitate was filtered and recrystallized from ethanol to give 1.80 g (75%) of a yellow solid: mp 72-75° C. [expected mp 68-70° C.]; ¹H NMR: δ 3.92 (s, 6H), 3.94 (s, 6H), 6.89 (d, 2H, J=8.3 Hz), 6.96 (d, 2H, J=15.9 Hz), 7.14 (s, 2H), 7.20 (d, 2H, J=8.1 Hz), 7.69 (d, 2H, J=15.9 Hz); ¹³C NMR: δ 55.9, 109.9, 111.0, 122.9, 123.5, 127.7, 142.8, 149.1, 151.2, 188.4.

1,5-Bis(4-methoxymethyloxy-3-methoxyphenyl)-1,4-pentadien-3-one (20j). 4-Methoxymethyloxy-3-methoxybenzaldehyde (1j, 1.95 g, 10.0 mmol) and acetone (19, 0.37 ml, 5.0 mmol) were combined in ethanol (25 ml) and stirred for 15 min at room temperature. A solution of sodium hydroxide (0.65 g, 16.3 mmol) and water (25 ml) was added and the solution stirred for 18 hr at room temperature. The resulting mixture was extracted into dichloromethane, washed with saturated sodium chloride, dried over magnesium sulfate, filtered and evaporated to afford an oil. The crude oil was chromatographed on silica gel with ethyl acetate/hexane to give 1.40 g (67%) of a yellow solid: mp 81-82° C.; ¹H NMR: δ 3.53 (s, 6H), 3.95 (s, 6H), 5.29 (s, 4H), 6.97 (d, 2H, J=15.9 Hz), 7.17 (m, 6H), 7.69 (d, 2H, J=15.9 Hz); ¹³C NMR: δ 56.0, 56.4, 95.2, 110.8, 115.9, 122.5, 124.0, 124.6, 129.2, 142.8, 148.7, 149.8, 188.5.

1,5-Bis(3-methoxyphenyl)-1,4-pentadien-3-one (20k). 3-Methoxybenzaldehyde (1k, 3.09 ml, 25.4 mmol) and acetone (19, 0.94 ml, 12.7 mmol) were combined in ethanol (20 ml) and stirred for 15 min at room temperature. A solution of sodium hydroxide (1.50 g, 37.5 mmol) and water (20 ml) was added and the mixture stirred for 18 hr at room temperature. The resulting mixture was extracted into ethyl acetate, washed with saturated sodium chloride, dried over magnesium sulfate, filtered and evaporated to afford an oil. The crude oil was chromatographed on silica gel with ethyl acetate/hexane to yield a solid. The solid was recrystallized from ethanol to give 2.06 g (62%) of a yellow solid: mp 64-65° C. [expected mp 52-54° C.]; ¹H NMR: δ 3.83 (s, 6H), 6.94 (dd, 2H, J=8.1, 2.4 Hz), 7.04 (d, 2H, J=15.9 Hz), 7.15 (m, 4H), 7.32 (t, 2H, J=8.0 Hz), 7.68 (d, 2H, J=16.1 Hz); ¹³C NMR; δ 55.2, 113.2, 116.2, 120.9, 125.5, 129.8, 136.0, 143.0, 159.8, 188.6.

1,5-Bis(2,6-dimethoxyphenyl)-1,4-pentadien-3-one (20l). 2,6-Dimethoxy-benzaldehyde (20l, 1.00 g, 6.0 mmol) and acetone (19, 0.44 ml, 3.0 mmol) were combined in ethanol (10 ml) and stirred for 10 min at room temperature. A solution of sodium hydroxide (0.72 g, 9.0 mmol) and water (15 ml) was added and the mixture stirred for 18 hr at room temperature. The resulting precipitate was filtered and recrystallized from ethanol to give 0.66 g (63%) of a yellow solid: mp 152-154° C. [expected mp 152-154° C.]; ¹H NMR: δ 3.90 (s, 12H), 6.57 (d, 4H, J=8.5 Hz), 7.26 (t, 2H, J=8.5 Hz), 7.59 (d, 2H, J=16.3 Hz), 8.17 (d, 2H, J=16.3 Hz); ¹³C NMR: δ 55.8, 103.7, 113.1, 129.0, 130.9, 133.3, 160.0, 192.4.

1,5-Bis(2,5-dimethoxyphenyl)-1,4-pentadien-3-one (20m). 2,5-Dimethoxy-benzaldehyde (1m, 2.00 g, 12.0 mmol) and acetone (19, 0.44 ml, 6.0 mmol) were combined in ethanol (15 ml) and stirred for 15 min at room temperature. A solution of sodium hydroxide (0.72 g, 18.0 mmol) and water (15 ml) was added and the mixture stirred for 18 hr at room temperature. The resulting precipitate was filtered and recrystallized from ethanol to give 1.46 g (69%) of a yellow solid: mp 105-106° C. [expected mp 105-106° C.]; ¹H NMR: δ 3.79 (s, 6H), 3.85 (s, 6H), 6.88 (m, 4H), 7.11 (d, 2H, J=2.8 Hz), 7.12 (d, 2H, J=16.1 Hz), 8.01 (d, 2H, J=16.1 Hz); ¹³C NMR: δ 55.8, 56.1, 112.4, 113.1, 117.1, 124.5, 126.3, 137.9, 153.0, 153.4, 189.6.

1,5-Bis(2,4-dimethoxyphenyl)-1,4-pentadien-3-one (20n). 2,4-Dimethoxy-benzaldehyde (1n, 2.00 g, 12.0 mmol) and acetone (19, 0.44 ml, 6.0 mmol) were combined in ethanol (15 ml) and stirred for 15 min at room temperature. A solution of sodium hydroxide (0.72 g, 18.0 mmol) and water (15 ml) was added and the mixture stirred for 18 hr at room temperature. The resulting precipitate was filtered and recrystallized from ethanol to give 1.71 g (81%) of a yellow solid: mp 138-140° C. [expected mp 138-139° C.]; ¹H NMR: δ 3.81 (s, 6H), 3.85 (s, 6H), 6.43 (d, 2H, J=2.2 Hz), 6.48 (dd, 2H, J=8.5, 2.2 Hz), 7.04 (d, 2H, J=16.1 Hz), 7.52 (d, 2H, J=8.5 Hz), 7.96 (d, 2H, J=16.1 Hz); ¹³C NMR: δ 55.4, 55.5, 98.3, 105.3, 117.1, 124.1, 130.0, 137.6, 159.9, 162.6, 189.7.

1,5-Bis(3,5-dimethoxyphenyl)-1,4-pentadien-3-one (20o). 3,5-Dimethoxy-benzaldehyde (1o, 2.00 g, 12.0 mmol) and acetone (19, 0.44 ml, 6.0 mmol) were combined in ethanol (15 ml) and stirred for 15 min at room temperature. A solution of sodium hydroxide (0.72 g, 18.0 mmol) and water (15 ml) was added and the mixture stirred for 18 hr at room temperature. The resulting precipitate was filtered and recrystallized from ethanol to give 1.32 g (63%) of a yellow solid: mp 126-128° C. [expected mp 124.5-125.5° C.]; ¹H NMR: δ 3.80 (s, 12H), 6.49 (s, 2H), 6.73 (d, 4H, J=2.0 Hz), 7.00 (d, 2H, J=15.9 Hz), 7.62 (d, 2H, J=15.7 Hz); ¹³C NMR: δ 55.4, 102.7, 106.2, 125.7, 136.8, 143.2, 160.9, 188.6.

1,5-Bis(3-hydroxyphenyl)-1,4-pentadien-3-one (20p). 3-Hydroxybenzaldehyde (1p, 2.07 g, 17.0) and acetone (19, 0.62 ml, 8.4 mmol) were combined in ethanol (15 ml) and stirred for 15 min at room temperature. A solution of sodium hydroxide (1.00 g, 25.0 mmol) and water (4 ml) was added and the solution stirred for 48 hr at room temperature. The resulting mixture was neutralized with hydrochloric acid (1 N), extracted with ethyl acetate, washed with saturated sodium chloride, dried over magnesium sulfate, filtered and evaporated to afford a solid. The crude solid was recrystallized from ethyl acetate to give 0.42 g (19%) of a brown solid: mp 190-195° C. [expected mp 198-200° C.]; ¹H NMR: (DMSO) δ 6.83 (d, 2H, J=7.0 Hz), 7.22 (m, 8H), 7.68 (d, 2H, J=16.1 Hz), 9.63 (s, 2H); ¹³C NMR: (DMSO) δ 114.7, 117.5, 119.4, 125.4, 129.7, 135.8, 142.7, 157.5, 168.2.

1,5-Bis(2-hydroxyphenyl)-1,4-pentadien-3-one (20q). 2-Hydroxybenzaldehyde (1q, 1.81 ml, 17.0 mmol) and acetone (19, 0.62 ml, 8.4 mmol) were combined in ethanol (15 ml) and stirred for 15 min at room temperature. A solution of sodium hydroxide (1.00 g, 25.0 mmol) and water (4 ml) was added and the solution stirred for 1 week at room temperature. The mixture was neutralized with hydrochloric acid (1 N) and the resulting precipitate filtered and recrystallized from ethyl acetate/hexane to give 1.79 g (80%) of a yellow solid: mp 154-157° C. [expected mp 155° C.]; ¹H NMR: (DMSO) δ 6.89 (m, 4H), 7.27 (m, 4H), 7.68 (d, 2H, J=7.4 Hz), 7.93 (d, 2H, J=16.1 Hz), 10.22 (s, 2H); ¹³C NMR: (DMSO) δ 116.1, 119.3, 121.3, 125.3, 128.5, 131.5, 137.6, 156.9, 188.5.

1,5-Bis(4-fluorophenyl)-1,4-pentadien-3-one (20r). 4-Fluorobenzaldehyde (1r, 0.75 ml, 7.0 mmol) and acetone (19, 0.26 ml, 3.5 mmol) were combined in ethanol (30 ml) and stirred for 10 min at room temperature. A solution of sodium hydroxide (0.50 g, 12.5 mmol) and water (20 ml) was added and the mixture stirred for 18 hr at room temperature. The resulting precipitate was filtered and recrystallized from ethanol to afford 0.82 g (86%) of a yellow solid: mp 150-152° C. [expected mp 152-154° C.]; ¹H NMR: δ 6.97 (d, 2H, J=15.9 Hz), 7.09 (m, 4H), 7.58 (m, 4H), 7.68 (d, 2H, J=15.9 Hz); ¹³C NMR: δ 116.1, 1125.1, 130.2, 130.9, 142.0, 164.0, 188.3.

1,5-Bis(3-fluorophenyl)-1,4-pentadien-3-one (20s). 3-Fluorobenzaldehyde (1s, 0.5 ml, 4.7 mmol) and acetone (19, 0.18 ml, 2.3 mmol) were combined in ethanol (20 ml) and stirred for 10 min at room temperature. A solution of sodium hydroxide (0.29 g, 7.3 mmol) and water (15 ml) was added and the mixture stirred for 18 hr at room temperature. The resulting mixture was filtered and chromatographed on silica gel with ethyl acetate/hexane to give a solid. The crude was recrystallized from ethanol to afford 0.26 g (42%) of yellow crystals: mp 96-97° C. [expected mp 96-97° C.]; ¹H NMR: δ 7.03 (d, 2H, J=16.1 Hz), 7.09 (m, 2H), 7.35 (m, 6H), 7.67 (d, 2H, J=15.9 Hz); ¹³C NMR: δ 114.4, 117.5, 124.4, 126.3, 130.4, 136.9, 142.0, 162.9, 188.1; Anal. Calcd for C₁₇H₁₂OF₂: C, 75.55; H, 4.48. Found: C, 75.26; H, 4.65.

1,5-Bis(2-fluorophenyl)-1,4-pentadien-3-one (20t). 2-Fluorobenzaldehyde (1t, 0.5 ml, 4.7 mmol) and acetone (19, 0.18 ml, 2.4 mmol) were combined in ethanol (20 ml) and stirred for 10 min at room temperature. A solution of sodium hydroxide (0.29 g, 7.3 mmol) and water (15 ml) was added and the mixture stirred for 18 hr at room temperature. The resulting mixture was filtered and chromatographed on silica gel with ethyl acetate/hexane to give a solid. The crude solid was recrystallized from ethanol to afford 0.27 g (41%) of yellow crystals: mp 68-72° C. [expected mp 68-70° ]; ¹H NMR: δ 7.13 (m, 6H), 7.36 (m, 2H), 7.61 (dt, 2H, J=7.6 Hz, 1.4 Hz), 7.84 (d, 2H, J=16.3 Hz); ¹³C NMR: δ 116.2, 122.8, 124.4, 127.6, 129.3, 131.8, 135.9, 161.5, 188.7.

1,5-Bis(4-trifluoromethyl)-1,4-pentadien-3-one (20u). 4-(Trifluoromethyl)-benzaldehyde (1u, 0.50 ml, 3.7 mmol) and acetone (19, 0.13 ml, 1.8 mmol) were combined in ethanol (15 ml) and stirred for 10 min at room temperature. A solution of sodium hydroxide (0.22 g, 5.5 mmol) and water (15 ml) was added and the mixture stirred for 18 hr at room temperature. The resulting precipitate was filtered and recrystallized from ethanol to afford 0.57 g (87%) of a yellow solid: mp 151-154° C. [expected mp 156-157° C.]; ¹H NMR: δ 7.12 (d, 2H, J=15.9 Hz), 7.69 (m, 8H), 7.73 (d, 2H, J=15.9 Hz); ¹³C NMR: δ 121.6, 125.9, 127.2, 128.5, 132.1, 138.0, 141.8, 187.9.

1,5-Bis(3-trifluoromethyl)-1,4-pentadien-3-one (20v). 3-(Trifluoromethyl)-benzaldehyde (1v, 0.5 ml, 3.7 mmol) and acetone (19, 0.14 ml, 1.9 mmol) were combined in ethanol (15 ml) and stirred for 10 min at room temperature. A solution of sodium hydroxide (0.23 g, 5.8 mmol) and water (15 ml) was added and the mixture stirred for 18 hr at room temperature. The resulting mixture was filtered and chromatographed on silica gel with ethyl acetate/hexane to give a solid. The crude solid was recrystallized from ethanol to give 0.25 g (36%) of yellow crystals: mp 116-117° C. [expected mp 116-117° C.]; ¹H NMR: δ 7.12 (d, 2H, J=15.9 Hz) 7.53 (t, 2H, J=7.6 Hz), 7.66 (d, 2H, J=8.0 Hz), 7.73 (d, 2H, J=7.2 Hz), 7.82 (m, 4H); ¹³C NMR: δ 123.7, 124.7, 126.7, 126.8, 129.5, 131.5, 131.6, 135.4, 141.8, 187.8; Anal. Calcd for C₁₉H₁₂OF₆: C, 61.63; H, 3.27. Found: C, 61.82; H, 3.28.

1,5-Bis(2-trifluoromethyl)-1,4-pentadien-3-one (20w). 2-(Trifluoromethyl)-benzaldehyde (1w, 0.75 ml, 5.7 mmol) and acetone (19, 0.21 ml, 2.8 mmol) were combined in ethanol (20 ml) and stirred for 10 min at room temperature. A solution of sodium hydroxide (0.34 g, 8.5 mmol) and water (15 ml) was added and the mixture stirred for 18 hr at room temperature. The resulting precipitate was filtered and recrystallized from ethanol to afford 0.92 g (87%) of a yellow solid: mp 131-133° C. [expected mp 131° C.]; ¹H NMR: δ 6.99 (d, 2H, J=15.9 Hz), 7.48 (t, 2H, J=7.6 Hz), 7.56 (t, 2H, J=7.0 Hz), 7.70 (d, 2H, J=7.70 (d, 2H, J=7.7 Hz), 7.77 (d, 2H, J=7.6 Hz), 8.07 (d, 2H, J=15.7 Hz); ¹³C NMR: δ 123.9, 126.2, 127.9, 128.8, 129.4, 129.7, 132.1, 133.7, 139.1, 188.0.

1,5-Bis(4-chlorophenyl)-1,4-pentadien-3-one (20x). 4-Chlorobenzaldehyde (1x, 1.00 g, 7.1 mmol) and acetone (19, 0.26 ml, 3.5 mmol) were combined in ethanol (10 ml) and stirred for 15 min at room temperature. A solution of sodium hydroxide (0.40 g, 10.0 mmol) and water (10 ml) was added and the mixture stirred for 3 hr at room temperature. The resulting precipitate was filtered and recrystallized from ethyl acetate to give 0.75 g (70%) of yellow crystals: mp 187-189° C. [expected mp 191-193° C.]; ¹H NMR: δ 7.00 (d, 2H, J=15.9 Hz), 7.37 (d, 4H, J=8.5 Hz), 7.52 (d, 4H, J=8.5 Hz), 7.66 (d, 2H, J=15.9 Hz); ¹³C NMR: δ 125.7, 129.2, 129.5, 133.2, 136.4, 141.9, 188.1.

1,5-Bis(3-chlorophenyl)-1,4-pentadien-3-one (20y). 3-Chlorobenzaldehyde (1y, 2.00 ml, 17.7 mmol) and acetone (19, 0.65 ml, 8.8 mmol) were combined in ethanol (20 ml) and stirred for 15 min at room temperature. A solution of sodium hydroxide (1.00 g, 25.0 mmol) and water (20 ml) was added and the mixture stirred for 2 hr at room temperature. The resulting precipitate was filtered and recrystallized from ethyl acetate to give 2.41 g (90%) of a yellow solid: mp 125-127° C. [expected mp 120-121° C.]; ¹H NMR: δ 7.03 (d, 2H, J=15.9 Hz), 7.33 (m, 4H), 7.45 (d, 2H, J=6.6 Hz), 7.58 (m, 2H), 7.64 (d, 2H, J=15.9 Hz); ¹³C NMR: δ 126.3, 126.6, 127.9, 130.1, 130.3, 134.9, 136.5, 141.8, 188.0.

1,5-Bis(2-chlorophenyl)-1,4-pentadien-3-one (20z). 2-Chlorobenzaldehyde (1z, 2.00 ml, 17.8 mmol) and acetone (19, 0.65 ml, 8.8 mmol) were combined in ethanol (20 ml) and stirred for 15 min at room temperature. A solution of sodium hydroxide (1.00 g, 25.0 mmol) and water (20 ml) was added and the mixture stirred for 18 hr at room temperature. The resulting precipitate was filtered and recrystallized from ethyl acetate to give 1.80 g (67%) of a yellow solid: mp 119-121° C. [expected mp 110° C.]; ¹H NMR: δ 7.04 (d, 2H, J=16.1 Hz), 7.29 (m, 4H), 7.41 (m, 2H), 7.67 (m, 2H), 8.11 (d, 2H, J=16.1 Hz); ¹³C NMR: δ 127.0, 127.5, 127.6, 130.1, 131.1, 132.9, 135.3, 139.2, 188.4.

1,5-Bis(4-methylphenyl)-1,4-pentadien-3-one (20aa). 4-Methylbenzaldehyde (1aa, 1.50 ml, 12.7 mmol) and acetone (19, 0.47 ml, 6.4 mmol) were combined in ethanol (10 ml) and stirred for 15 min at room temperature. A solution of sodium hydroxide (0.52 g, 13.0 mmol) and water (10 ml) was added and the mixture stirred for 1 hr at room temperature. The resulting precipitate was filtered and recrystallized from ethanol to give 1.30 g (78%) of a yellow solid: mp 174-176° C. [expected mp 171-172° C.]; ¹H NMR: δ 2.37 (s, 6H), 7.02 (d, 2H, J=15.9 Hz), 7.20 (d, 4H, J=8.0 Hz), 7.50 (d, 4H, J=7.9 Hz), 7.70 (d, 2H, J=15.9 Hz); ¹³C NMR: δ 21.6, 124.6, 128.3, 129.6, 132.1, 140.8, 143.0, 188.9.

1,5-Bis(3-methylphenyl)-1,4-pentadien-3-one (20ab). 3-Methylbenzaldehyde (1ab, 3.00 ml, 25.4 mmol) and acetone (19, 0.94 ml, 12.7 mmol) were combined in ethanol (20 ml) and stirred for 15 min at room temperature. A solution of sodium hydroxide (1.50 g, 37.5 mmol) and water (20 ml) was added and the mixture stirred for 18 hr at room temperature. The resulting mixture was extracted into ethyl acetate, washed with saturated sodium chloride, dried over magnesium sulfate, filtered and evaporated to afford a solid. The crude solid was recrystallized from ethanol to give 2.39 g (72%) of a yellow solid: mp 68-72° C. [expected mp 68-72° C.]; ¹H NMR: δ 2.38 (s, 6H), 7.06 (d, 2H, J=15.9 Hz), 7.26 (m, 4H), 7.40 (m, 4H), 7.70 (d, 2H, J=15.9 Hz); ¹³C NMR: δ 21.3, 125.1, 125.4, 128.6, 128.8, 131.1, 134.6, 138.4, 143.1, 188.6.

1,5-Bis(2-methylphenyl)-1,4-pentadien-3-one (20ac). 2-Methylbenzaldehyde (1ac, 1.45 ml, 12.5 mmol) and acetone (19, 0.46 ml, 6.3 mmol) were combined in ethanol (20 ml) and stirred for 15 min at room temperature. A solution of sodium hydroxide (2.61 g, 65.3 mmol) and water (25 ml) was added and the mixture stirred for 3 hr at room temperature. The resulting mixture was extracted into ethyl acetate, washed with saturated sodium chloride, dried over magnesium sulfate, filtered and evaporated to afford a solid. The crude solid was recrystallized from ethanol to give 0.71 g (43%) of a yellow solid: mp 98-100° C. [expected mp 94-96° C.]; ¹H NMR: δ 2.47 (s, 6H), 6.98 (d, 2H, J=15.9 Hz), 7.24 (m, 6H), 7.64 (d, 2H, J=7.2 Hz), 8.03 (d, 2H, J=15.9 Hz); ¹³C NMR: δ 19.7, 126.1, 126.2, 126.5, 129.9, 130.7, 133.6, 137.9, 140.5, 188.5.

1,5-Bis(4-carbmethoxyphenyl)-1,4-pentadien-3-one (20ae). 4-Carbmethoxy-benzaldehyde (1ae, 0.62 g, 3.8 mmol) and acetone (19, 0.14 ml, 1.9 mmol) were combined in methanol (20 ml) and stirred under a nitrogen atmosphere for 15 min at room temperature. A solution of sodium hydroxide (0.15 g, 3.8 mmol) in water (5 ml) was added the mixture stirred for 18 hr at room temperature under a nitrogen atmosphere. The resulting precipitate was filtered and recrystallized from xylene to give 0.29 g (44%) of a yellow solid: mp 206-210° C. [expected mp 221-223° C.]; ¹H NMR: δ 3.92 (s, 6H), 7.12 (d, 2H, J=16.1 Hz), 7.65 (d, 4H, J=8.1 Hz), 7.73 (d, 2H, J=15.9 Hz), 8.06 (d, 4H, J=8.0 Hz); ¹³C NMR: δ 52.3, 127.1, 128.1, 130.1, 131.6, 138.8, 142.1, 166.2, 188.0.

1,5-Bis(3,4-dihydroxyphenyl)-1,4-pentadien-3-one (20af). 1,5-Bis(3,4-dimeth-oxyphenyl)-1,4-pentadien-3-one (20i, 0.56 g, 1.6 mmol) was dissolved in dichloromethane (10 ml) and stirred under a nitrogen atmosphere at -78° C. for 5 min. Boron tribromide (0.90 ml, 9.5 mmol) was added and stirring continued for 60 min at −78° C., 60 min at 0° C. and 60 min at room temperature. The mixture was poured into hydrochloric acid (30 ml, 1 N) and stirring was continued for 18 hr at room temperature. The resulting mixture was extracted into ethyl acetate, washed with water and saturated sodium chloride, dried over magnesium sulfate, filtered and evaporated to afford a solid. The crude solid was chromatographed on silica gel with ethyl acetate/hexane to give 0.36 g (76%) of an orange solid: mp >250° C. [expected mp 221-223° C.]; ¹H NMR: (DMSO) δ 6.78 (d, 2H, J=8.1 Hz), 6.99 (d, 2H, J=15.9 Hz), 7.06 (d, 2H, J=7.9 Hz), 7.14 (s, 2H), 7.55 (d, 2H, J=15.7 Hz), 9.15 (s, 2H), 9.63 (s, 2H); ¹³C NMR: (DMSO) δ 114.9, 115.6, 121.5, 122.5, 126.2, 142.5, 145.4, 148.2, 187.6.

1,5-Bis(4-acetoxy-3-methoxyphenyl)-1,4-pentadien-3-one (20ag). 1,5-Bis(4-hydroxy-3-methoxyphenyl)-1,4-pentadien-3-one (20a, 0.32 g, 1.0 mmol) was dissolved in acetic anhydride (21, 7.00 ml, 74.1 mmol) and stirred for 5 min at room temperature. Pyridine (0.70 ml, 8.7 mmol) was added and the mixture stirred for 30 min at 100° C. The resulting mixture was poured into water, extracted with ethyl acetate, washed with saturated sodium chloride, dried over magnesium sulfate, filtered and evaporated to afford a solid. The crude solid was recrystallized from tetrahydrofuran/hexane to give 0.36 g (88%) of a yellow solid: mp 179-180° C. [expected mp 150° C.]; ¹H NMR: δ 2.31 (s, 6H), 3.87 (s, 6H), 6.98 (d, 2H, J=15.9 Hz), 7.06 (d, 2H, J=8.1 Hz), 7.18 (m, 4H), 7.67 (d, 2H, J=15.9 Hz); ¹³C NMR: δ 20.7, 56.0, 111.7, 121.4, 123.3, 125.5, 133.7, 141.6, 142.6, 151.4, 168.5, 188.3; Exact mass calcd for C₂₃H₂₂O₇: 410.1366, observed (M+H) 411.1444.

1,5-Bis(4-acetoxyphenyl)-1,4-pentadien-3-one (20ah). 1,5-Bis(4-hydroxy-phenyl)-1,4-pentadien-3-one (20f, 0.26 g, 1.0 mmol) was dissolved in acetic anhydride (21, 7.00 ml, 74.1 mmol) and stirred for 5 min at room temperature. Pyridine (0.70 ml, 8.7 mmol) was added and the mixture stirred for 30 min at 100 ° C. The resulting mixture was poured into water, extracted with ethyl acetate, washed with saturated sodium chloride, dried over magnesium sulfate, filtered and evaporated to afford a solid. The crude solid was recrystallized from tetrahydrofuran/hexane to give 0.28 g (82%) of a yellow solid: mp 167-168° C. [expected mp 167-168° C.]; ¹H NMR: δ 2.30 (s, 6H), 7.00 (d, 2H, J=15.9 Hz), 7.13 (d, 4H, J=8.3 Hz), 7.60 (d, 4H, J=8.2 Hz), 7.69 (d, 2H, J=15.9 Hz); ¹³C NMR: δ 21.1, 122.1, 125.4, 129.4, 132.4, 142.1, 152.2, 168.9, 188.3; Exact mass calcd for C₂₁H₁₈O₅: 350.1154, observed (M+H) 351.1232.

1,5-Bis(l-naphthyl)-1,4-pentadien-3-one (23). 1-Naphthaldehyde (22, 1.36 ml, 10.0 mmol) and acetone (19, 0.37 ml, 5.0 mmol) were combined in ethanol (10 ml) and stirred for 15 min at room temperature. A solution of sodium hydroxide (0.40 g, 10.0 mmol) and water (10 ml) was added and the mixture stirred for 18 hr at room temperature. The resulting mixture was extracted into ethyl acetate, washed with saturated sodium chloride, filtered and evaporated to afford a solid. The crude solid was recrystallized from ethyl acetate to give 0.63 g (38%) of a yellow solid: mp 132-133° C. [expected mp 128° C.]; ¹H NMR: δ 7.24 (d, 2H, J=15.7 Hz), 7.57 (m, 6H), 7.92 (m, 6H), 8.28 (d, 2H, J=8.0 Hz), 8.65 (d, 2H, J=15.5 Hz); ¹³C NMR: δ 123.4, 125.1, 125.4, 126.2, 126.9, 128.1, 128.7, 130.7, 131.7, 132.2, 133.7, 140.3, 188.5.

1,5-Bis(2-naphthyl)-1,4-pentadien-3-one (25). 2-Naphthaldehyde (24, 1.56 g, 10.0 mmol) and acetone (19, 0.37 ml, 5.0 mmol) were combined in ethanol (10 ml) and stirred for 15 min at room temperature. A solution of sodium hydroxide (0.60 g, 15.0 mmol) and water (10 ml) was added and the mixture stirred for 18 hr at room temperature. The resulting precipitate was filtered and recrystallized from ethanol to give 1.16 g (69%) of a yellow solid: mp 244-246° C. [expected mp 243-244° C.]; ¹H NMR: 6 7.23 (d, 2H, J=15.9 Hz), 7.52 (m, 4H), 7.83 (m, 8H), 7.93 (d, 2H, J=15.9 Hz), 8.03 (s, 2H); ¹³C NMR: δ 123.6, 125.7, 126.7, 127.3, 127.8, 128.6, 128.7, 130.5, 132.3, 133.3, 134.3, 143.1, 190.0.

1,5-Bis(4-pyridinium chloride)-1,4-pentadien-3-one (28). 1,3-Acetone-dicarboxylic acid (27, 1.05 g, 7.2 mmol) was dissolved in ethanol (10 ml) and stirred for 15 min at room temperature. 4-Pyridinecarboxaldehyde (26, 1.37 ml, 14.4 mmol) was added dropwise and the mixture stirred for 2 hr at room temperature. Hydrochloric acid (5 ml) was added and the mixture stirred for 1 hr at 80° C. The resulting precipitate was filtered and recrystallized from water/acetone to give 0.59 g (27%) of a yellow solid: mp 247-249° C. [expected mp 247-249° C.]; ¹H NMR: (D₂O) δ 7.54 (d, 2H, J=16.3 Hz), 7.78 (d, 2H, J=15.9 Hz), 8.15 (d, 4H, J=6.6 Hz), 8.70 (d, 4H, J=6.6 Hz); ¹³C NMR: (D₂O) δ 128.2, 136.2, 141.3, 144.2, 154.4, 193.0.

1,5-Bis(4-pyridyl)-1,4-pentadien-3-one (29). 1,5-Bis(4-pyridinium chloride)-1,4-pentadien-3-one (28, 0.25 g, 0.8 mmol) and sodium hydroxide (0.80 g, 20 mmol) were combined in water (20 ml) and stirred for 15 min at room temperature. The resulting mixture was extracted with ethyl acetate, washed with saturated sodium chloride, dried over magnesium sulfate, filtered and evaporated to afford a solid. The crude solid was recrystallized from ethyl acetate/hexane to give 0.16 g (84%) of a yellow solid: mp 145-146° C. [expected mp 149° C.]; ¹H NMR: δ 7.17 (d, 2H, J=15.9 Hz), 7.42 (d, 4H, J=5.6 Hz), 7.63 (d, 2H, J=15.9 Hz), 8.67 (d, 4H, J=5.6 Hz); ¹³C NMR: δ 121.9, 128.6, 141.0, 141.6, 150.6, 172.5.

1,5-Bis(3-pyridinium chloride)-1,4-pentadien-3-one (31). 1,3-Acetone-dicarboxylic acid (27, 1.05 g, 7.2 mmol) was dissolved in ethanol (10 ml) and stirred for 15 min at room temperature. 3-Pyridinecarboxaldehyde (30, 1.36 ml, 14.4 mmol) was added dropwise and the mixture stirred for 2 hr at room temperature. Hydrochloric acid (5 ml) was added and the mixture stirred for 1 hr at 80° C. The resulting mixture was filtered to afford a solid. The crude solid was recrystallized from water/acetone to give 1.57 g (71%) of a yellow solid: mp >250° C. [expected mp >250° C.]; ¹H NMR: (D₂O) δ 7.40 (d, 2H, J=16.3 Hz), 7.78 (d, 2H, J=16.1 Hz), 8.02 (t, 2H, J=7.9 Hz), 8.70 (d, 2H, J=5.6 Hz), 8.79 (d, 2H, J=7.9 Hz), 8.98 (s, 2H); ¹³C NMR: (D₂O) δ 130.1, 132.8, 136.9, 139.9, 143.7, 144.3, 147.3, 193.0.

1,5-Bis(3-pyridyl)-1,4-pentadien-3-one (32). 1,5-Bis(3-pyridinium chloride)-1,4-pentadien-3-one (31, 0.50 g, 1.6 mmol) and sodium hydroxide (1.6 g, 40 mmol) were combined in water (40 ml) and stirred for 15 min at room temperature. The resulting mixture was extracted with ethyl acetate, washed with saturated sodium chloride, dried over magnesium sulfate, filtered and evaporated to afford a solid. The crude solid was recrystallized from ethyl acetate/hexane to give 0.31 g (81%) of a yellow solid: mp 148-149° C. [expected mp 150° C.]; ¹H NMR: δ 7.11 (d, 2H, J=16.1 Hz), 7.32 (m, 2H), 7.71 (d, 2H, J=15.9 Hz), 7.90 (d, 2H, J=6.2 Hz), 8.61 (d, 2H, J=4.6 Hz), 8.81 (s, 2H); ¹³C NMR: δ 123.7, 126.7, 130.3, 134.4, 139.9, 149.9, 151.1, 198.6.

1,5-Bis(2-thienyl)-1,4-pentadien-3-one (34). 2-Thiophenecarboxaldehyde (33, 0.50 ml, 5.3 mmol) and acetone (19, 0.20 ml, 2.7 mmol) were combined in ethanol (10 ml) and stirred for 10 min at room temperature. A solution of sodium hydroxide (0.30 g, 7.5 mmol) and water (10 ml) was added and the mixture stirred for 18 hr at room temperature. The resulting precipitate was filtered and recrystallized from ethanol/water to give 0.55 g (82%) of a yellow solid: mp 115-117° C. [expected mp 115-117° C.]; ¹H NMR: δ 6.80 (d, 2H, J=15.5 Hz), 7.06 (dt, 2H, J=3.6, 1.4 Hz), 7.31 (d, 2H, J=3.4 Hz), 7.39 (d, 2H, J=5.0 Hz), 7.82 (d, 2H, J=15.5 Hz); ¹³C NMR: δ 124.4, 128.2, 128.7, 131.7, 135.5, 140.2, 187.5.

1-(4-Hydroxy-3-methoxyphenyl)-1-buten-3-one (35a). 1-(4-Methoxymethyloxy-3-methoxyphenyl)-1-buten-3-one (35j, 0.40 g, 1.7 mmol) was dissolved in methanol (40 ml) and stirred for 15 min at 50° C. Hydrochloric acid (3 drops) was added and the mixture stirred for 18 hr at 65° C. The methanol was evaporated and the resulting residue extracted into ethyl acetate, washed with saturated sodium chloride, dried over magnesium sulfate, filtered and evaporated to afford a solid. The crude solid was chromatographed on silica gel with ethyl acetate/hexane to give 0.24 g (74%) of an orange-yellow solid: mp 120-122° C. [expected mp 128-129° C.]; ¹H NMR 8 2.34 (s, 3H), 3.91 (s, 3H), 5.98 (s, 1H), 6.56 (d, 1H, J=16.1 Hz), 6.91 (d, 1H, J=8.2 Hz), 7.04 (m, 2H), 7.43 (d, 1H, J=16.3 Hz); ¹³C NMR: δ 27.3, 56.0, 109.3, 114.8, 123.4, 124.9, 126.9, 143.6, 146.8, 148.2, 198.2.

1-(4-Methoxyphenyl)-1-buten-3-one (35e). 4-Methoxybenzaldehyde (1e, 0.63 ml, 5.2 mmol) and acetone (19, 4.00 ml, 54.0 mmol) were combined in ethanol (4 ml) and stirred for 15 min at room temperature. A solution of sodium hydroxide (0.40 g, 10.0 mmol) and water (4 ml) was added dropwise and the mixture stirred for 1 hr at room temperature. The resulting mixture was extracted into ethyl acetate, washed with saturated sodium chloride, dried over magnesium sulfate, filtered and evaporated to afford a solid. The crude solid was recrystallized from ether/hexane to give 0.57 g (62%) of a yellow solid: mp 71-73° C. [expected mp 68° C.]; ¹H NMR: δ 2.34 (s, 3H), 3.83 (s, 3H), 6.59 (d, 1H, J=16.3 Hz), 6.90 (d, 2H, J=8.7 Hz), 7.46 (d, 1H, J=16.3 Hz), 7.48 (d, 2H, J=8.7 Hz); ¹³C NMR: δ 27.5, 55.4, 114.4, 125.0, 127.1, 129.9, 143.1, 161.5, 198.1.

1-(4-Methoxymethyloxy-3-methoxyphenyl)-1-buten-3-one (35j). 4-Methoxy-methyloxy-3-methoxybenzaldehyde (1j, 2.30 g, 11.7 mmol) and acetone (19, 8.75 ml, 118.4 mmol) were combined in ethanol (20 ml) and stirred for 15 min at room temperature. A solution of sodium hydroxide (0.80 g, 20.0 mmol) and water (20 ml) was added and the mixture stirred for 1 hr at room temperature. The resulting mixture was extracted into ethyl acetate, washed with saturated sodium chloride, dried over magnesium sulfate, filtered and evaporated to afford a solid. The crude solid was recrystallized from hexane to give 2.70 g (97%) of a white solid: mp 73-75° C.; ¹H NMR: δ 2.34 (s, 3H), 3.48 (s, 3H), 3.89 (s, 3H), 5.24 (s, 2H), 6.58 (d, 1H, J=16.1 Hz), 7.07 (m, 2H), 7.13 (d, 1H, J=8.7 Hz), 7.43 (d, 1H, J=16.1 Hz); ¹³C NMR: δ 27.4, 55.9, 56.3, 95.2, 110.4, 115.9, 122.5, 125.7, 128.7, 143.1, 148.7, 149.8, 198.0.

1-(2-Hydroxyphenyl)-1-buten-3-one (35q). 2-Hydroxybenzaldehyde (1q, 0.90 ml, 8.4 mmol) and acetone (19, 1.24 ml, 16.8 mmol) were combined in ethanol (7 ml) and stirred for 15 min at room temperature. A solution of sodium hydroxide (0.5 g, 12.5 mmol) and water (2 ml) was added dropwise and the mixture stirred for 48 hr at room temperature. The mixture was neutralized with hydrochloric acid (1 N), extracted with ethyl acetate, washed with saturated sodium chloride, dried over magnesium sulfate, filtered and evaporated to afford a solid. The crude solid was recrystallized from tetrahydrofuran/hexane to give 0.36 g (26%) of a yellow solid: mp 136-137° C. [expected mp 139-140° C.]; ¹H NMR: δ 2.42 (s, 3H), 6.92 (m, 2H), 7.03 (d, 1H, J=16.5 Hz), 7.24 (dt, 1H, J=7.0, 1.4 Hz), 7.45 (d, 1H, J=7.7 Hz), 7.88 (d, 1H, J=16.3 Hz), 8.00 (s, 1H); ¹³C NMR: δ 26.8, 116.6, 120.5, 127.5, 129.5, 131.9, 141.0, 156.1, 156.1, 201.3.

1 -(4-Hydroxy-3-methoxyphenyl)-5-phenyl-1,4-pentadien-3-one (36a). 1-(4-Methoxymethyloxy-3-methoxyphenyl)-1-buten-3-one (35j, 1.00 g, 4.2 mmol) and benzaldehyde (1b, 0.46 ml, 4.5 mmol) were combined in ethanol (10 ml) and stirred for 15 min at room temperature. A solution of sodium hydroxide (0.30 g, 7.5 mmol) and water (10 ml) was added and the mixture stirred for 18 hr at room temperature. The resulting mixture was extracted into ethyl acetate, washed with saturated sodium chloride, dried over magnesium sulfate, filtered and evaporated to give 1.35 g (99%) of an oil which was used without purification. The oil (36j, 1.30 g, 4.0 mmol) was stirred in methanol (50 ml) for 15 min at 60° C. Concentrated hydrochloric acid (3 drops) was added and the solution stirred for 18 hr at 60° C. The methanol was evaporated and the resulting residue extracted into ethyl acetate, washed with saturated sodium chloride, dried over magnesium sulfate, filtered and evaporated to afford a semi-solid. The crude semi-solid was chromatographed on silica gel with ethyl acetate/hexane to give 0.41 g (36%) of a yellow oil; ¹H NMR δ 3.92 (s, 3H), 6.08 (s, 1H), 6.91 (d, 1H, J=16.1 Hz), 6.93 (d, 1H, J=8.2 Hz), 7.07 (d, 1H, J=15.9 Hz), 7.10 (s, 1H), 7.15 (d, 1H, J=8.0 Hz), 7.38 (m, 3H), 7.59 (m, 2H), 7.67 (d, 1H, J=15.9 Hz), 7.71 (d, 1H, J=15.9 Hz); ¹³C NMR: δ 56.0, 109.8, 114.9, 123.4, 125.3, 127.3, 128.3, 128.8, 130.3, 134.9, 142.8, 143.5, 146.8, 148.3, 188.7.

1-(4-Methoxyphenyl)-5-phenyl-1,4-pentadien-3-one (36e). 1-(4-Methoxy-phenyl)-1-buten-3-one (35e, 0.29 g, 1.6 mmol) was dissolved in methanol (5 ml) and stirred for 5 min at room temperature. A solution of sodium hydroxide (0.14 g, 3.5 mmol) and water (5 ml) was added and the mixture stirred for 30 min at room temperature. Benzaldehyde (1b, 0.17 ml, 1.7 mmol) was added dropwise and the mixture stirred for 18 hr at room temperature. The resulting precipitate was filtered and recrystallized from ethanol to give 0.41 g (94%) of a yellow solid: mp 85-89° C. [expected mp 118-119° C.]; ¹H NMR: δ 3.82 (s, 3H), 6.91 (d, 2H, J=8.5 Hz), 6.94 (d, 1H, J=15.9 Hz), 7.06 (d, 1H, J=16.1 Hz), 7.38 (m, 4H), 7.57 (m, 3H), 7.71 (dd, 2H, J=15.9, 2.0 Hz); ¹³C NMR: δ 55.4, 114.4, 123.3, 125.5, 127.4, 128.2, 128.8, 130.0, 130.2, 134.8, 142.6, 143.0, 161.5, 188.6.

2,6-Bis(4-hydroxy-3-methoxybenzylidene)cyclohexanone (38a). 2,6-Bis(4-methoxymethyloxy-3-methoxybenzylidene)cyclohexanone (38j, 0.49 g, 1.1 mmol) was dissolved in methanol (100 ml) and stirred for 15 min at room temperature. Concentrated hydrochloric acid (3 drops) was added and the mixture stirred for 3 hr at 60° C. The methanol was evaporated and the resulting residue extracted into ethyl acetate, washed with saturated sodium chloride, dried over magnesium sulfate, filtered and evaporated to afford a solid. The crude solid was recrystallized from ethanol to give 0.26 g (66%) of a yellow solid: mp 177-178° C. [expected mp 179-181° C.]; ¹H NMR: δ 1.79 (m, 2H), 2.90 (t, 4H, J=5.4 Hz), 3.89 (s, 6H), 5.88 (s, 2H), 6.91 (s, 2H), 6.96 (d, 2H, J=4.8 Hz), 7.06 (d, 2H, J=8.0 Hz), 7.72 (s, 2H); ¹³C NMR: δ 23.1, 28.5, 56.0, 113.2, 114.4, 124.4, 128.5, 134.2, 136.9, 146.2, 146.4, 172.8.

2,6-Bis(benzylidene)cyclohexanone (38b). Benzaldehyde (1b, 1.00 ml, 9.8 mmol) and cyclohexanone (37, 0.51 ml, 4.9 mmol) were combined in ethanol (10 ml) and stirred for 15 min at room temperature. A solution of sodium hydroxide (0.40 g, 10 mmol) and water (10 ml) was added and the mixture stirred for 18 hr at room temperature. The resulting precipitate was filtered and recrystallized from ethyl acetate to give 0.99 g (73%) of yellow crystals: mp 118-119° C. [expected mp 117° C.]; ¹H NMR: δ 1.77 (m, 2H), 2.92 (t, 4H, J=5.2 Hz), 7.39 (m, 10H), 7.80 (s, 2H); ¹³C NMR: δ 23.1, 28.5, 128.3, 128.5, 130.2, 135.9, 136.1, 136.8, 190.1.

2,6-Bis(4-methoxymethyloxy-3-methoxybenzylidene)cyclohexanone (38j). 4-Methoxymethyloxy-3-methoxybenzaldehyde (1j, 2.08, 10.1 mmol) and cyclohexanone (37, 0.55 ml, 5.3 mmol) were combined in ethanol (10 ml) and stirred for 15 min at room temperature. A solution of sodium hydroxide (0.40 g, 10.0 mmol) and water (10 ml) was added and the mixture stirred for 18 hr at room temperature. The resulting precipitate was filtered and recrystallized from ethyl acetate to give 1.66 g (69%) of a yellow solid: mp 73-75° C.; ¹H NMR: δ 1.81 (m, 2H), 2.90 (t, 4H, J=5.2 Hz), 3.52 (s, 6H), 3.91 (s, 6H), 5.26 (s, 4H), 7.05 (m, 4H), 7.17 (d, 2H, J=7.9 Hz), 7.74 (s, 2H); ¹³C NMR: δ 22.9, 28.4, 55.8, 56.1, 95.1, 114.1, 115.6, 123.4, 130.2, 134.7, 136.4, 146.8, 149.1, 190.3.

1,5-Diphenylpentan-3-one (39b). 1,5-Diphenyl-1,4-pentadien-3-one (20b, 1.00 g, 4.3 mmol) and palladium on activated carbon (0.25 g, 5%) were combined in ethyl acetate (50 ml). The mixture was placed under a hydrogen atmosphere (60 psi) on a Parr apparatus for 2 hr at room temperature. The resulting mixture was filtered through celite and the solvent evaporated to afford an oil. The crude oil was chromatographed on silica gel with ethyl acetate/hexane to give 0.82 g (80%) of a clear oil; ¹H NMR: δ 2.76 (t, 4H, J=7.6 Hz), 2.97 (t, 4H, J=7.4 Hz), 7.30 (m, 10H); ¹³C NMR: δ 29.6, 44.2, 125.8, 128.0, 128.2, 140.7, 208.4.

1,5-Diphenylpentan-3-ol (40b). 1,5-Diphenyl-1,4-pentadien-3-one (20b, 1.00 g, 4.3 mmol) and palladium on activated carbon (0.25 g, 5%) were combined in ethyl acetate (50 ml). The mixture was placed under a hydrogen atmosphere (60 psi) on a Parr apparatus for 2 hr at room temperature. The resulting mixture was filtered through celite and the solvent evaporated to afford an oil. The crude oil was chromatographed on silica gel with ethyl acetate/hexane to give 0.12 g (12%) of a white solid: mp 47-49° C. [expected mp 45-46° C.]; ¹H NMR: δ 1.86 (m, 4H), 2.77 (m, 4H), 3.70 (m, 1H), 7.31 (m, 10H); ¹³C NMR: δ 32.1, 39.2, 70.8, 125.6, 128.3, 142.0.

trans,trans-1,2,4,5-Diepoxy-1,5-diphenylpentan-3-one (42b) and cis,cis-1,2,4,5-diepoxy-1,5-diphenylpentan-3-one (43b). Potassium fluoride dihydrate (9.40 g, 0.1 mol) and neutral aluminum oxide (10.0 g, 98.1 mmol) were combined in water (100 ml) and stirred for 30 min at room temperature. The water was evaporated and the resulting material placed in an oven for 5 days at 125° C. A suspension of potassium fluoride-alumninum oxide (0.48 g, 3.0 mmol) in acetonitrile (6 ml) was added to a solution of 1,5-diphenyl-1,4-pentadien-3-one (20b, 0.47 g, 2.0 mmol) in acetonitrile (1.0 ml) and the mixture stirred for 15 min at room temperature. t-Butyl hydroperoxide (41, 1.7 ml, 17.7 mmol, 70% solution in water) was extracted with dichloroethane (6 ml), dried over magnesium sulfate, filtered, added to the suspension and stirred for 30 min at room temperature. The resulting mixture was filtered and the solvent evaporated to afford a solid. The crude solid was chromatographed on silica gel with ethyl acetate/hexane to give a mixture of isomers 42b and 43b. The crude solid was recrystallized twice from ethanol to give 0.21 g (39%) of 42b as white crystals: mp 117-119° C. [expected mp 118-118.5° C.]; ¹H NMR: δ 3.80 (d, 2H, J=1.4 Hz), 4.09 (d, 2H, J=1.4 Hz), 7.30 (m, 10H); ¹³C NMR: δ 59.0, 60.9, 125.7, 128.7, 129.2, 134.5, 199.0. The filtrate was evaporated to give 0.25 g (47%) of 43b as a yellow oil; ¹H NMR: δ 3.72 (d, 2H, J=1.6 Hz), 4.18 (d, 2H, J=1.6 Hz), 7.33 (m, 10H); ¹³C NMR: δ 58.9, 60.3, 125.8, 128.7, 129.2, 134.5, 199.0.

4-Methoxymethyloxy-3-methoxyacetophenone (44j). 4-Hydroxy-3-methoxy-acetophenone (44a, 2.5 g, 15 mmol) and potassium carbonate (15.0 g, 108.5 mmol) were combined in dimethyl formamide (50 ml) and stirred for 15 min at room temperature. Chloromethyl methyl ether (18, 1.25 ml, 16.5 mmol) was added and stirring was continued for 4 hr at room temperature. Potassium carbonate was filtered and the filtrate extracted into ethyl acetate, washed with saturated sodium chloride, dried over magnesium sulfate, filtered and evaporated to 3.09 g (98%) of an oil; ¹H NMR: δ 2.38 (s, 3H), 3.33 (s, 3H), 3.75 (s, 3H), 5.12 (s, 2H), 6.99 (d, 1H, J=8.9 Hz), 7.35 (dd, 1H, J=6.6, 2.0 Hz), 7.82 (s, 1H).

1,3-Bis(4-hydroxy-3-methoxyphenyl)-2-propen-1-one (45a). 4-Methoxy-methyloxy-3-methoxyacetophenone (44j, 2.14 g, 10.2 mmol) and barium hydroxide octahydrate (3.25 g, 10.3 mmol) were combined in methanol (50 ml) and stirred for 15 min at 50° C. 4-Methoxymethyloxy-3-methoxybenzaldehyde (1j, 2.00 g, 10.2 mmol) was added and the mixture stirred for 18 hr at 50° C. The methanol was evaporated and the resulting residue extracted into ethyl acetate, washed with saturated sodium chloride, dried over magnesium sulfate, filtered and evaporated to give 3.90 g (99%) of an oil which was used without purification: ¹H NMR: 8 3.50 (s, 6H), 3.92 (s, 3H), 3.94 (s, 3H), 5.25 (s, 2H), 5.30 (s, 2H), 7.18 (m, 4H), 7.39 (d, 1H, J=15.5 Hz), 6.61 (m, 2H), 7.73 (d, 1H, J=15.5 Hz). The oil (45j, 1.10 g, 2.8 mmol) was stirred in methanol (50 ml) for 5 min at 60° C. Concentrated hydrochloric acid (3 drops) was added and the mixture stirred for 3 hr at 60° C. The methanol was evaporated and the resulting residue was extracted into ethyl acetate, washed with saturated sodium chloride, dried over magnesium sulfate, filtered and evaporated to afford an oil. The crude oil was chromatographed on silica gel to give 0.47 (55%) of a yellow solid: mp 111-114° C. [expected mp 126-128° C.]; ¹H δ 3.94 (s, 3H), 3.95 (s, 3H), 6.00 (s, 1H), 6.19 (s, 1H), 6.95 (m, 2H), 7.11 (d, 1H, J=1.6 Hz), 7.20 (dd, 1H, J=8.3, 1.6 Hz), 7.38 (d, 1H, J=15.5 Hz), 7.61 (m, 2H), 7.73 (d, 1H, J=15.7 Hz); ¹³C NMR: δ 56.0, 56.1, 110.0, 110.5, 113.6, 114.8, 119.2, 123.0, 123.4, 127.6, 131.1, 144.2, 146.7, 146.8, 148.0, 150.1, 188.4.

1,3-Diphenyl-propenone (45b). Acetophenone (44b, 1.20 ml, 10.3 mmol) and sodium hydroxide (0.40 g, 10.0 mmol) were combined in methanol (10 ml) and stirred for 30 min at room temperature. A solution of benzaldehyde (1b, 1.02 ml, 10.0 mmol) and methanol (10 ml) was added dropwise and the mixture stirred for 21 hr at room temperature. Water (25 ml) was added and the mixture neutralized with hydrochloric acid (1 N). The mixture was extracted into ethyl acetate, washed with saturated sodium chloride, dried over magnesium sulfate, filtered and evaporated to afford a semi-solid. The crude semi-solid was chromatographed on silica gel with ethyl acetate/hexane to give a solid. The solid was recrystallized from hexane to give 1.11 g (53%) of a pale yellow solid: mp 52-54° C. [expected mp 55-58° C.]; ¹H NMR: 8 7.40 (m, 3H), 7.46 (t, 1H, J=1.6 Hz), 7.63 (m, 5H), 7.81 (d, 1H, J=15.7 Hz), 8.02 (dd, 2H, J=8.0, 1.2 Hz); ¹³C NMR: δ 122.1, 128.3, 128.4, 128.5, 128.8, 130.4, 132.6, 134.8, 138.2, 144.7, 190.3. 1-(4-Hydroxy-3-methoxyphenyl)-3-phenyl-2-propen-1-one (46a). 4-Methoxy-methyloxy-3-methoxyacetophenone (44j, 2.66 g, 12.7 mmol) and barium hydroxide octahydrate (4.00 g, 12.7 mmol) were combined in methanol (50 ml) and stirred for 5 min at 50° C. Benzaldehyde (1b, 1.30 ml, 12.8 mmol) was added and the mixture stirred for 8 hr at 50° C. The methanol was evaporated and the resulting residue was extracted into ethyl acetate, washed with saturated sodium chloride, dried over magnesium sulfate, filtered and evaporated to give 3.38 g (90%) of an oil which was used without purification; ¹H NMR: δ 3.48 (s, 3H), 3.92 (s, 3H), 5.28 (s, 2H), 7.18 (d, 2H, J=8.9 Hz), 7.36 (m, 3H), 7.51 (d, 1H, J=15.7 Hz), 7.60 (m, 3H), 7.77 (d, 1H, J=15.7 Hz). The oil (46j, 3.35 g, 11.2 mmol) was stirred in methanol (75 ml) for 10 min at 50° C. Concentrated hydrochloric acid (3 drops) was added and the mixture stirred for 3 hr at 50° C. The methanol was evaporated and the resulting residue was extracted into ethyl acetate, washed with saturated sodium chloride, dried over magnesium sulfate, filtered and evaporated to afford an oil. The crude oil was distilled bulb to bulb to give 2.12 (74%) of a yellow solid: mp 61-64° C. [expected mp 63-66° C.]; ¹H NMR δ 3.95 (s, 3H), 6.29 (s, 1H), 6.98 (d, 1H, J=8.3 Hz), 7.38 (m, 2H), 7.53 (d, 1H, J=15.5 Hz), 7.62 (m, 5H), 7.79 (d, 1H, J=15.5 Hz); ¹³C NMR: 6 56.1, 110.5, 113.8, 121.6, 123.6, 128.3, 128.8, 130.2, 130.9, 135.0, 143.8, 146.8, 150.4, 188.4.

1-(4-Carboxyphenyl)-3-phenyl-2-propen-1-one (46ad). 4-Acetylbenzonitrile (44al, 1.00 g, 6.9 mmol) and sulfuric acid (4 ml) were combined in water (4 ml) and the mixture stirred for 2.5 hr at reflux. The resulting mixture was extracted into ethyl acetate, washed with saturated sodium chloride, dried over magnesium sulfate, filtered and evaporated to give 0.98 g (87%) of compound 44ad as a white solid: mp 204° C.; ¹H NMR: (DMSO) δ 2.61 (s, 3H), 8.04 (s, 4H), 13.23 (s, 1H). The solid (44ad, 0.50 g, 3.0 mmol) and sodium hydroxide (0.29 g, 7.3 mmol) were combined in water (4 ml) and ethanol (4 ml) and stirred for 30 min at room temperature. Benzaldehyde (1b, 0.31 ml, 3.1 mmol) was added and the mixture stirred for 48 hr at room temperature. The resulting mixture was acidified with hydrochloric acid (1 I N), extracted into ethyl acetate, washed with saturated sodium chloride, dried over magnesium sulfate, filtered and evaporated to a afford a solid. The crude solid was recrystallized from ethyl acetate to give 0.54 g (70%) of a yellow solid: mp 217-220° C. [expected mp 217-220° C.]; ¹H NMR: (DMSO) δ 7.45 (m, 5H), 7.76 (d, 1H, J=16.1 Hz), 7.93 (d, 1H, J=15.5 Hz), 8.09 (d, 2H, J=7.9 Hz), 8.23 (d, 2H, J=7.6 Hz) 13.34 (s, 1H); ¹³C NMR: (DMSO) δ 121.9, 128.5, 128.8, 128.9, 129.4, 130.6, 134.3, 134.4, 140.6, 144.6, 166.4, 188.8.

1-(2,4-Dimethylphenyl)-3-phenyl-2-propen-1-one (46ak). 2,4-Dimethyl-acetophenone (44ak, 1.48 g, 10.0 mmol) and sodium hydroxide (0.54 g, 13.5 mmol) were combined in methanol (30 ml) and stirred for 30 min at room temperature. A solution of benzaldehyde (1b, 1.02 ml, 10.0 mmol) and methanol (30 ml) was added dropwise and the mixture stirred for 18 hr at room temperature. Water (25 ml) was added and the mixture neutralized with hydrochloric acid (1N). The mixture was extracted into ethyl acetate, washed with saturated sodium chloride, dried over magnesium sulfate, filtered and evaporated to afford an oil. The crude oil was distilled bulb to bulb to give 1.98 g (84%) of a yellow oil: [expected mp 68° C.]; ¹H NMR δ 2.37 (s, 3H), 2.44 (s, 3H), 7.05 (m, 2H), 7.16 (d, 1H, J=16.1 Hz), 7.38 (m, 3H), 7.49 (d, 1H, J=15.9 Hz), 7.54 (m, 3H); ¹³C NMR: δ 20.4, 21.4, 126.0, 126.6, 128.2, 128.5, 128.8, 130.4, 132.2, 134.7, 136.1, 137.4, 140.8, 145.0, 195.6.

1-(4-Cyanophenyl)-3-phenyl-2-propen-1-one (46al). 4-Acetylbenzonitrile (44al, 1.00 g, 6.9 mmol), sodium hydroxide (0.40 g, 10.0 mmol) and water (20 ml) were combined in ethanol (20 ml) and stirred for 15 min at room temperature. Benzaldehyde (1b, 0.70 ml, 6.9 mmol) was added and the mixture stirred for 2 hr at room temperature. The resulting mixture was filtered and recrystallized from ethanol to give 1.46 g (91%) of a yellow solid: mp 120° C. [expected mp 119-120° C.]; ¹H NMR: δ 7.34 (m, 3H), 7.62 (m, 2H), 7.80 (m, 4H), 8.06 (d, 2H, J=8.1 Hz); ¹³C NMR: δ 115.9, 117.9, 121.1, 128.6, 128.8, 129.0, 131.0, 132.4, 134.3, 141.4, 146.4, 188.9.

3-(4-Hydroxy-3-methoxyphenyl)-1-phenyl-2-propen-1-one (48a). 4-Hydroxy-3-methoxybenzaldehyde (1a, 2.02 g, 13.3 mmol) and pyridinium p-toluenesulfonate (90 mg, 0.4 mmol) were combined in dichloromethane (60 ml) and stirred for 5 min at room temperature. A solution of 3,4-dihydropyran (47, 3.6 ml, 39.5 mmol) in dichloromethane (20 ml) was added dropwise and the mixture stirred for 5 hr at room temperature. The resulting mixture was washed with saturated sodium chloride, dried over magnesium sulfate, filtered and evaporated to afford an oil. The crude oil was chromatographed on silica gel with ethyl acetate/hexane to give 2.61 g (85%) of a clear oil. The oil (1am, 1.01 g, 4.3 mmol) and barium hydroxide octahydrate (1.03 g, 3.3 mmol) were combined in methanol (26 ml) and stirred for 15 min at room temperature. Acetophenone (44b, 0.30 ml, 2.6 mmol) was added and the mixture stirred for 16 hr at 50° C. The methanol was evaporated, water was added and the mixture acidified with hydrochloric acid (6 N). The resulting mixture was extracted into ethyl acetate, washed with saturated sodium chloride, dried over magnesium sulfate, filtered and evaporated to afford an oil. The crude oil was triturated with hexane to give a solid. The crude solid was recrystallized from ethyl acetate/hexane to give 0.39 g (45%) of a yellow solid: mp 87-88° C. The solid (48am, 0.39 g, 1.2 mmol) and p-toluenesulfonic acid (0.10 g, 0.6 mmol) were combined in methanol (50 ml) and stirred for 4 hr at room temperature. The methanol was evaporated and water was added. The mixture was neutralized with saturated sodium bicarbonate and extracted into ethyl acetate. The ethyl acetate was washed with water, dried over magnesium sulfate, filtered and evaporated to afford an oil. The crude oil was chromatographed on silica gel with ethyl acetateihexane to afford a solid. The crude solid was recrystallized from hexane to give 0.18 g (62%) of a yellow solid: mp 81-84° C. [expected mp 85-90° C.]; ¹H NMR: δ 3.92 (s, 3H), 5.96 (s, 1H), 6.94 (d, 2H, J=8.1 Hz), 7.11 (s, 1H), 7.21 (d, 1H, J=7.6 Hz), 7.35 (d, 1H, J=15.9 Hz), 7.51 (m, 2H), 7.73 (d, 1H, J=15.5 Hz), 7.99 (d, 1H, J=7.0 Hz); ¹³C NMR: δ 56.1, 110.0, 114.8, 119.8, 123.3, 127.4, 128.4, 128.5, 132.5, 138.5, 145.1, 146.7, 148.2, 190.5.

3-(4-Carboxyphenyl)-1-phenyl-2-propen-1-one (48ad). Acetophenone (44b, 0.50 ml, 4.3 mmol) and sodium hydroxide (0.50 g, 12.5 mmol) were combined in ethanol (2 ml) and water (2 ml) and stirred for 30 min at room temperature. 4-Formylbenzoic acid (1ad, 0.71 g, 4.7 mmol) was added and the mixture stirred for 48 hr at room temperature. Water (25 ml) was added, the mixture acidified with hydrochloric acid (1 N) and the resulting precipitate was filtered and recrystallized from ethyl acetate to give 0.65 g (60%) of a white solid: mp 222-224° C. [expected mp 227-229° C.]; ¹H NMR: (DMSO) δ 7.67 (m, 4H), 7.99 (m, 4H), 8.18 (m, 3H), 13.14 (s, 1H); ¹³C NMR: (DMSO) δ 124.2, 128.5, 128.7, 128.8, 129.6, 132.1, 133.2, 137.3, 138.7, 142.4, 166.7, 189.0.

1,3-Diphenylpropane-1,3-dione (50b). Methanol (0.26 ml, 6.4 mmol) and sodium (0.14 g, 6.1 mmol) were combined in xylene (60 ml) and stirred for 20 min at room temperature. Methyl benzoate (49, 2.47 ml, 19.7 mmol) and acetophenone (0.58 ml, 5.0 mmol) were added and the mixture stirred for 6 hr at 140° C. The mixture was cooled to room temperature and hydrochloric acid (10 ml, 6 N) was added and stirred for 15 min. The resulting mixture was extracted into ethyl acetate, washed twice with water, twice with saturated sodium bicarbonate and twice with water. The ethyl acetate was dried over magnesium sulfate, filtered and evaporated to afford an oil. The crude oil was chromatographed on silica gel with ethyl acetate/hexane to give a solid. The solid was recrystallized from methanol to give 0.71 g (63%) of a pink-orange solid: mp 70-71° C. [expected mp 77-78° C.]; ¹H NMR: δ 6.85 (s, 1H), 7.51 (m, 6H), 7.98 (d, 4H, J=6.8 Hz); ¹³C NMR: δ 93.1, 127.1, 128.6, 132.4, 135.5, 185.6.

2,6-Diphenyl-1-methyl-4-piperidone (52b). 1,5-Diphenyl-1,4-pentadien-3-one (20b, 4.00 g, 17.1 mmol) was dissolved in dimethyl formamide (60 ml). Methylamine (51, 6.0 ml, 70.0 mmol, 40% in water) was added and the mixture stirred for 96 hr at room temperature. The mixture was poured into water (250 ml) and stirred for 1 hr at room temperature. The resulting mixture was extracted into ethyl ether, washed with saturated sodium chloride, dried over magnesium sulfate, filtered and evaporated to afford a solid. The crude solid was recrystallized from ethanol to give 2.74 g (60%) of a white solid: mp 147-149° C. [expected mp 148-150° C.]; ¹H NMR δ 1.82 (s, 3H), 2.50, (dd, 2H, J=12.3, 2.5 Hz), 2.82 (t, 2H, J=13.3 Hz), 3.45 (dd, 2H, J=12.9, 2.4 Hz), 7.34, (m, 10); ¹³C NMR: δ 40.8, 50.8, 70.2, 127.0, 127.6, 128.8, 143.1, 206.8.

2,6-Bis(2-methoxyphenyl)-1-methyl-4-piperidone (52c). 1,5-Bis(2-methoxy-phenyl)-1,4-pentadien-3-one (20c, 0.26 g, 0.9 mmol) was dissolved in dimethyl formamide (5 ml). Methylamine (51, 0.40 ml, 4.6 mmol, 40% in water) was added and the mixture stirred for 24 hr at room temperature. The mixture was poured into water (50 ml) and stirred for 24 hr at room temperature. The resulting mixture was extracted into ethyl acetate, washed with saturated sodium chloride, dried over magnesium sulfate, filtered and evaporated to afford a solid. The solid was recrystallized twice from ethanol to give 0.16 g (55%) of a white solid: mp 146-148° C.; ¹H NMR δ 1.89 (s, 3H), 2.50 (d, 2H, J=13.7 Hz), 2.65 (t, 2H, J=11.9 Hz), 3.82 (s, 6H), 4.11 (d, 2H, J=11.5 Hz), 6.87 (d, 2H, J=8.3 Hz), 7.03 (t, 2H, J=7.2 Hz), 7.23 (t, 2H, J=5.8 Hz), 7.72 (d, 2H, J=7.6 Hz); ¹³C NMR: δ 40.3, 49.2, 55.4, 61.2, 110.7, 121.0, 127.6, 127.8, 131.5, 156.3, 208.1; Exact mass calcd for C₂₀H₂₃NO₃: 325.1678, observed (M+H) 326.1754.

2,6-Bis(4-methoxyphenyl)-1-methyl-4-piperidone (52e). 1,5-Bis(4-methoxy-phenyl)-1,4-pentadien-3-one (20e, 0.40 g, 1.4 mmol) was dissolved in dimethyl formamide (10 ml). Methylamine (51, 0.75 ml, 8.7 mmol, 40% in water) was added and the mixture stirred for 24 hr at room temperature. The mixture was poured into water (50 ml) and stirred for 2 hr at room temperature. The resulting mixture was extracted into ethyl acetate, washed with saturated sodium chloride, dried over magnesium sulfate, filtered and evaporated to afford a solid. The crude solid was chromatographed on silica gel with ethyl acetate/hexane to afford a solid that was recrystallized from ethanol to give 0.30 g (68%) of a white solid: mp 141-143° C. [expected mp 129-130° C.]; ¹H NMR δ 1.77, (s, 3H), 2.45 (d, 2H, J=14.5 Hz), 2.78 (t, 2H, J=12.9 Hz), 3.33 (d, 2H, J=11.9 Hz), 3.79 (s, 6H), 6.88 (d, 4H, J=8.5 Hz), 7.32 (d, 4H, J=8.5 Hz); ¹³C NMR: δ 40.6, 50.9, 55.3, 69.5, 114.1, 128.0, 135.3, 158.9, 207.1.

2,6-Bis(4-methylphenyl)-1-methyl-4-piperidone (52aa). 1,5-Bis(4-methyl-phenyl)-1,4-pentadien-3-one (20aa, 0.32 g, 1.2 mmol) was dissolved in dimethyl formamide (9 ml). Methylamine (51, 0.5 ml, 5.8 mmol, 40% in water) was added and the mixture stirred for 72 hr at room temperature. The mixture was poured into water (50 ml) and stirred for 2 hr at room temperature. The resulting mixture was extracted into ethyl acetate, washed with saturated sodium chloride, dried over magnesium sulfate, filtered and evaporated to afford a solid. The crude solid was chromatographed on silica gel with ethyl acetate/hexane to afford a solid that was recrystallized from ethanol to give 0.26 g (75%) of a white solid: mp 120-121° C. [expected mp 105-107° C.]; ¹H NMR δ 1.79 (s, 3H), 2.36 (s, 6H), 3.10 (d, 2H, J=14.9 Hz), 2.79 (t, 2H, J=13.1 Hz), 3.35 (dd, 2H, J=11.9, 2.4 Hz), 7.15 (d, 4H, J=8.0 Hz), 7.21 (d, 4H, J=7.9 Hz); ¹³C NMR: δ 21.2, 40.7, 50.9, 70.0, 126.9, 129.4 137.2, 140.2, 207.1.

2,6-Bis(2-methylphenyl)-1-methyl-4-piperidone (52ac). 1,5-Bis(2-methyl-phenyl)-1,4-pentadien-3-one (20ac, 0.50 g, 1.9 mmol) was dissolved in dimethyl formamide (10 ml). Methylamine (51, 1.0 ml, 11.6 mmol, 40% in water) was added and the mixture stirred for 24 hr at room temperature. The mixture was poured into water (50 ml) and stirred for 2 hr at room temperature. The resulting mixture was extracted into ethyl acetate, washed with saturated sodium chloride, dried over magnesium sulfate, filtered and evaporated to afford a solid. The crude solid was chromatographed on silica gel with ethyl acetate/hexane to afford a solid that was recrystallized from ethanol to give 0.29 g (52%) of a white solid: mp 155-157° C.; ¹H NMR δ 1.82, (s, 3H), 2.40, (s, 6H), 2.44 (d, 2H, J=11.9 Hz), 2.77 (t, 2H, J=13.3 Hz), 3.74 (d, 2H, J=11.9 Hz), 7.16 (m, 6H), 7.67, (d, 2H, J=7.0 Hz); ¹³C NMR: δ 19.5, 39.9, 49.3, 65.8, 126.7, 126.9, 130.6, 134.8, 140.9, 207.2; Exact mass calcd for C₂₀H₂₃NO: 293.1779, observed (M+H) 294.1856.

2,6-Bis(2-naphthyl)-1-methyl-4-piperidone (53). 1,5-Bis(2-naphthyl)-1,4-pentadien-3-one (25, 0.82 g, 2.5 mmol) was dissolved in dimethyl formamide (15 ml). Methylamine (51, 1.30 ml, 15.1 mmol, 40% in water) was added and the mixture stirred for 72 hr at room temperature. The mixture was poured into water (100 ml) and stirred for 24 hr at room temperature. The resulting mixture was extracted into ethyl acetate, washed with saturated sodium chloride, dried over magnesium sulfate, filtered and evaporated to afford a solid. The crude solid was chromatographed on silica gel with ethyl acetate/hexane to afford a solid that was recrystallized twice from ethanol to give 0.20 g (22%) of a white solid: mp 209-212° C.; 1H NMR δ 1.89 (s, 3H), 2.59 (d, 2H, J=13.9 Hz), 2.97 (t, 2H, J=11.3 Hz), 3.66 (d, 2H, J=11.7 Hz), 7.49 (m, 4H), 7.81 (m, 10H); ¹³C NMR: δ 41.1, 50.7, 70.3, 124.6, 125.9, 126.0, 126.2, 127.6, 127.7, 128.9, 133.0, 133.4, 140.4, 206.6; Exact mass calcd for C₂₆H₂₃NO: 365.1780, observed (M+H) 366.1852.

### Example 2

### Antioxidant Activity of Curcumin Derivatives

It has been suggested that the antioxidant activity of curcumin depends on the phenolic groups (Barclay et al., Organic Lett. 2000, 2(18), 2841-2843; Priyadarsini et al., Free Radical Biol. Med. 2003, 35(5), 475-484). However, other studies support the conclusion that the central methylene hydrogens of curcumin are important for antioxidant activity (Jovanovic et al., J. Am. Chem. Soc. 2001, 123(13), 3064-3068). More recently it has been demonstrated that both the central methylene hydrogens and the phenolic hydrogens may be involved in the mechanism of formation of the phenoxy radical, depending upon reaction conditions (Litwinienko et al., J. Org. Chem. 2004, 69(18), 5888-5896). The library consisting of three series of analogs examined the role of the enone functionality in aryl systems where the spacer is 7-carbons (as in curcumin), 5-carbons or 3-carbons in length. In addition, the importance of aryl ring substituents including phenolic groups was assessed as well as the importance of the central methylene hydrogens of curcumin. The antioxidant activities of the curcumin analogs were determined in two standard assays. There are multiple standardized methods to determine anti-oxidant activities, and it is recommended that at least two different procedures be used (Barclay et al., Organic Lett. 2000, 2(18), 2841-2843). The first assay was the Total Radical-trapping Anti-oxidant Parameter assay (TRAP assay) and the second assay was the Ferric Reducing/Anti-oxidant Power assay (FRAP assay).

TRAP Assay

The first procedure called for antioxidant activity to be measured as the ability of the analogs to react with the pre-formed radical monocation of 2,2′-azinobis-(3-ethylbenzothiazoline)-6-sulfonic acid (ABTS^{.+}). This assay is also known as the Total radical-trapping anti-oxidant parameter assay (TRAP assay). For the TRAP assay (Re et al., Free Rad. Biol. Med. 1999, 26, 1231-1237), 2,2′-azino-bis(3-ethylbenzthiazoline)-6-sulfonic acid (ABTS, 1.8 mM) was reacted with potassium persulfate (0.63 mM) in double distilled water, at room temperature in the dark, overnight, to generate the dark blue colored ABTS^{.+} radical cation, which has a maximum absorption at 734 nm. Just before the experiment, ABTS^{.+} was diluted with absolute ethanol to an absorbance of approximately 0.7 at 734 nm. ABTS^{.+} (1 ml) was added to curcumin or its analogs (10 μM in ethanol) and mixed by vortexing. The turquoise colored reaction was allowed to stabilize for 5 min and the absorbance monitored on a Perkin Elmer UV/Vis Lambda 2S. The activities of curcumin and its analogs were determined by their abilities to quench the color of the radical cation. The synthetic analog of α-tocopherol (vitamin E), Trolox, was used as a reference standard (10 μM in ethanol).

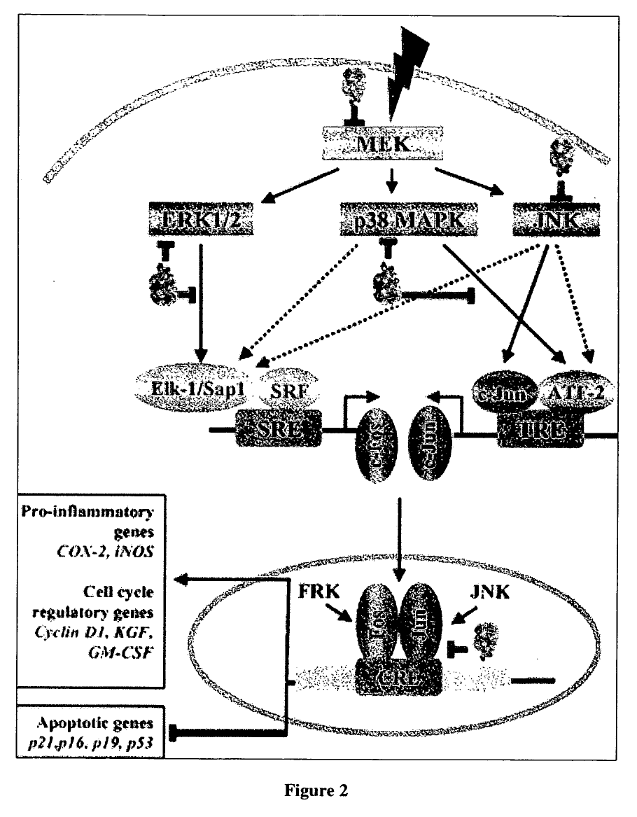

The first assay, the TRAP assay, determines the analogs abilities to reduce a radical cation generated from 2,2′-azino-bis(3-ethylbenzthiazoline)-6-sulfonic acid (ABTS). The following FIGS. (2A-2C) show the analogs active in the TRAP assay. The active analogs in FIGS. 2A-2C are arranged from highly active on the left to slightly active on the right.

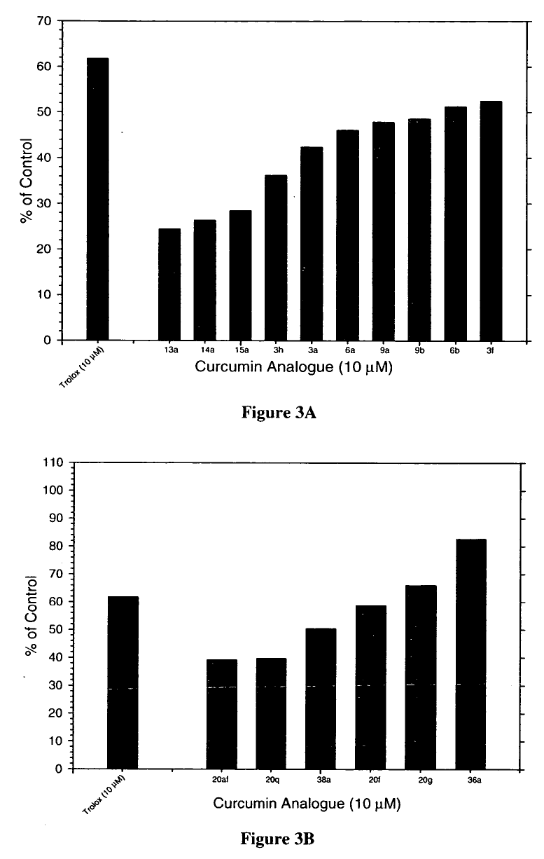

Active analogs retaining a 7-carbon spacer as in curcumin are shown in FIG. 3A. Four analogs, 13a, 14a, 15a and 3h, in this series were found to be more active than curcumin (3a). Of the ten active analogs in this series, eight retain phenolic groups as in curcumin. The three best analogs, 13a, 14a and 15a, not only contained phenolic groups but also contained a saturated spacer between the aryl rings. It is also evident that a central methylene substituent is favorable in analogs displaying antioxidant activity as six of the ten analogs in FIG. 3A contain a central methylene substituent.

Active analogs in series 2, which have a 5-carbon spacer, are shown in FIG. 3B. Two analogs, 20af and 20q, in this series were found to be more active than curcumin. Of the six active analogs in this series, five contain phenolic groups. The best analog, 20af, is a tetraphenol.

Active analogs in series 3, which have a 3-carbon spacer, are shown in FIG. 3C. Only one analog, 45a, in this series was found to be more active than curcumin. All five of the active analogs in this series contain phenolic groups.

FRAP Assay

Anti-oxidant activity was also measured in the Ferric reducing/anti-oxidant power assay (FRAP assay) in which the analogs are reacted with a ferric tripyridyltriazine complex. For the FRAP assay (Benzie et al., Meth. Enzymol. 1999, 299, 15-27), the ferric complex was prepared at room temperature by reaction of ferric chloride (16.7 mM) and 2,4,6-tripyridyl-s-triazine (8.33 mM) in acetate buffer (0.25 M) to pH 3.6. The FRAP reagent was used immediately after preparation. The ferric complex (1 ml) was added to curcumin or its analogs (10 μM in ethanol). The reaction was mixed by vortexing, allowed to stabilize for 5 min and the absorbance recorded on a Perkin Elmer UV/Vis Lambda 2S. The activities of curcumin and its analogs were determined by their abilities to reduce the ferric complex to a ferrous complex, monitored by the formation of the purple colored ferrous complex at 593 nm. The synthetic analog of α-tocopherol (vitamin E), Trolox, was used as a reference standard (10 μM in ethanol).

The second assay used, the FRAP assay, determines the analogs abilities to reduce a Fe(III) tripyridyltriazine complex to a Fe(II) tripyridyltriazine complex. The following FIGS. (3A-3C) show analogs active in the FRAP assay. The active analogs in FIGS. 3A-3C are arranged from highly active on the left to slightly active on the right.

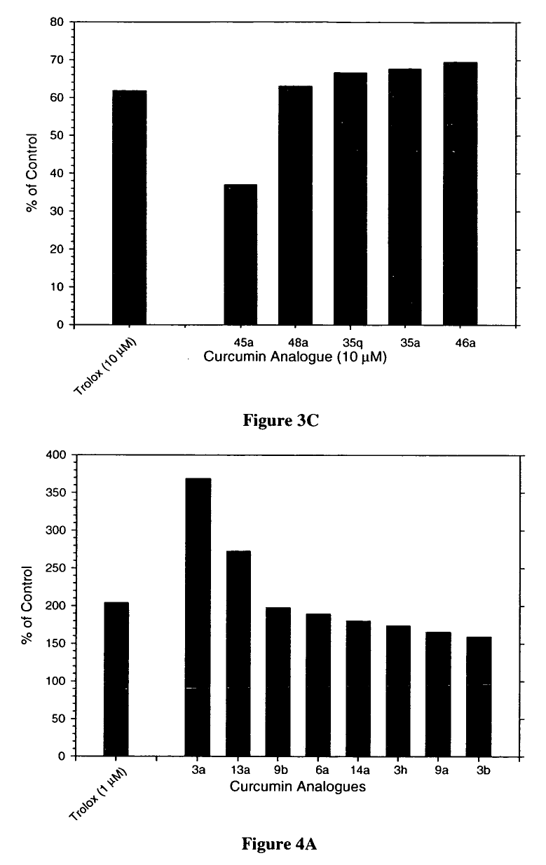

Active analogs retaining a 7-carbon spacer as in curcumin are shown in FIG. 4A. Curcumin (3a) displayed the most antioxidant activity in this series. Analog 13a, the reduced form of curcumin, also displayed potent antioxidant activity. Six of the eight active analogs in this series contain phenolic groups as in curcumin and four analogs contain substituents on the central methylene carbon.

Active analogs in series 2 are shown in FIG. 4B. Only one analog, 20af, in this series was found to be more active than curcumin. The two best analogs, 20af and 38a, in this series contain phenolic groups. However, contrary to any of the previous antioxidant results, only three of the twelve most active analogs in this series contain phenolic groups. There is currently no explanation as to why nine of the top twelve analogs in this series contain no phenolic groups and further investigation is necessary.

Active analogs in series 3 are shown in FIG. 4C. No analog in this series was more active than curcumin. Three of the five active analogs, 45a, 35a and 48a, in this series contain phenolic groups and a fourth, 46ad, contains an acidic carboxylic acid proton.

Most analogs that display antioxidant activity retain phenolic groups. Eighteen of the twenty one active analogs in the TRAP assay and twelve of the seventeen active analogs (minus the eight least active in the 5-carbon series) contain phenolic groups. This indicates that a phenolic substituent is desirable for antioxidant activity but not essential. Analogs in all three series were found to contain antioxidant activity with seven analogs in the 7-carbon series, three in the 5-carbon series and four in the 3-carbon series displaying activity in both the TRAP and FRAP assays.

### Example 3

### Inhibition of NF-κB Activity by Curcumin Derivatives

Curcumin and its analogs were screened for activity against NF-κB by a cellular assay using the NF-κB stable cell line (293T/NF-κB-luc). The cell line is derived from human 293T embryonic kidney cells expressing the large T antigen containing a chromosomal integration of a luciferase reporter construct regulated by 6 copies of the NF-κB response element (Panomics, Inc.). This stable clonal cell line is obtained by co-transfection of pNF-κB-luc and pTK-hyg containing plasmids followed by the addition of hygromycin (200 μg/ml) to maintain cell selection.

The cell line was grown in a humidified atmosphere at 37° C. in 5% CO₂/95% air and maintained in Dulbecco's Modified Eagle's Medium (DMEM-high glucose containing 4 mM glutamine) containing fetal bovine serum (FBS, 10%), sodium pyruvate (1 mM), penicillin (100 units/ml), streptomycin (100 μg/ml) and hygromycin (100 μg/ml) to maintain cell selection (Gibco/Invitrogen).

The 293T/NF-κB-luc cells were re-plated 24 hr prior to treatment, into 24-well cell culture plates in media without hygromycin, to prevent it from interfering with the assay. The cells were then allowed to grow and attach, to the wells, for 24 hr in a humidified atmosphere at 37° C. in 5% CO₂/95% air. After 24 hr, the cells had reached approximately 70% confluency and were given fresh media (1 ml) 1 hr prior to treatment with curcumin and its analogs. The cells were then re-given media (1 ml) with or without recombinant tumor necrosis factor alpha (TNFα, 20 ng/ml in phosphate buffered saline (PBS) at pH 7.4 containing 0.1% human serum albumin, R&D Biosciences/Clontech) followed by immediate treatment with curcumin or its analog (10 μM in DMSO). The cells then were placed again in a humidified atmosphere at 37° C. in 5% CO₂/95% air for 7 hr. Plate wells were gently washed with PBS, pH 7.4, and lysed with passive lysis buffer (1×, 100 μl, Promega). The subsequent chemiluminescent lysates were analyzed with the Luciferase Assay System (Promega) utilizing a TD-20/20 luminometer. The relative light units (photons) were determined by the addition of firefly luciferase substrate (75 μl) to cell lysate (10 μl). The light units were then normalized to the amount of protein in the well (mg/ml) with BCA™ Protein Assay Kit (Pierce) and standardized to percent of control (TNFα).

To determine cell viability, cells were treated as above but with 15 μM analog. After gently washing to remove any dead cells, they were given media (100 μl) and CellTiter 96® AQueous One Solution reagent (20 μl) for I hour and read at 490 nm with a Spectromax plate reader.

Curcumin is a known inhibitor of the NF-κB activation cascade. Therefore, modification of the structure of curcumin could lead to enhanced activity. The library consisting of three series of curcumin analogs were used to examine the role of the enone functionality in aryl systems where the spacer is 7-carbons (as in curcumin), 5-carbons or 3-carbons in length. In addition, the importance of aryl ring substituents was assessed. The NF-κB activities of curcumin and analogs were determined by a cellular firefly luciferase assay. This assay utilized a commercially available cell line (Panomics 293T-luc cellular assay) developed for screening inhibitors of NF-κB. This cell line is stably transfected with a luciferase reporter controlled by an NF-κB dependent promoter. The cell is stimulated with tumor necrosis factor alpha (TNFα) which activates NF-κB. NF-κB then binds to one of six promoter regions on the cell's DNA leading to the production of a luciferase enzyme. Luciferin is added to the cell lysates and the luciferase enzyme catalyzes a cleavage of luciferin leading to the emission of light.

The following FIGS. (4A-4C) show analogs active in the NF-κB cellular assay. The active analogs in FIGS. 4A-4C are arranged from highly active on the left to slightly active on the right.

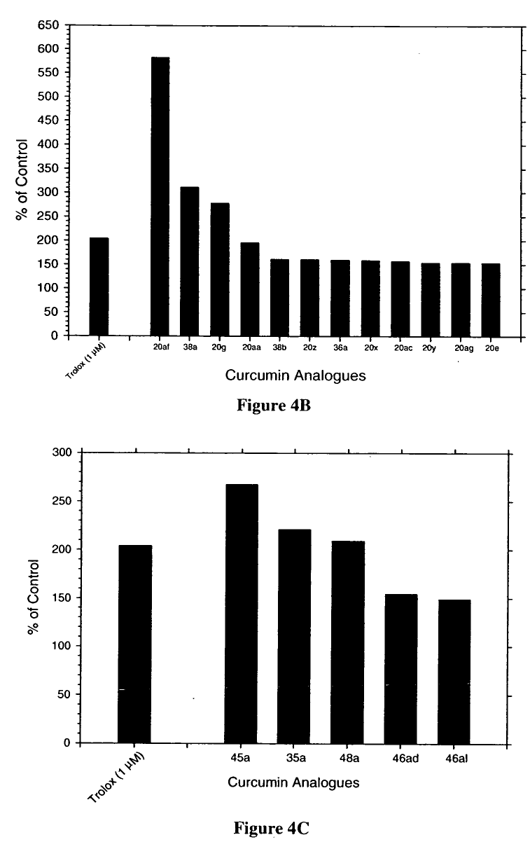

Active analogs in series l, which contain a 7-carbon spacer, are shown in FIG. 5A. Three analogs, 9a, 6a and 14a in this series were more active than curcumin (3a). These three analogs all contain the same aryl substituents as in curcumin. In addition, five of the six best analogs in this series contain a substituent on the central methylene carbon, indicating this position may be important to enhance activity. Three analogs also contain a saturated 7-carbon spacer indicating that saturation may be important in this series. Four of the six active analogs in this series have antioxidant activity. It is important to note that two analogs were active against NF-κB activation independent of antioxidant activity.

Active analogs in series 2, which contain a 5-carbon spacer, are shown in FIG. 5B. Ten analogs, 29, 38a, 20v, 31, 20a, 20ag, 20q, 20w, 20m and 20o in this series were more active than curcumin. Eight of the ten active analogs in this series contain aryl substituents. Six analogs contain substituents meta to the spacer on the aryl group, indicating this position may be important for NF-κB activity. Analogs 29 and 31 contain pyridine rings with no substituents on the ring. These two active analogs indicate that if the analogs in this series have a specific target, the target may contain a hydrogen bond donor in the area of binding. Only one of the ten active analogs in this series displays antioxidant activity. Therefore, it can then be concluded that these analogs are targeting a specific protein.

Active analogs in series 3, which contain a 3-carbon spacer, are shown in FIG. 5C. Three analogs, 45a, 52b and 35a, in this series were more active than curcumin. Three of the seven active analogs in this series retain the same aryl substituents as in curcumin. Analog 52b contains a piperidone ring on the spacer, indicating this type of spacer may be important for activity. Two of the seven active analogs were active as antioxidants. It is important to note that five analogs were active against NF-κB independent of antioxidant activity.

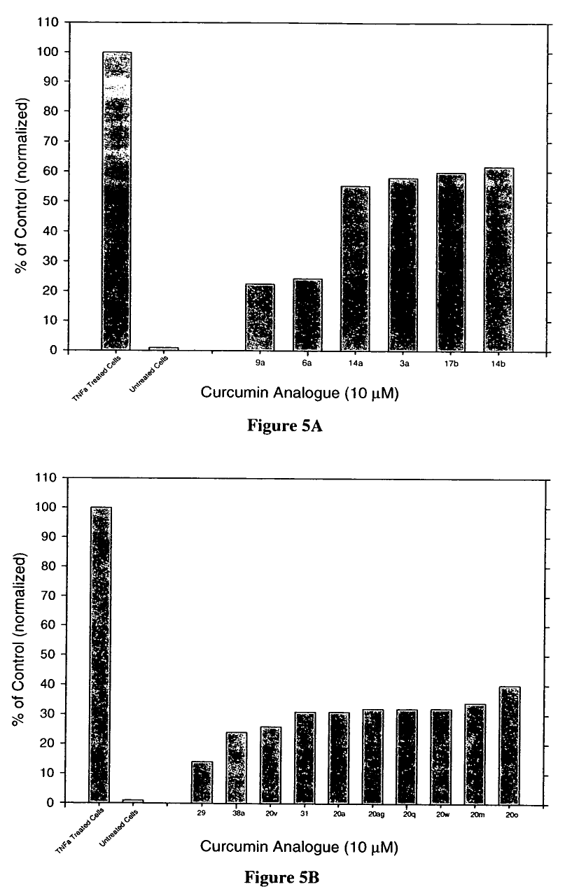

The IC₅₀ values for the active analogs against NF-κB were also measured. An IC₅₀ value is the concentration of the analog necessary to give 50% inhibition of NF-κB activation. The IC₅₀ plot for curcumin is shown in FIG. 6A. IC₅₀ plots for additional active analogs are shown in FIGS. 6B-6L. Table 4 shows IC₅₀ values for eight of the active analogs from the screening assay. Table 4 also shows if each analog was active as an antioxidant (+) in both the TRAP and FRAP assays. Of the IC₅₀ values obtained, curcumin (8.2 μM) is the least potent analog against NF-κB. Analogs 29 and 31 which contain pyridine rings are the most active analogs against NF-κB with IC₅₀ values of 3.5 and 3.4 μM. As observed in Table 4, five analogs are active against NF-κB independent of antioxidant activity. This indicates that the analogs are targeting specific proteins in the cell.

TABLE 4
IC₅₀ Values and Antioxidant Results for Active Analogs Against NF-κB.

| | Analog | IC₅₀ | | |
|---|---|---|---|---|
| Structure | Number | (μM) | TRAP | FRAP |
| 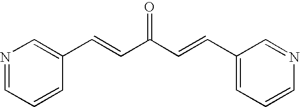 | 31 | 3.4 | − | − |
| 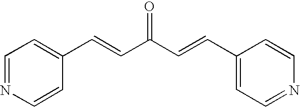 | 29 | 3.5 | − | − |
| 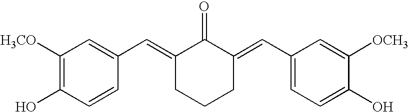 | 38a | 4.2 | + | + |
| 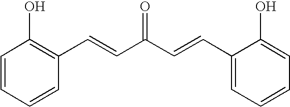 | 20q | 4.2 | + | − |
| 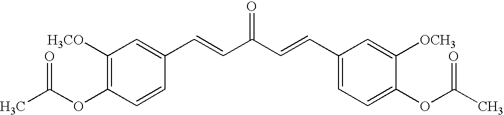 | 20ag | 5.4 | − | + |
| 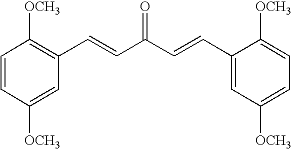 | 20m | 6.4 | − | − |
| 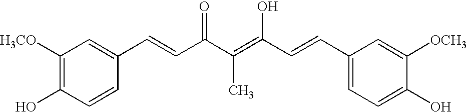 | 6a | 6.8 | + | + |
| 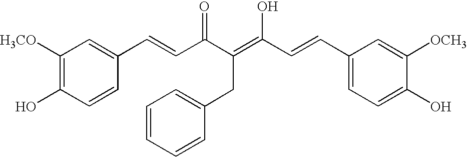 | 9a | 7.6 | + | + |

### Example 4

### Molecular Modeling of Curcumin Derivatives Binding to NF-κB

Molecular modeling studies can be performed to obtain useful information for the design of potent analogs. Modeling allows the visualization of ligand-protein interactions which can identify a potential inhibitor binding site in a protein. In most cases the substrate binding site is known from crystal structures that contain the native substrate or a substrate analog. Binding sites are also identified through crystal structure data involving bound inhibitors. Removal of the known inhibitor and addition of a potential inhibitor can give useful information concerning inhibitor protein interactions as well as estimated inhibition constants. Estimated inhibition constants (Kₑₛₚ) can be obtained from the docking studies with the modeling program. These constants can be compared to experimentally obtained inhibition constants (Kₑₓₚ). If a correlation between Kₑₛₜ and Kₑₓₚ is found then a new potential inhibitor can be docked to obtain Kₑₛₜ to determine if synthesis of the analog is warranted.

The dockings were performed using the docking program Autodock 3.0 (Morris et al., J. Comp. Chem. 1998, 19(14), 1639-1662; Morris et al., J. Comput.-Aided Mol. Des. 1996, 10(4), 293-304) on a cluster of Silicon Graphics workstations consisting of Octanes and O2s. The analogs, prepared using Sybyl 7.0 (Tripos Inc.), were drawn, assigned partial charges using the included Gasteiger-Hückel method and energy minimized using the Broyden, Fletcher, Goldfarb and Shanno (BFGS) optimization method. Minimizations were run for 10,000 iterations and all rotateable bonds were defined before docking. The proteins were prepared before docking in Sybyl by removing non-native substrates and water molecules. Polar hydrogens and Kollman Uni charges were added to the proteins as well. The molecules were docked in an area defined around the protein as a cube of either 60×60×60 Å or 120×120×120 Å.

Since the protein target of the analogs is unknown, molecular modeling was employed to examine a possible correlation between Kₑₛₜ and Kₑₓₚ. On proteins such as HSP90 (protein data bank code 1 YER and 1 YES) and glutathione S-transferase (19GS) the location of the analog binding site is known. Docking studies were performed using Autodock 3.0 and the resulting Kₑₛₜ was compared to Kₑₓₚ.

On proteins such as NF-κB (1IKN and 1SCV) and AP-1 (1FOS), the location of analog binding site is not known. Therefore, it was necessary to identify any and all potential binding areas and model the analogs to these areas. Fortunately, a program has been developed that can identify binding areas (Brown et al., J. Chem. Inf. Comp. Sci. 2004, 44(4), 1412-1422). The program called the Macromolecule Encapsulating Surface (MES) program generates a flexible surface over the entire protein and determines how much unoccupied volume there is between the generated surface and the surface of the protein. If there is a large space, that area is a potential binding site and it is possible for a potential inhibitor to fill this space. On the other hand, if there is no space the program overlooks that area and dismisses it from future consideration. Once all of the potential binding areas are identified, the program will dock inhibitors in each of these locations and determine the Kₑₛₜ for each analog.

Binding to NF-κB

When performing docking studies of the potential analogs against NF-κB, two crystal structures, 1IKN and 1SVC, were selected from the twelve available in the protein databank. These two crystal structures were selected because one (1IKN) contained both the p50 and p65 subunits of NF-κB. The other crystal structure was selected because it contained the p50 subunit of NF-κB bound to a short segment of DNA.

Binding to the 1IKN Form of NF-kB

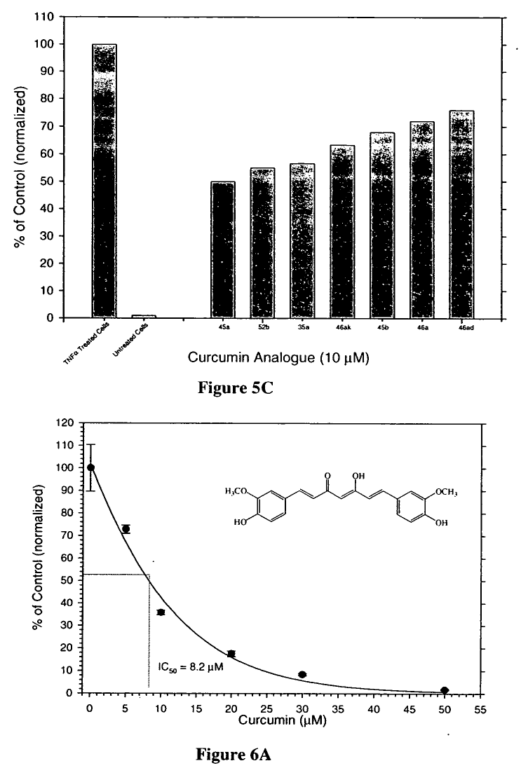

1IKN (Huxford et al., Cell 1998, 95, 759-770) was selected because it contains both the p50 and p65 subunits of NF-κB, the most common heterodimer. The p50 subunit was not the complete subunit. A second reason 1IKN was selected was because it contained I-κB, the natural inhibitor of NF-κB. Since I-κB is phosphorylated at serine residues 32 and 36, in the activation cascade of NF-κB, it was hoped that the crystal structure would contain these residues to see if the potential analogs blocked them from being phosphorylated. Unfortunately, the crystal structure of I-κB did not contain these residues and thus docking studies could not be performed directly on the I-κB subunit. FIG. 7 shows the p50 and p65 NF-κB heterodimer complexed to I-κB. In FIG. 7, the blue protein is the p50 subunit, the red protein is the p65 subunit and the yellow protein is I-κB.

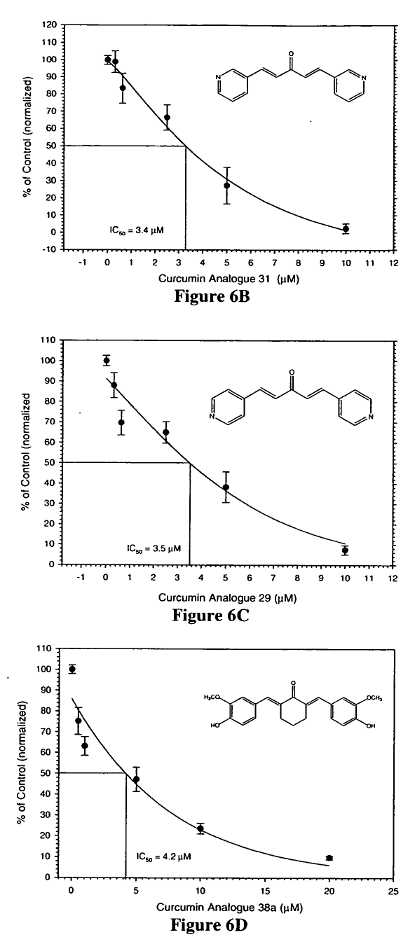

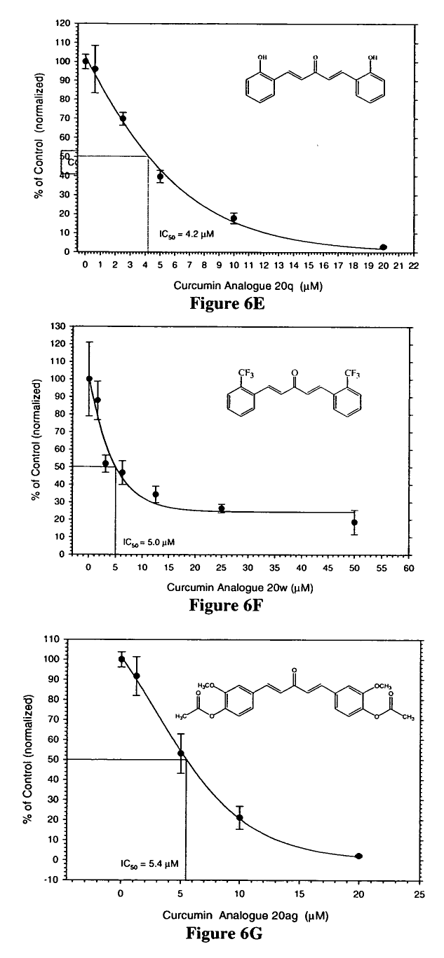

When I-κB is removed as shown in FIG. 8, a new face of the heterodimer is revealed. It is believed that DNA binds to this new face of the protein after NF-κB translocates to the nucleus. If the potential analogs inhibit NF-κB from binding to DNA and thus stopping transcription from occurring, then the potential analogs should bind to this face of the molecule. However, only analogs 9a, 9b, 12b, 15a, 15b, 17b, 20l and 52l of the analog library bind to the newly exposed face as shown in FIG. 9. Analogs 12b, 15a and 17b have a good Kₑₛₜ values and rank in the top nine analogs. Analog 12b binds to NF-κB on this newly exposed face of the molecule and the Kₑₛₜ value is good at 2.00E-10 M. These results indicate the analogs should be blocking the NF-κB-DNA interaction. However, there is no correlation between the analogs that bind on this face of the molecule and the experimental results of these analogs. Based on the docking studies, it does not appear that the analogs block a NF-κB-DNA interaction, but it is possible that they inhibit NF-κB in another manner.

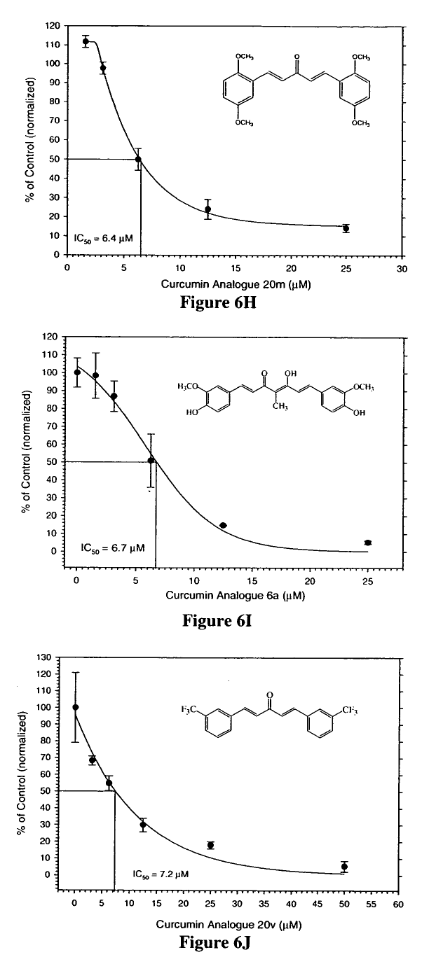

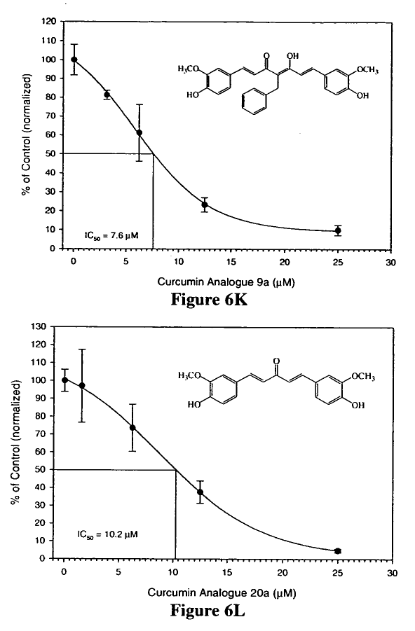

Curcumin (3a), shown in FIG. 10, and the other potential inhibitors bind on the opposite side of the molecule as shown in FIG. 11. Many of the analogs that bind on the opposite side also have good Kₑₛₜ values with analogs 3i, 20ag, 23 and 53 being the best. Table 5 shows each analog with its Kₑₛₜ value in molar units. Again, there is no correlation between Kₑₛₜ and Kₑₓₚ. This indicates that the potential inhibitors probably do not bind to the NF-κB heterodimer.

TABLE 5
Kₑₛₜ Values of Curcumin Analogs Against NF-κB Without MES.

| | |
|---|---|
| 12b | 2.00E−10 |
| 20ag | 3.07E−10 |
| 17b | 3.27E−10 |
| 53 | 3.72E−10 |
| 3i | 6.31E−10 |
| 23 | 7.36E−10 |
| 20n | 7.86E−10 |
| 25 | 1.16E−09 |
| 15a | 1.20E−09 |
| 3d | 1.34E−09 |
| 14a | 1.34E−09 |
| 9a | 1.51E−09 |
| 3b | 1.94E−09 |
| 3g | 2.32E−09 |
| 38a | 2.36E−09 |
| 14b | 2.38E−09 |
| 20m | 2.49E−09 |
| 15b | 2.89E−09 |
| 3a | 2.94E−09 |
| 6b | 3.06E−09 |
| 13a | 3.17E−09 |
| 20i | 3.29E−09 |
| 11b | 3.31E−09 |
| 20v | 3.38E−09 |
| 52e | 3.52E−09 |
| 9b | 4.09E−09 |
| 36a | 4.55E−09 |
| 6a | 4.62E−09 |
| 20w | 5.08E−09 |
| 20o | 5.32E−09 |
| 20a | 5.42E−09 |
| 20p | 5.42E−09 |
| 16b | 5.71E−09 |
| 20k | 6.38E−09 |
| 52aa | 6.52E−09 |
| 52ac | 7.38E−09 |
| 3f | 7.60E−09 |
| 20d | 7.74E−09 |
| 20c | 7.85E−09 |
| 3h | 8.02E−09 |
| 46ad | 8.96E−09 |
| 20y | 9.03E−09 |
| 20u | 9.53E−09 |
| 20q | 9.59E−09 |
| 20ah | 9.80E−09 |
| 20z | 1.11E−08 |
| 45a | 1.20E−08 |
| 40af | 1.29E−08 |
| 20x | 1.35E−08 |
| 3c | 1.39E−08 |
| 20ab | 1.63E−08 |
| 13b | 1.68E−08 |
| 20ae | 1.75E−08 |
| 52b | 1.76E−08 |
| 38b | 1.87E−08 |
| 48a | 1.92E−08 |
| 20t | 2.35E−08 |
| 36e | 2.39E−08 |
| 20g | 2.45E−08 |
| 3e | 2.54E−08 |
| 20e | 2.60E−08 |
| 48ad | 2.71E−08 |
| 20aa | 2.85E−08 |
| 20ac | 2.90E−08 |
| 46a | 3.07E−08 |
| 20r | 3.16E−08 |
| 31 | 3.66E−08 |
| 29 | 4.87E−08 |
| 46al | 5.03E−08 |
| 46ak | 5.16E−08 |
| 39b | 5.32E−08 |
| 20b | 5.82E−08 |
| 20s | 6.08E−08 |
| 20f | 6.66E−08 |
| 42b | 6.78E−08 |
| 20l | 7.00E−08 |
| 50b | 7.12E−08 |
| 34 | 9.37E−08 |
| 40b | 1.23E−07 |
| 43b | 1.41E−07 |
| 52l | 1.76E−07 |
| 45b | 2.19E−07 |
| 35a | 5.30E−07 |
| 35e | 1.66E−06 |
| 35q | 3.53E−06 |

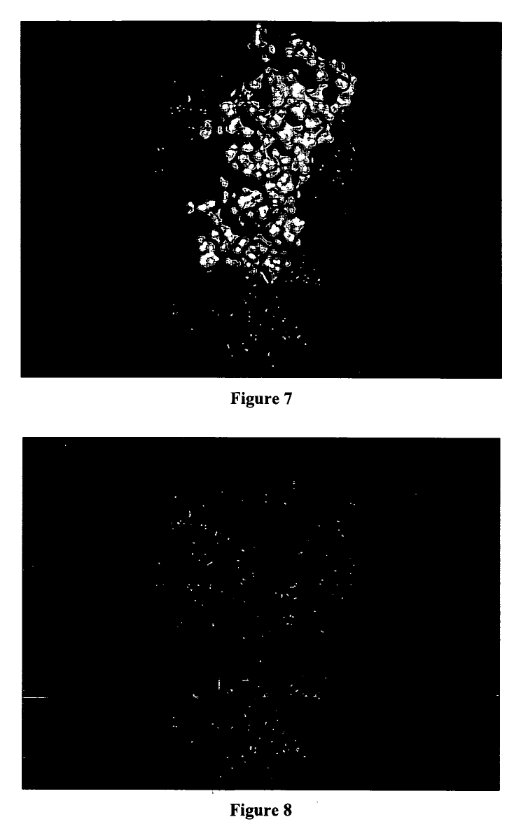

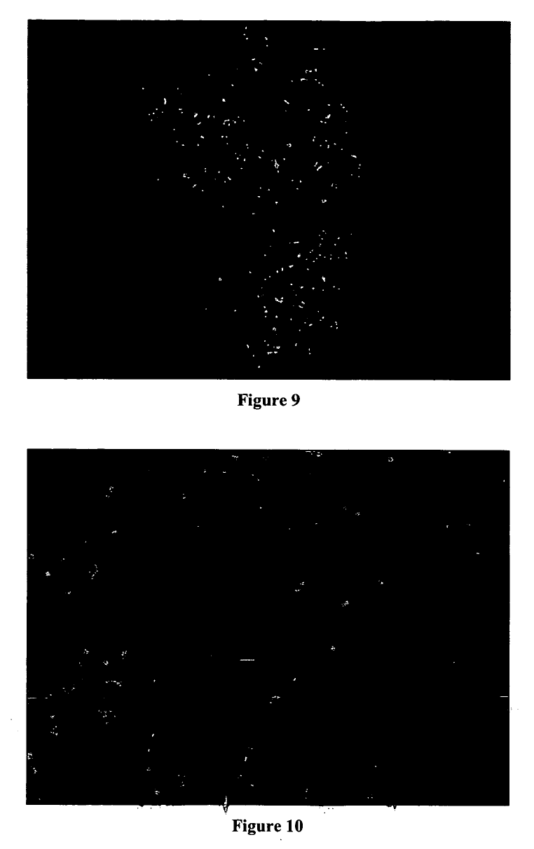

To verify these findings, the MES program was utilized on the NF-κB heterodimer to identify any potential binding areas for the analogs. The results of this docking study are different than the docking results obtained without the use of the MES program. With the MES program, all the potential inhibitors bind on the new face of the NF-κB heterodimer as shown in FIG. 12. The visual results of this docking study indicate that the analogs should be good inhibitors of NF-κB and in particular of a NF-κB-DNA interaction. Most of the potential inhibitors bind to NF-κB with good Kₑₛₜ values with analogs 9a, 12b, 13a, 15a and 20ag showing the best activity as shown in Table 6. Curcumin (3a), as shown in FIG. 13, binds to the heterodimer towards the bottom portion of the p50 subunit and has a Kₑₛₜ value of 9.64E-8 M. However, the Kₑₛₜ values of curcumin and its analogs do not correlate to Kₑₓₚ values, they probably do not bind to NF-κB.

TABLE 6
Kₑₛₜ Values for NF-κB (1IKN) with MES.

| | |
|---|---|
| 20ag | 7.26E−09 |
| 9a | 7.39E−09 |
| 15a | 1.26E−08 |
| 12b | 1.54E−08 |
| 13a | 1.57E−08 |
| 17b | 1.65E−08 |
| 9b | 1.93E−08 |
| 25 | 2.51E−08 |
| 3d | 2.59E−08 |
| 20ae | 2.65E−08 |
| 20i | 2.69E−08 |
| 38a | 3.27E−08 |
| 20a | 4.38E−08 |
| 20ah | 5.27E−08 |
| 20m | 5.61E−08 |
| 53 | 6.06E−08 |
| 14a | 6.10E−08 |
| 20v | 6.66E−08 |
| 23 | 7.21E−08 |
| 11b | 7.35E−08 |
| 20n | 8.21E−08 |
| 6a | 8.63E−08 |
| 40af | 8.72E−08 |
| 14b | 8.86E−08 |
| 20k | 9.43E−08 |
| 3a | 9.64E−08 |
| 15b | 9.69E−08 |
| 20u | 9.88E−08 |
| 36a | 1.06E−07 |
| 20w | 1.29E−07 |
| 3h | 1.40E−07 |
| 3g | 1.47E−07 |
| 46ad | 1.56E−07 |
| 20o | 1.63E−07 |
| 20f | 1.74E−07 |
| 3i | 1.82E−07 |
| 16b | 1.82E−07 |
| 36e | 1.86E−07 |
| 20d | 1.86E−07 |
| 20g | 1.86E−07 |
| 20e | 1.87E−07 |
| 13b | 2.03E−07 |
| 45a | 2.05E−07 |
| 6b | 2.19E−07 |
| 20y | 2.22E−07 |
| 13c | 2.41E−07 |
| 52e | 2.47E−07 |
| 20p | 2.56E−07 |
| 52l | 2.57E−07 |
| 20aa | 2.71E−07 |
| 3e | 2.74E−07 |
| 20x | 2.81E−07 |
| 20ab | 2.82E−07 |
| 20z | 3.78E−07 |
| 52aa | 3.84E−07 |
| 3b | 4.03E−07 |
| 20q | 4.16E−07 |
| 20c | 4.46E−07 |
| 3f | 4.47E−07 |
| 52ac | 4.61E−07 |
| 20ac | 4.92E−07 |
| 20s | 5.49E−07 |
| 20r | 6.43E−07 |
| 48a | 6.44E−07 |
| 48ad | 7.60E−07 |
| 20t | 8.03E−07 |
| 20l | 8.15E−07 |
| 50b | 8.72E−07 |
| 46ak | 9.22E−07 |
| 42b | 9.44E−07 |
| 40b | 9.64E−07 |
| 29 | 1.03E−06 |
| 52b | 1.08E−06 |
| 46al | 1.14E−06 |
| 46a | 1.24E−06 |
| 20b | 1.25E−06 |
| 31 | 1.33E−06 |
| 39b | 1.42E−06 |
| 43b | 2.01E−06 |
| 38b | 2.41E−06 |
| 45b | 4.15E−06 |
| 34 | 4.22E−06 |
| 35a | 4.46E−06 |
| 35e | 4.73E−06 |
| 35q | 4.81E−06 |

Binding to the 1SVC Form of NF-κK

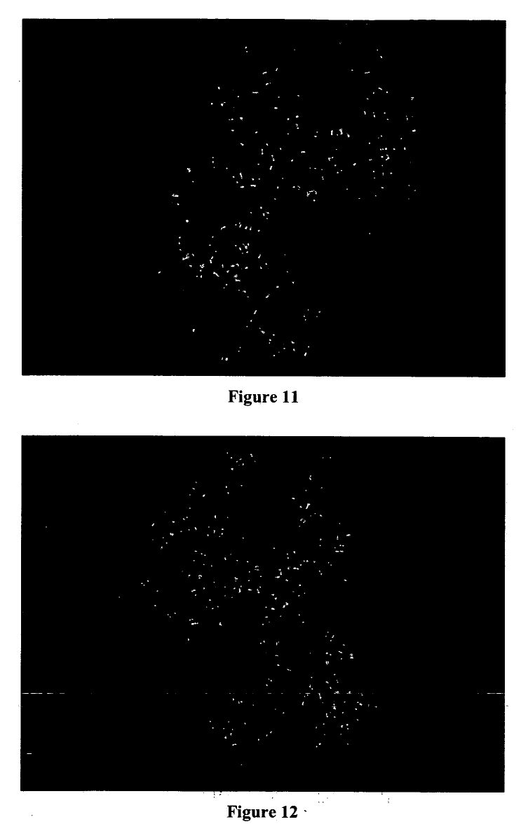

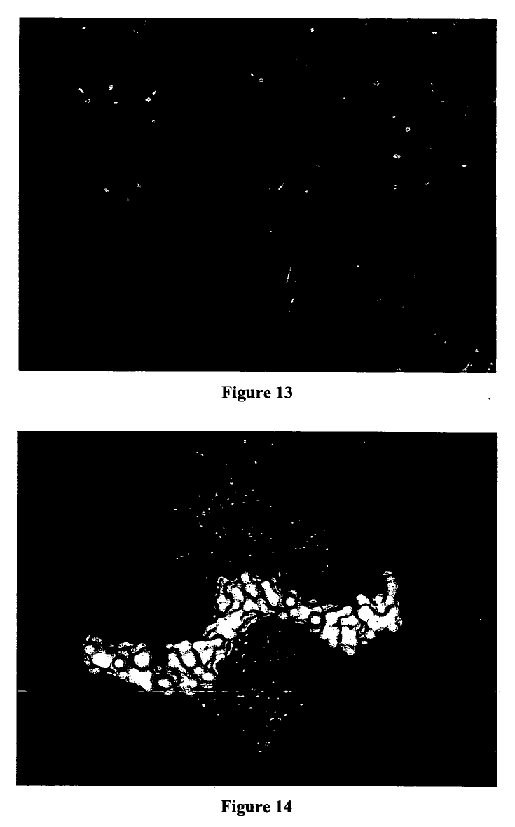

1SVC (Mueller et al., Nature, 1995, 373, 311-317) was selected because it contains the p50 subunit of NF-κB bound to a small portion of DNA. This crystal structure was important because it contained almost all of the p50 subunit and because it had the exact site of DNA binding. This would provide additional information concerning the blocking of NF-κB-DNA binding interactions of the analogs. FIG. 14 shows the p50 subunit of NF-κB bound to a small portion of DNA. In FIG. 14, the blue protein is the p50 subunit and the yellow segment is the DNA. When the DNA is removed as shown in FIG. 15, a new area is exposed. It is in this location that the analogs will bind if they are preventing a NF-κB-DNA binding interaction.

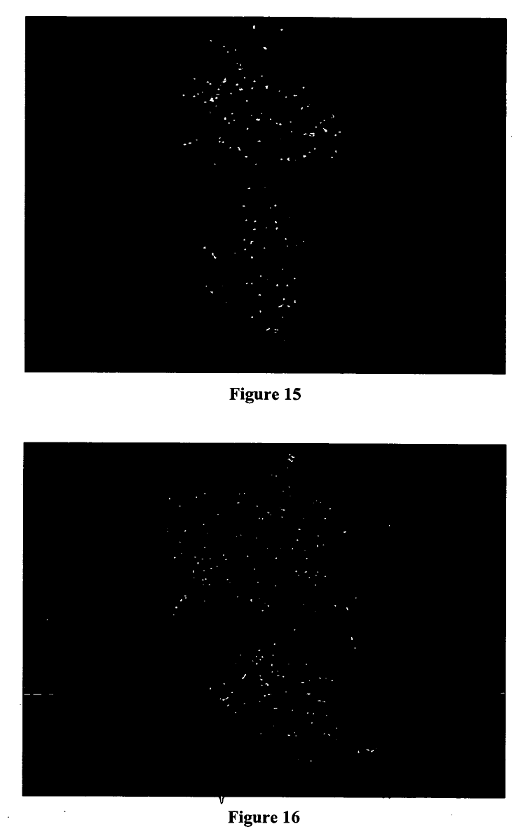

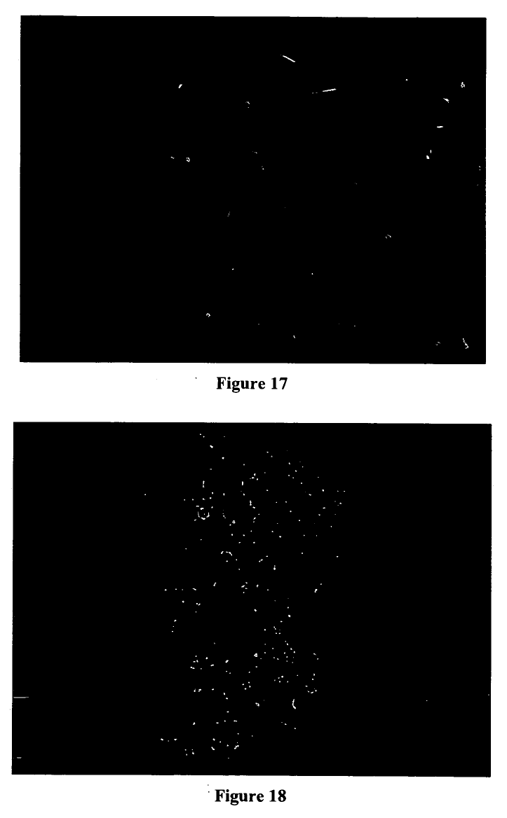

When docking studies were performed, most of the potential inhibitors bind in the general area the DNA once occupied as shown in FIG. 16. It is apparent that in the location of binding, there is a “small hole” to which all of the analogs on this portion of the molecule bind. FIG. 17 shows curcumin (3a) bound in the “small hole”.

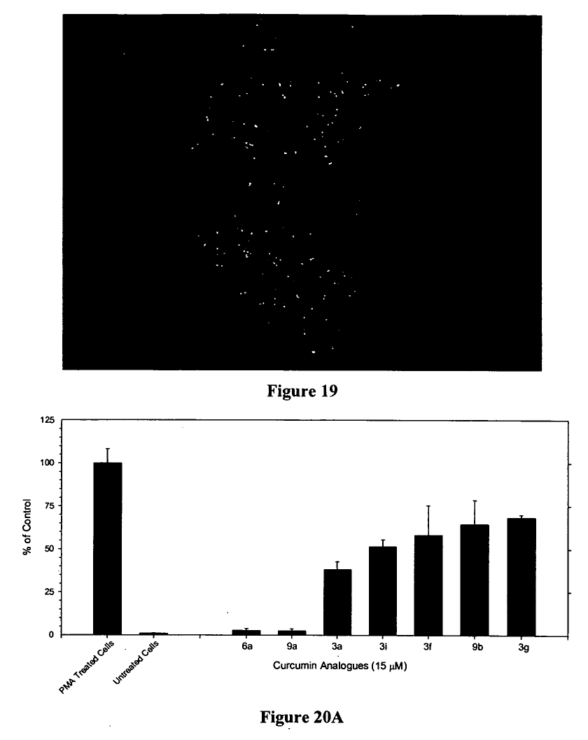

Many of these analogs bind with good Kₑₛₜ values with analog 9b displaying the best inhibitory activity at 3.79E-10 M as shown in Table 7. Based on these Kₑₛₜ values, several analogs should inhibit the blocking of NF-κB-DNA binding interactions. However, there is no correlation to the Kₑₓₚ results indicating that these analogs probably do not inhibit this type of an interaction. It is possible that there could be another mode of action that potential inhibitors could be displaying since seven analogs bind on the opposite side of the protein as shown in FIG. 18. These seven analogs, 12b, 15a, 15b, 52e, 521, 52aa and 52ac have rather poor Kₑₛₜ values with the exception of analog 12b which was ranked as the third best potential inhibitor. Since these analogs have poor Kₑₛₜ values and there is no correlation to any Kₑₓₚ results, they are likely not inhibitors of the NF-κB protein.

TABLE 7
Kₑₛₜ Values for NF-κB (1SVC).

| | |
|---|---|
| 9b | 3.79E−10 |
| 3g | 1.59E−09 |
| 12b | 3.30E−09 |
| 25 | 4.39E−09 |
| 6b | 4.40E−09 |
| 3i | 5.53E−09 |
| 6a | 5.62E−09 |
| 23 | 5.67E−09 |
| 20e | 6.09E−09 |
| 3e | 6.84E−09 |
| 20m | 7.34E−09 |
| 3d | 7.35E−09 |
| 14b | 7.35E−09 |
| 13a | 8.18E−09 |
| 3h | 9.21E−09 |
| 14a | 9.59E−09 |
| 20ah | 9.85E−09 |
| 20d | 1.07E−08 |
| 20v | 1.11E−08 |
| 20g | 1.19E−08 |
| 3b | 1.30E−08 |
| 20ae | 1.31E−08 |
| 20ag | 1.32E−08 |
| 20u | 1.33E−08 |
| 20o | 1.34E−08 |
| 20x | 1.42E−08 |
| 11b | 1.47E−08 |
| 3a | 1.57E−08 |
| 36a | 1.75E−08 |
| 20a | 1.87E−08 |
| 13c | 1.96E−08 |
| 13b | 1.97E−08 |
| 20aa | 2.12E−08 |
| 20y | 2.22E−08 |
| 20q | 2.78E−08 |
| 16b | 2.83E−08 |
| 17b | 2.95E−08 |
| 3f | 3.00E−08 |
| 36e | 3.34E−08 |
| 20f | 3.41E−08 |
| 20ab | 3.51E−08 |
| 20i | 4.10E−08 |
| 20k | 4.55E−08 |
| 20w | 4.68E−08 |
| 20ac | 4.70E−08 |
| 53 | 4.70E−08 |
| 15a | 4.75E−08 |
| 20t | 5.41E−08 |
| 20r | 6.15E−08 |
| 20l | 6.62E−08 |
| 20n | 7.54E−08 |
| 20p | 7.64E−08 |
| 46ad | 7.96E−08 |
| 46al | 8.38E−08 |
| 20c | 8.47E−08 |
| 42b | 9.25E−08 |
| 20s | 9.78E−08 |
| 31 | 1.11E−07 |
| 20z | 1.13E−07 |
| 9a | 1.18E−07 |
| 40af | 1.21E−07 |
| 15b | 1.27E−07 |
| 48a | 1.45E−07 |
| 39b | 1.55E−07 |
| 20b | 1.57E−07 |
| 29 | 1.65E−07 |
| 48ad | 1.85E−07 |
| 40b | 1.86E−07 |
| 38a | 1.87E−07 |
| 46a | 1.94E−07 |
| 50b | 1.94E−07 |
| 45a | 2.03E−07 |
| 34 | 2.89E−07 |
| 46ak | 2.96E−07 |
| 52aa | 3.43E−07 |
| 38b | 4.41E−07 |
| 52e | 4.42E−07 |
| 45b | 5.15E−07 |
| 43b | 5.65E−07 |
| 52l | 5.67E−07 |
| 52ac | 6.59E−07 |
| 35q | 9.89E−07 |
| 52b | 1.05E−06 |
| 35a | 1.07E−06 |
| 35e | 1.18E−06 |

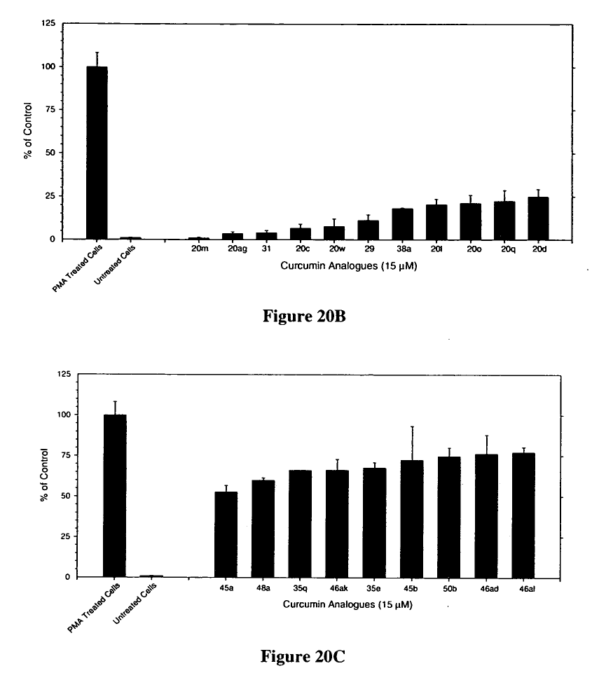

To verify these findings, the MES program was utilized on the p50 subunit of NF-κB to identify any potential binding areas for the analogs. The results of this docking study are slightly different than those when the MES program was not used. All the potential inhibitors bind to an area directly below the DNA binding area and wrap around to the backside of the protein, as shown in FIG. 19, indicating they may inhibit the NF-κB-DNA interaction. None of the potential inhibitors bind in the “small hole” as in the docking results without the MES program (FIG. 17). The Kₑₛₜ values for these analogs are mediocre, with the best analog, 15a, having a Kₑₛₜ of 2.27E-08 M as shown in Table 8 in the appendix. Since the library of analogs does not display good Kₑₛₜ values or correlate with any Kₑₓₚ results, NF-κB does not appear to be the target for curcumin analogs.

TABLE 8
Kₑₛₜ Values for NF-κB (1SVC) with MES.

| | |
|---|---|
| 15a | 2.27E−08 |
| 17b | 2.48E−08 |
| 12b | 2.63E−08 |
| 20ag | 5.48E−08 |
| 15b | 6.30E−08 |
| 38a | 7.76E−08 |
| 9a | 8.10E−08 |
| 6a | 8.37E−08 |
| 53 | 9.53E−08 |
| 3h | 1.01E−07 |
| 3d | 1.29E−07 |
| 9b | 1.32E−07 |
| 23 | 1.58E−07 |
| 25 | 2.36E−07 |
| 3i | 2.67E−07 |
| 14a | 2.78E−07 |
| 20p | 3.01E−07 |
| 13a | 3.13E−07 |
| 20i | 3.22E−07 |
| 16b | 3.31E−07 |
| 20m | 3.58E−07 |
| 20v | 4.08E−07 |
| 11b | 4.15E−07 |
| 3f | 4.22E−07 |
| 40af | 4.26E−07 |
| 3g | 4.37E−07 |
| 3e | 4.45E−07 |
| 20ah | 4.51E−07 |
| 3a | 4.79E−07 |
| 20u | 4.82E−07 |
| 20ae | 5.00E−07 |
| 13c | 5.36E−07 |
| 20o | 5.47E−07 |
| 14b | 5.50E−07 |
| 20w | 5.71E−07 |
| 20a | 5.80E−07 |
| 20n | 6.46E−07 |
| 46ad | 6.58E−07 |
| 36a | 6.79E−07 |
| 46a | 6.81E−07 |
| 13b | 7.62E−07 |
| 6b | 7.66E−07 |
| 45a | 8.30E−07 |
| 20l | 8.98E−07 |
| 3b | 9.38E−07 |
| 20d | 1.01E−06 |
| 20k | 1.19E−06 |
| 52e | 1.21E−06 |
| 20e | 1.22E−06 |
| 20f | 1.27E−06 |
| 20g | 1.28E−06 |
| 20ab | 1.34E−06 |
| 20y | 1.34E−06 |
| 52l | 1.46E−06 |
| 20aa | 1.47E−06 |
| 20x | 1.55E−06 |
| 20z | 1.58E−06 |
| 20c | 1.70E−06 |
| 20q | 1.74E−06 |
| 20r | 1.77E−06 |
| 46al | 1.81E−06 |
| 29 | 1.95E−06 |
| 38b | 2.02E−06 |
| 48a | 2.06E−06 |
| 20ac | 2.12E−06 |
| 40b | 2.13E−06 |
| 48ad | 2.31E−06 |
| 52aa | 2.40E−06 |
| 31 | 2.95E−06 |
| 20s | 2.98E−06 |
| 39b | 3.04E−06 |
| 46ak | 3.31E−06 |
| 20t | 3.52E−06 |
| 36e | 3.67E−06 |
| 20b | 4.27E−06 |
| 52ac | 4.50E−06 |
| 42b | 6.29E−06 |
| 50b | 6.32E−06 |
| 52b | 7.86E−06 |
| 35q | 8.52E−06 |
| 45b | 9.20E−06 |
| 35a | 1.11E−05 |
| 35e | 1.30E−05 |
| 34 | 1.84E−05 |
| 43b | 1.88E−05 |

### Example 5

### Inhibition of AP-1 Activity by Curcumin Derivatives

Curcumin and its analogues were screened for activity against AP-1 by a cellular assay using the AP-1 stable cell line (293AP1-luc). The cell line is derived from human 293 embryonic kidney cells containing a chromosomal integration of a luciferase reporter construct regulated by 3 copies of the AP-1 response element (Panomics, Inc.). This cell line is obtained by co-transfection of pAP1-luc and pTK-hyg containing plasmids followed by the addition of hygromycin (200 μg/ml) to maintain cell selection.

The cell line was grown in a humidified atmosphere at 37° C. in 5% CO₂/95% air and maintained in Dulbecco's Modified Eagle's Medium (DMEM-high glucose containing 4 mM glutamine) containing fetal bovine serum (FBS, 10%), sodium pyruvate (1 mM), penicillin (100 units/ml), streptomycin (100 μg/ml) and hygromycin (100 μg/ml) to maintain cell selection (Gibco/Invitrogen).

The 293/AP1-luc cells were re-plated, 24 hr prior to treatment into, 24-well cell culture plates in media without hygromycin, to prevent it from interfering with the assay. The cells were then allowed to grow and attach, to the wells, for 24 hr in a humidified atmosphere at 37° C. in 5% CO₂/95% air. After 24 hr, the cells had reached approximately 60% confluency. The cells were then given media (1 ml) with or without phorbol 12-myristate 13-acetate (PMA, 10 ng/ml, Calciochem) followed by immediate treatments with curcumin or analogue (15 μM in DMSO). The cells were placed again in a humidified atmosphere at 37° C. in 5% CO₂/95% air for 24 hr. Plate wells were gently washed with PBS, pH 7.4, and lysed with passive lysis buffer (1×, 100 μl, Promega). The subsequent chemiluminescent lysates were analyzed with the Luciferase Assay System (Promega) utilizing a TD-20/20 luminometer. The relative light units (photons) were determined by the addition of firefly luciferase substrate (75 μl) to cell lysate (10 μl). The light units were then normalized to the amount of protein in the well (mg/ml) with BCA™ Protein Assay Kit (Pierce) and standardized to percent of control (PMA).

To determine cell viability, cells were treated as above but with 15 μM analogue. After gently washing to remove any dead cells, they were given media (100 μl) and CellTiter 96® AQueous One Solution reagent (20 μl) for 1 hour and read at 490 nm with a Spectromax plate reader.

Curcumin is a known inhibitor of the AP-1 activation cascade. Therefore, modification of the structure of curcumin could lead to analogs with enhanced activity. The library consisting of three series of curcumin analogs were used to examine the role of the enone functionality in aryl systems where the spacer is 7-carbons (as in curcumin), 5-carbons or 3-carbons in length. In addition, the importance of aryl ring substituents was assessed. The AP-1 activities of curcumin and analogs were determined by a cellular firefly luciferase assay. This assay utilized a commercially available cell line (Panomics 293-luc cellular assay) developed for screening inhibitors of AP-1. This cell line is stably transfected with a luciferase reporter controlled by an AP-1 dependent promoter. The cell is stimulated with phorbol ester which activates AP-1. AP-1 then binds to one of three promoter regions on the cells DNA leading to the production of a luciferase enzyme. Luciferin is added to the cell lysates and the luciferase enzyme catalyzes a cleavage of luciferin leading to the emission of light.

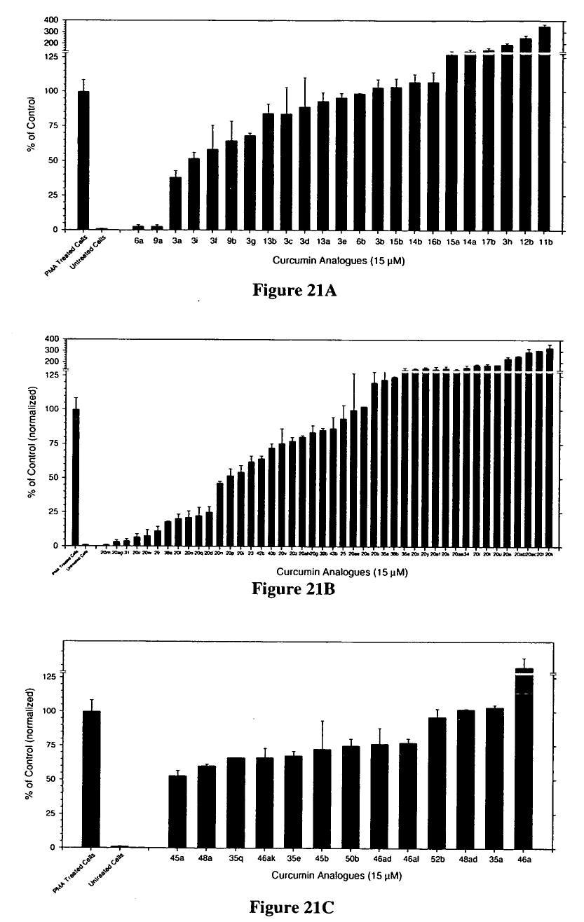

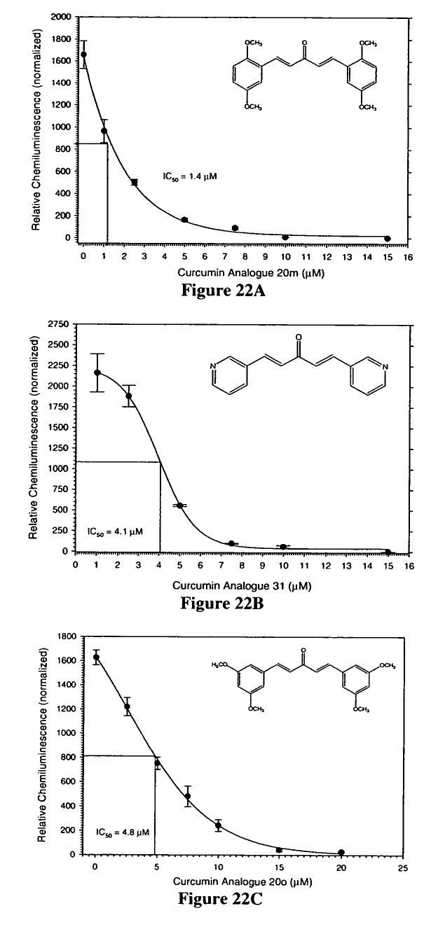

FIGS. 20A-C show analogs active in the AP-1 cellular assay. The active analogs in FIGS. 20A-C are arranged from highly active on the left to slightly active on the right. Figures containing all analogs can be found in FIGS. 21A-C.

Active analogs in series 1, which contain a 7-carbon spacer, are shown in FIG. 20A. Two analogs, 6a and 9a, in this series were more active than curcumin (3a). Both of these analogs contain the same aryl ring substituents as curcumin in addition to either a methyl (6a) or benzyl (9a) substituent on the central methylene carbon. A third active analog, 9b, also contains a central methylene benzyl substituent. No active analogs in this series contained a saturated spacer between the aryl groups. Four of the seven analogs in this series display activity in both antioxidant assays. It is important to note that three analogs were active against AP-1 independent of antioxidant activity.

Active analogs in series 2, which contain a 5-carbon spacer, are shown in FIG. 20B. Eleven analogs, 20m, 20ag, 31, 20c, 20w, 29, 38a, 20l, 20o, 20q and 20d, in this series were more active than curcumin. Of these eleven active analogs, nine contain substituted aryl groups. Six analogs contain substituents ortho to the spacer on the aryl group, indicating this position may be important for AP-1 activity. Analogs 29 and 31 contain pyridine rings with no substituents on the ring. These two active analogs indicate that if the analogs in this series have a specific target, the target may contain a hydrogen bond donor in the area of binding. Since only three of the eleven active analogs in this series display antioxidant activities, it is suggested that these analogs are targeting a specific protein.

Active analogs in series 3, which contain a 3-carbon spacer, are shown in FIG. 20C. No analog in this series was more active than curcumin. The active analogs in this series also exhibit good antioxidant activities. The two most active analogs, 45a and 48a, in this series displayed antioxidant activity in both antioxidant assays. Active analogs, 35q, 46ad and 46al, in this series were also active in one or the other antioxidant assay.

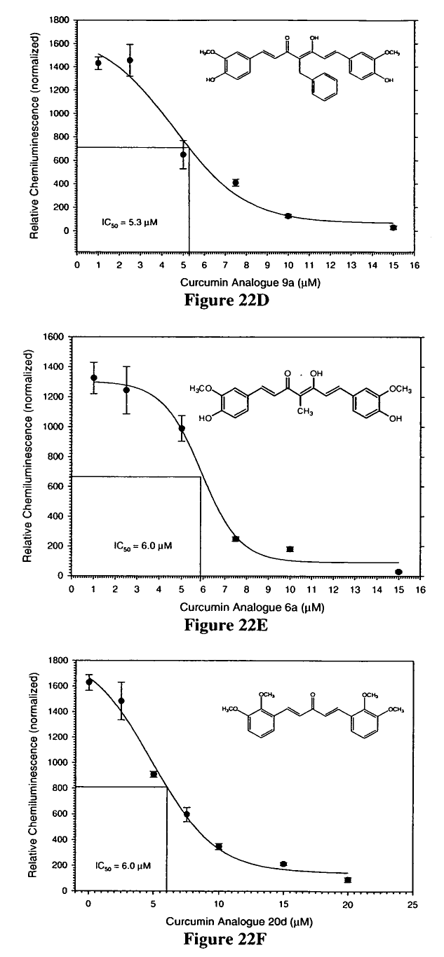

The IC₅₀ values for the twelve active analogs as well as curcumin against AP-1 were also measured. IC₅₀ plots for these active analogs are shown in FIGS. 22A-L. Of the twelve best analogs against AP-1, nine of the analogs also ranked in the top twelve against NF-κB activity. Table 9 shows the IC₅₀ values of the nine analogs that were active against both NF-κB and AP-1. The Table 10 also shows whether each analog was active as an antioxidant (+) in both the TRAP and FRAP assays.

TABLE 9
IC₅₀ Values and Antioxidant Results for Active Analogs Against NF-κB and AP-1.

| | | | NF- | | |
|---|---|---|---|---|---|
| | | AP-1 | κB | | |
| | Analog | IC₅₀ | IC₅₀ | | |
| Structure | Number | (μM) | (μM) | TRAP | FRAP |
| 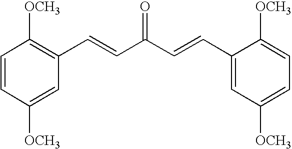 | 20m | 1.4 | 6.4 | − | − |
| 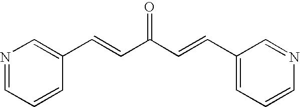 | 31 | 4.1 | 3.4 | − | − |
| 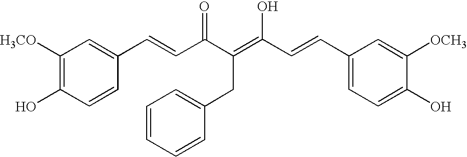 | 9a | 5.3 | 7.6 | + | + |
| 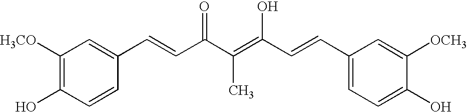 | 6a | 6.0 | 6.7 | + | + |
| 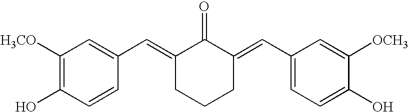 | 38a | 7.3 | 4.2 | + | + |
| 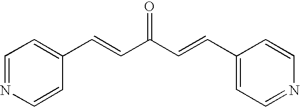 | 29 | 8.2 | 3.5 | − | − |
| 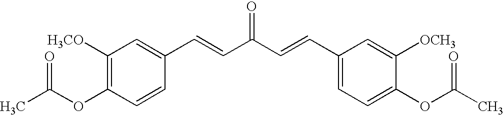 | 20ag | 8.3 | 5.4 | − | + |
| 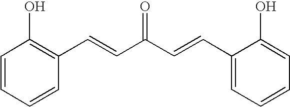 | 20q | 11.7 | 4.2 | + | − |
| 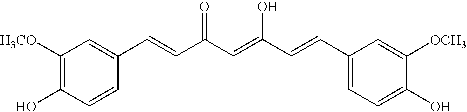 | 3a | 12.8 | 8.2 | + | + |

Of the IC₅₀ values obtained, curcumin (12.8 μM) is the least potent analog against AP-1. Analog 20m which has an ortho substituent is the most active analog against AP-1 with an IC50 value of 1.4 μM. As observed in Table 9, several analogs are active against AP-1 independent of antioxidant activity. This indicates that the analogs are targeting specific proteins in the cell. Since nine of the twelve best analogs against AP-1 are also active against NF-κB it is possible that these analogs are acting on a common target involved in both activation cascades and that the analogs are not inhibiting the AP-1 or NF-κB proteins directly.

### Example 6

### Molecular Modeling of Curcumin Derivatives binding to AP-1

When performing docking studies of the potential inhibitors against AP-1, one crystal structure (1FOS) was selected from the twenty five selections that were available. 1FOS (Glover et al., Nature 1995, 373, 257-261) was selected because it contained the c-Jun and c-Fos heterodimer, the most common heterodimer, complexed to a segment of DNA. This crystal structure was important because it contained the exact binding site of DNA to this heterodimer. This provided information concerning the blocking of AP-1-DNA binding interactions by the analogs.

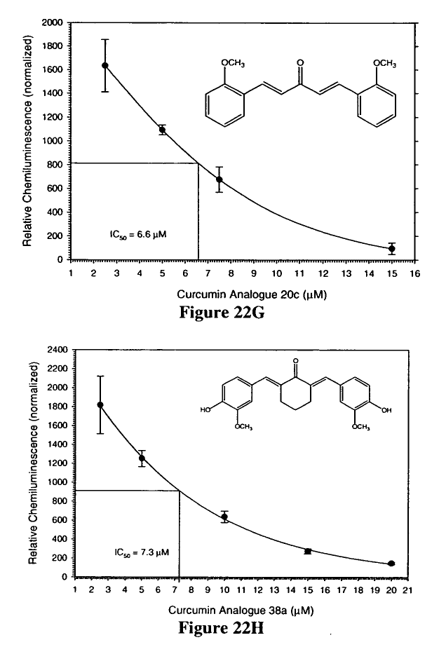

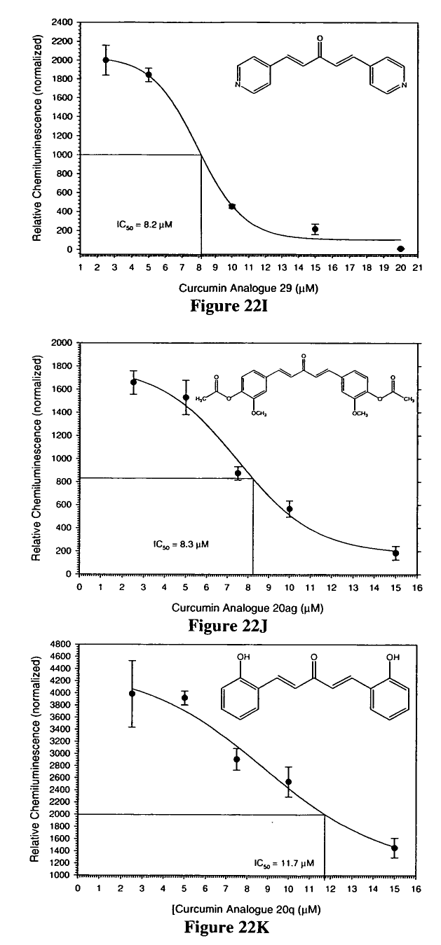

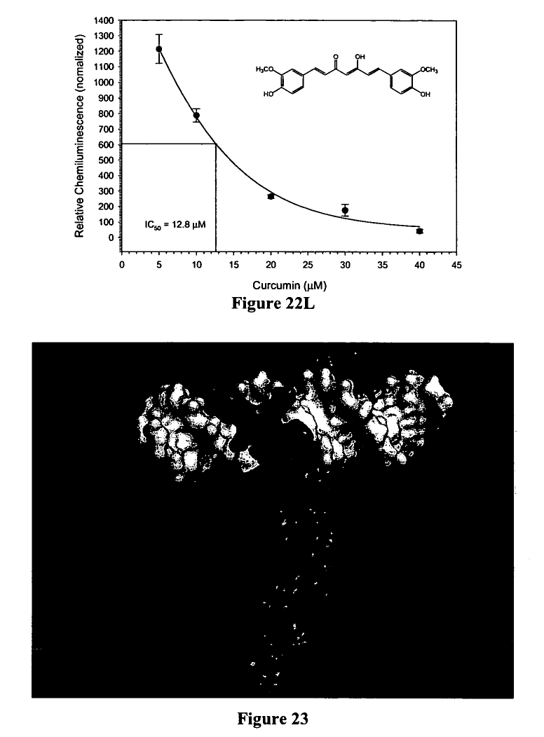

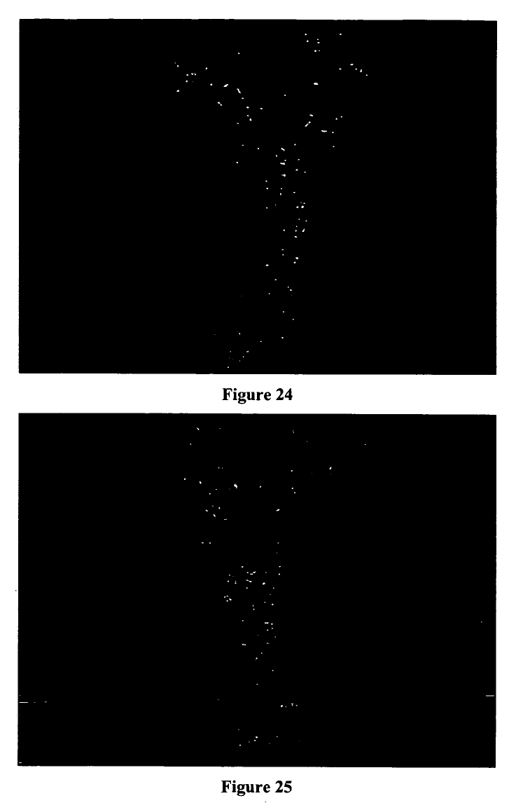

FIG. 23 shows the c-Jun and c-Fos AP-1 heterodimer bound to a segment of DNA. In FIG. 23, the blue protein is the c-Jun/c-Fos heterodimer and the yellow segment is the DNA. When the DNA is removed as shown in FIG. 24, a “Y” shaped area is exposed. It is in this location that the analogs will bind if they are preventing an AP-1-DNA binding interaction. When docking studies were performed, the potential inhibitors bound in the entire DNA interaction region. The front side of these binding interactions is shown in FIG. 25 and the backside of these binding interactions is shown in FIG. 26.

The analogs that appear to be coming over the top in FIG. 26 are the same analogs as in FIG. 25. Most of these analogs bind in the exact region as the DNA was bound. However, the analogs have mediocre Kₑₛₜ values with analog 9b displaying the best inhibition with a Kₑₛₜ of 6.53E-8 M as shown in Table 10. There is no correlation to the Kₑₓₚ results. This indicates that the analogs do not bind to the c-Jun and C-Fos heterodimer or at the very least, they do not inhibit DNA from binding to AP-1.

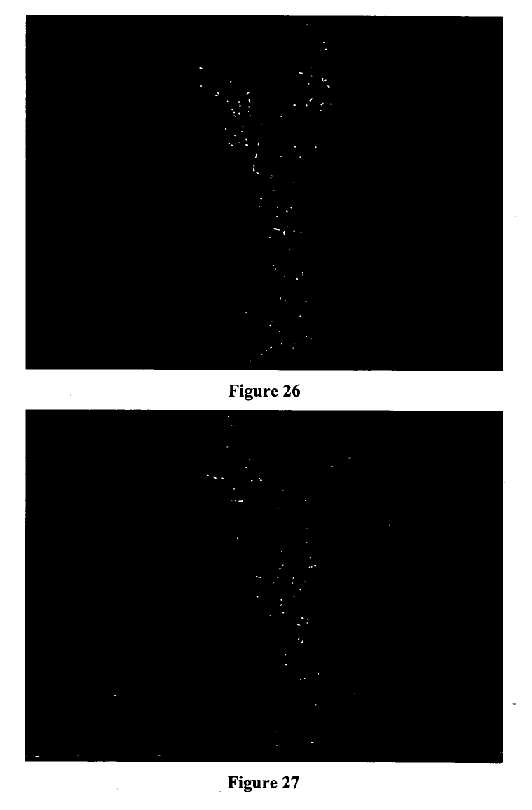

To verify these findings, the MES program was utilized on the AP-1 heterodimer to identify any potential binding areas for the potential inhibitors. The results of this docking are similar to those from when the MES program was not used (FIG. 27). All of the analogs still bind to the area directly below the DNA binding area and indicate a possible inhibition of AP-1-DNA binding interactions. Once again, the potential inhibitors have mediocre Kₑₛₜ values with analog 15a having a Kₑₛₜ value of 1.26E-7 M as shown in Table 11. However, there is no correlation to the Kₑₓₚ results which indicates that the analogs do not bind to the c-Jun and C-Fos heterodimer or at the very least, they do not inhibit DNA from binding to AP-1.

TABLE 10
Kₑₛₜ Values for AP-1 (1FOS).

| | |
|---|---|
| 9b | 6.53E−08 |
| 12b | 9.96E−08 |
| 15a | 1.57E−07 |
| 3d | 1.71E−07 |
| 6a | 1.73E−07 |
| 53 | 1.95E−07 |
| 46ad | 2.37E−07 |
| 20m | 2.43E−07 |
| 23 | 2.44E−07 |
| 20d | 2.88E−07 |
| 20v | 2.99E−07 |
| 48ad | 4.18E−07 |
| 13c | 4.38E−07 |
| 9a | 4.49E−07 |
| 3a | 5.01E−07 |
| 25 | 5.31E−07 |
| 15b | 5.38E−07 |
| 3i | 5.42E−07 |
| 20i | 5.49E−07 |
| 3g | 6.19E−07 |
| 14a | 6.61E−07 |
| 20o | 7.31E−07 |
| 38a | 7.53E−07 |
| 3h | 7.73E−07 |
| 20z | 8.46E−07 |
| 20w | 8.98E−07 |
| 20k | 9.23E−07 |
| 20ac | 9.70E−07 |
| 20g | 1.07E−06 |
| 6b | 1.16E−06 |
| 20ag | 1.20E−06 |
| 20l | 1.28E−06 |
| 17b | 1.28E−06 |
| 3e | 1.43E−06 |
| 20ae | 1.53E−06 |
| 20y | 1.60E−06 |
| 20q | 1.60E−06 |
| 13a | 1.75E−06 |
| 36e | 1.83E−06 |
| 52ac | 1.85E−06 |
| 14b | 1.86E−06 |
| 3b | 1.97E−06 |
| 3f | 1.99E−06 |
| 20u | 2.07E−06 |
| 20ah | 2.13E−06 |
| 52l | 2.14E−06 |
| 20t | 2.35E−06 |
| 11b | 2.45E−06 |
| 36a | 2.54E−06 |
| 20c | 2.62E−06 |
| 20aa | 2.72E−06 |
| 20ab | 2.76E−06 |
| 16b | 2.82E−06 |
| 52e | 2.92E−06 |
| 20b | 2.93E−06 |
| 52aa | 2.96E−06 |
| 29 | 3.12E−06 |
| 40af | 3.24E−06 |
| 20s | 3.30E−06 |
| 31 | 3.38E−06 |
| 20n | 3.57E−06 |
| 20a | 3.93E−06 |
| 38b | 4.02E−06 |
| 46al | 4.17E−06 |
| 20r | 4.30E−06 |
| 13b | 4.60E−06 |
| 20p | 4.90E−06 |
| 45a | 5.01E−06 |
| 20x | 5.14E−06 |
| 46ak | 5.25E−06 |
| 20e | 5.33E−06 |
| 39b | 5.88E−06 |
| 46a | 6.66E−06 |
| 52b | 6.83E−06 |
| 42b | 6.84E−06 |
| 20f | 7.63E−06 |
| 50b | 8.05E−06 |
| 48a | 1.10E−05 |
| 34 | 1.11E−05 |
| 45b | 1.19E−05 |
| 43b | 1.91E−05 |
| 40b | 1.99E−05 |
| 35e | 5.48E−05 |
| 35a | 5.53E−05 |
| 35q | 1.02E−04 |

TABLE 11
Kₑₛₜ Values for AP-1 (1FOS) with MES.

| | |
|---|---|
| 15a | 1.26E−07 |
| 13c | 2.05E−07 |
| 23 | 2.96E−07 |
| 46ad | 3.15E−07 |
| 53 | 3.43E−07 |
| 3g | 4.75E−07 |
| 20d | 5.74E−07 |
| 9b | 6.11E−07 |
| 25 | 6.78E−07 |
| 20z | 7.14E−07 |
| 20v | 9.80E−07 |
| 38a | 1.04E−06 |
| 48ad | 1.08E−06 |
| 9a | 1.12E−06 |
| 20w | 1.20E−06 |
| 20ab | 1.22E−06 |
| 15b | 1.22E−06 |
| 20ag | 1.24E−06 |
| 20o | 1.26E−06 |
| 3e | 1.29E−06 |
| 20u | 1.29E−06 |
| 16b | 1.30E−06 |
| 20g | 1.38E−06 |
| 17b | 1.49E−06 |
| 3i | 1.53E−06 |
| 3d | 1.57E−06 |
| 3h | 1.59E−06 |
| 13b | 1.74E−06 |
| 12b | 1.79E−06 |
| 6a | 1.83E−06 |
| 20s | 1.86E−06 |
| 52aa | 1.98E−06 |
| 6b | 2.02E−06 |
| 3a | 2.03E−06 |
| 14a | 2.04E−06 |
| 20ae | 2.07E−06 |
| 52ac | 2.13E−06 |
| 20m | 2.17E−06 |
| 13a | 2.34E−06 |
| 42b | 2.58E−06 |
| 20n | 2.60E−06 |
| 46al | 2.74E−06 |
| 20c | 2.76E−06 |
| 52e | 2.79E−06 |
| 20y | 2.93E−06 |
| 3f | 2.99E−06 |
| 20k | 3.28E−06 |
| 20aa | 3.29E−06 |
| 20x | 3.33E−06 |
| 20ah | 3.40E−06 |
| 11b | 3.58E−06 |
| 20ac | 3.67E−06 |
| 46a | 3.76E−06 |
| 14b | 3.78E−06 |
| 36e | 3.79E−06 |
| 31 | 3.81E−06 |
| 20i | 3.86E−06 |
| 52l | 3.95E−06 |
| 3b | 4.00E−06 |
| 29 | 4.08E−06 |
| 20a | 4.26E−06 |
| 20l | 4.65E−06 |
| 40af | 4.80E−06 |
| 39b | 4.97E−06 |
| 20e | 5.33E−06 |
| 20q | 5.40E−06 |
| 52b | 6.00E−06 |
| 46ak | 6.04E−06 |
| 20b | 6.33E−06 |
| 20p | 6.38E−06 |
| 38b | 6.95E−06 |
| 20t | 8.29E−06 |
| 34 | 8.51E−06 |
| 20f | 8.58E−06 |
| 40b | 8.61E−06 |
| 36a | 9.23E−06 |
| 20r | 9.23E−06 |
| 48a | 1.04E−05 |
| 50b | 1.07E−05 |
| 43b | 1.09E−05 |
| 45b | 1.45E−05 |
| 45a | 1.78E−05 |
| 35a | 4.51E−05 |
| 35e | 4.90E−05 |
| 35q | 1.10E−04 |

### Example 7

### Evaluation of Curcumin Derivative Pharmacophores using QSAR

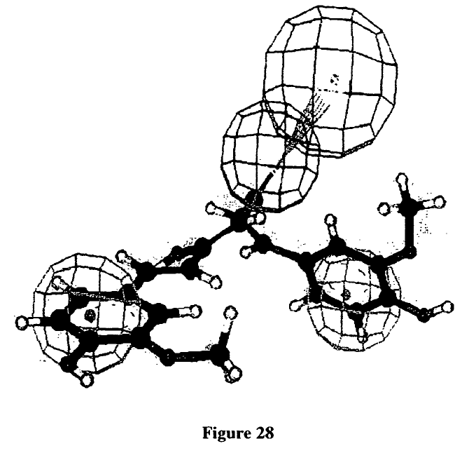

A QSAR analysis of the data was carried out using the Catalyst program (Accelrys). A wide range of structures and activities from the results described for FIGS. 3-5 were used to generate multiple pharamcophores. A single pharmacophore did not provide a satisfactory fit of the data. Moreover, pharmacophores that were derived separately from 5-carbon analogs or from 3-carbon analogs did not provide satisfactory fits. However, a single pharmacophore could provide a satisfactory fit of the data for analogs in the 7-carbon series. FIG. 28 shows a pharmacophore on which curcumin is superimposed. In FIG. 28, Curcumin was aligned with the pharmacophore model generated with the Catalyst program, using compounds 3a, 3e, 6a, 9a, 12b, 14a, and 14b as the training set. The pharmacophore model consists of two hydrophobic aromatic regions with centers 11.8 angstroms (Å) apart and a hydrogen bond acceptor 6.2 Å from the nearest hydrophogic aromatic region and 7 Å from the other. The pharmacophore provided an excellent fit (correlation 0.9) of analogs on the 7-carbon series. The inability of a single pharmacophore to provide a satisfactory fit of all of the data or of the data from the 3-carbon or 5-carbon series may mean that there are several different targets for these analogs.

Summary of Curcumin Derivatives Activity

Curcumin has a broad range of biological activities, some of which may derive from its anti-oxidant activity or ability to quench free radical reactions and some that involve inhibition or inactivation of specific targets. Curcumin can scavenge superoxide radicals, hydrogen peroxide and nitric oxide, and it has been suggested that the ability of curcumin to protect against radiation damage, iron-induced hepatic damage, xanthine oxidase injury and oxidative stress depends upon the anti-oxidant and free radical-scavenging properties of curcumin (Joe et al., Crit. Rev. Food Sci. Nutr. 2004, 44, 97; Bonte et al., Planta Med. 1997, 63, 265; Reddy et al., Toxicology 1996, 107, 39; Cohly et al., Free Radical Biol. Med. 1998, 24, 49.) In Example 2, the abilities of curcumin and derivatives to quench the pre-formed radical monocation of 2,2′-azinobis-(3-ethylbenzothiazoline-6-sulfonic acid), known as the Total Radical-trapping Anti-oxidant Parameter (TRAP) assay, and the abilities of these compounds to reduce the ferric tripyridyltriazine complex, known as the Ferric Reducing/Anti-oxidant Power (FRAP) assay, were demonstrated (Schlesier et al., Free Radical Res. 2002, 36, 177.) It is noteworthy that many of the most active derivatives with regard to NF-κB show no activity in the TRAP or FRAP assay, which leads to the conclusion that there is no correlation between anti-oxidant activity and ability to inhibit the TNFα-induced activation of NF-κB. While not intending to be bound by theory, the lack of correlation between the anti-oxidant activities of curcumin and derivatives and the abilities of these compounds to inhibit the TNFα-induced activation of NF-κB and the PMA-induced activation of AP-1 suggests that curcumin and its derivatives inhibit a specific target (or targets) rather than function through general redox chemistry.

In summary, derivatives of curcumin in which the two aryl rings are separated by 7-carbon, 5-carbon or 3-carbon spacers are able to inhibit the TNFα-induced activation of AP-1 or NF-κB. However, activities can vary widely. The most active derivatives retain the enone functionality, although this functionality is not essential for activity. In addition, derivatives with the 5-carbon spacer are generally the most active. Ring substituents are not necessary but can affect activity. In addition, the aryl rings can be nitrogen heterocycles. The inhibition of TNFα-induced activation of NF-κB by curcumin and derivatives may occur at the level of the IKK complex.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

What is claimed is:

1. A method of a treating a subject afflicted with cancer or a precancerous condition comprising administering to the subject a therapeutically effective amount of a composition including a compound of Formula I:
Ar¹-L-Ar² (I)
wherein:
L is a divalent linking group comprising an alkylene or an alkenylene comprising 3, 4, 5, 6, or 7 backbone carbon atoms, wherein one or more of the backbone carbon atoms form part of a carbonyl or secondary alcohol;
and
Ar¹ and Ar² are each independently aryl groups.

2. The method of claim 1, wherein either or both of Ar¹ and Ar² are independently heteroaryl groups.

3. The method of claim 1, wherein the divalent linking group L is unsaturated.

4. The method of claim 1, wherein L is an alkylene or an alkenylene selected from the group consisting of: 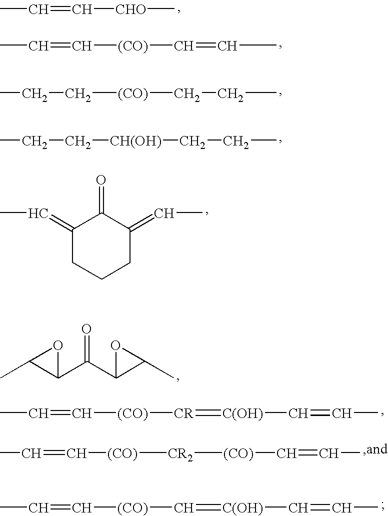
wherein R consists of an alkyl or aryl group comprising 10 carbon atoms or less.

5. The method of claim 1, wherein Ar¹ is a phenyl group according to Formula II: 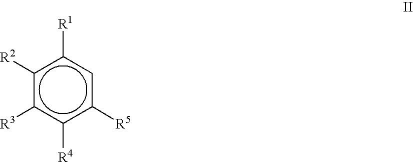
and Ar² is a phenyl group according to Formula III: 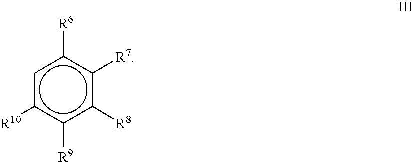
and each of R¹—R¹⁰ is selected from the group consisting of hydrogen, hydroxyl, methyl, methoxyl, dimethylamine, trifluoromethyl, chloro, fluoro, acetoxyl, cyano, and carboxymethyl.

6. The method of claim 1, wherein the composition further comprises a pharmaceutically acceptable carrier.

7. The method of claim 1, wherein the composition inhibits AP-1 or NF-κB activity.

8. The method of claim 1, wherein the cancer comprises tumor cells that constitutively express activated NF-κB.

9. The method of claim 1, wherein the cancer comprises tumor cells that constitutively express activated AP-1.

10. A method for identifying an antitumor curcumin derivative, comprising:
contacting a cell including activatable NF-κB with a curcumin derivative;
contacting the cell with an NF-κB activator; and
determining the effect on NF-κB activation by the curcumin derivative;
wherein a curcumin derivative that reduces NF-κB activation is identified as an antitumor curcumin derivative.

11. The method of claim 10, wherein the NF-κB activator comprises TNF-α or IL-1.

12. The method of claim 10, wherein the cell is a cancer cell.

13. The method of claim 10, wherein the curcumin derivative is a compound of Formula I:
Ar¹-L-Ar² (I)
wherein:
L is a divalent linking group comprising an alkylene or an alkenylene comprising 3, 4, 5, 6, or 7 backbone carbon atoms, wherein one or more of the backbone carbon atoms form part of a carbonyl or secondary alcohol;
and
Ar¹ and Ar² are each independently aryl groups.

14. A method for identifying an antitumor curcumin derivative, comprising:
contacting a cell including activatable AP-1 with a curcumin derivative;
contacting the cell with an AP-1 activator; and
determining the effect on AP-1 activation by the curcumin derivative;
wherein a curcumin derivative that reduces AP-1 activation is identified as an antitumor curcumin derivative.

15. The method of claim 14, wherein the AP-1 activator comprises TNF-α, PMA, or an MAPK kinase.

16. The method of claim 14, wherein the cell is a cancer cell.

17. The method of claim 14, wherein the lowering of AP-1 activation by the curcumin derivative occurs by direct inhibition of AP-1 activity.

18. The method of claim 14, wherein the lowering of AP-1 activation by the curcumin derivative occurs by indirect inhibition of AP-1 activity.

19. The method of claim 14, wherein the curcumin derivative is a compound of Formula I:
Ar¹-L-Ar² (I)
wherein:
L is a divalent linking group comprising an alkylene or an alkenylene comprising 3, 4, 5, 6, or 7 backbone carbon atoms, wherein one or more of the backbone carbon atoms form part of a carbonyl or secondary alcohol;
and
Ar¹ and Ar² are each independently aryl groups.

20. A method of a treating a subject afflicted with cancer or a precancerous condition comprising administering to the subject a therapeutically effective amount of a composition including a compound of Formula IV:
Ar¹-L-R¹¹ (IV)
wherein:
L is a divalent linking group comprising an alkylene or an alkenylene comprising 3, 4, 5, 6, or 7 backbone carbon atoms, wherein one or more of the backbone carbon atoms form part of a carbonyl or secondary alcohol;
and
Ar¹ is an aryl group and R¹¹ is an alkyl group, a heterocyclic group, or a hydrogen.

21. The method of claim 20, wherein one or more of the aryl groups are heteroaryl groups.

22. The method of claim 20, wherein the divalent linking group L is unsaturated.

23. The method of claim 20, wherein R¹¹ is a methyl group.

24. The method of claim 20, wherein L is an alkylene or an alkenylene selected from the group consisting of: 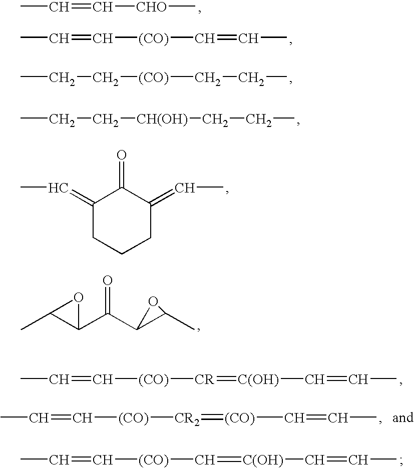
wherein R consists of an alkyl or aryl group comprising 10 carbon atoms or less.

25. The method of claim 20, wherein Ar¹ is a phenyl group according to Formula II: 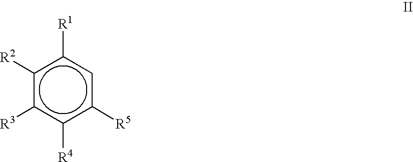
and each of R¹—R⁵ are selected from the group consisting of hydrogen, hydroxyl, methyl, methoxyl, dimethylamine, trifluoromethyl, chloro, fluoro, acetoxyl, cyano, and carboxymethyl.
